SEQUENCE LISTING (11 sequences)

SEQ ID NO: 1 - DNA, 41380 bp, Homo sapiens
cagctgaggctgccagccctggaaattcaggggactgggatacaggccatgggagccacccggggctatgtacaaatgggatggaatttcccacaaagtgcagtggtaactgtctcagcgacttccctggtgtgtcctgaaacgtacaatgtatcctacatttacccaggctcttcctgggagcaactctttgtgcagggactcttctgtgggtagcctctggggggcttttaaatctggcttcttggtttgaacaaaagatggcagagagagagggagagagagagagaactctggatcagagggtgggcacttacttaattccaccaaaaagttcctgattgctctccagaaaggctgcaccagtccccatgcaccccaaggaaagtactctcatttagctatattttcaccaacaaatggtctcacccaacttcctattgttgccagtgctccaggtgtagggctgtgtctcaacctcattttaataggcatttctctgattactagcgtatgagtgtctcttcatgagcttggtagctatttgggctcccccttctgtgacctatctgttccaatcctgtgccagctcgctggctcctgcctggctactctactctcccttccttccctgtctgtgataactagtacgccaggttgggctaggctaggaaccactgagtctcttgggatggttttgttcgtttctgctctgacccattatttgaatgtcacagcttaatgcccaggaggtgagggtactgtccttgtttccaagttctctggtgggggagcacctgaggatttttcccccaggaaacacctacaccttccctgtatccagcagtttccattttccagtaacagagaacacctaatgtcaggcactaggcaaggcccatcccactttctctctataactatgctggagggagggactgttatcctgctggagagacagataagaaacaggtatgtaaaggtcaagtagaatgaccaaggtcatgtaacttgtaagagaccccactaggatgctgtctacagcttaaactgttagtgcatctccagtaatgtctatttcaaataactgctatgtcccaggtggctacttcctaaatagaatttagaaattatcattctcaattattgaaatgtaatgaaaaagtaaactcgggtaccacctttgtacctcctcagtccccagtgtctaaaaaaggtgcaagtgagtggagggagaggagggagagttactttcctctagagaggaagtaactctagaggaagtaatgtttctttgagggttactcaaaggaggaagtgaagtaaaaaggagaagtaacaccttcatgagggtttataagggaggggcctgtctgatgctggacagcatgctttcctccatgaggaaataactagaaaagaagaggaagcggaaagaaaatgtgcaaaggccctggggcagagaaatgctgagaggaaccactgtggccagagggggaaggcgagggctaagatgtttgagaagtggccgtggaggctcttcaggctggaatggcaggacactgagtgtatatgaagatgtcccgagccgtttgagaccctcaattttcatgtcttccattctaaagcttatctggctgatatggacctgggtggggacatgcacatgctcctgcatggcctcccctctccacagtctcccatcttcccctgggcaaagaaaggctcccttcctatctaacctgagtctgacccccttcctgtatacagtgtcctgaggcacaaaaacatcctcggcaccaaacagagacacttttggtgtctctttcagcaagactccaaaaatcccctctctatccatcccttaagtaatgcacttttaagacaattttcctttttatgtttaataattatttatcagaatacatatttgtgtatgatgtcttgatcacttaaataccattaatcattgttataatcacataatcctaaagagaattttagagccttattaattaatcattaattaatttttgaggcagggtcttgctctgttgcccaggctagagtgcagtggtgtgatcatggctcactgcagccttgacttcctgggctcaagtgatcctcccaccccagccttccaagtaactgggaccccacgtgtgtaccacgatgctcaactaaattttaaacttttttgtagagggtctcactatcttgcccaggctggtctcaaactcctggcctcaagagatcctcccacctcagcctaccaaaatgctgggattacaggcatgagtcactgtgcctaaacttttttttatactttcaacttttgttttggattcagggggtacatgtgcagggttgttacactggtatattgagtgatgttgaggtgtggggtatgactgatcctgtccccctggtagtgagcatagtacccaagagttagtttttcaaccttttcccccctccctccctttcccctttagtagtccccaccagggtctgttgttgccatctttatgtccacgtgtagaaaattttaaaaagctaatgcatattcacatatttattgtggttcacataatagataggtgatgaatacagaatttaaacaataaaagcattagggtaggatacaattctgttggaaaacggaatggaaacatacttaaaaggggtaaagaacacaatgtaaatattttgactgttaaagggatacttgtttatattcttaagtataacatggcagttttgtattcttatacaaattcctttgggtatatttaaaagagagagataataattttatacataaaggtcaataaatacaatataccctatttaaatatatgattaaaaaactcattttagatgccaacttgaaaatgtgtaaggcaaacatattgttaaacattttttagggggtttcaacaaaaagtctgttataggccatagtgctctaggggtggtgacagtgatccggaggcgggaacgccgggctggcattgtccctggagccatctacatccagctggctgggctggagtttggtgtggcttccagtgttcactgctatgatgctgctatcaggtcagaatgtcagcagggatttgtctaggctttactgtctccccagcattaaccatagtgcctggcacgtagtaagagctcaacacatattcagcgcatgaatgaggggccggcacttacggagctctcactttgtgccaggcactgtgctaagagctgcctatgcattcccctggaattctcacaaccacgctgcagtaggtccagtgagtatccccattccagagatggcgaaactaagccacaaagaggatacataaccagcccaaggccaccagtcaaggaagtagcagactgggacctgggcatcctggcaggcaccctttaccataatgcctctctgtccccatgtggtccctggggtagagtctgcatccttgggttctgaaactccagagcaagcactctggggcactccctgagtgataccctcctgccatcttaaacggctctctgccatctgcttcctcctcctaagtggttttccttctgtcctccctgtttaatgtttaagttgtgcacatcctgtgacatcctggagacccatgttcctgacctacttacctattcaaccatctcttctcacacatggggtagcaatgagaagcacagggggtctctgcaagggagtagggaggtggagagagcgggggcagccacacatcacactgctgttctcagtggcctcctgctgtcaacaagctaacattaataaaacaagcccactcttctgctaagagactgtgacagggtaacatttattctaggtagcttttcaagaaaacagctcttgctcccacggtcccctcggagggcctgcagagctccagcgatgtcagagggcatctgtttgtttctcctttgcttataaaagggacctggaaatcaaaggcaggctcttagcccttagcagtgaggatgacagctctgcaccagccacacgcaggcccctggctctaggaaccacctctcacccagacgcccaggcacagcatggtggggagagagtacaggacacagggcctaggcctgctgccccgcccacagcccgccccctagccctttctctgaggccaggtgtactcacctagcctccagggtcccctgctcaggtggccccagaggcccaaacccactgctcccactttctgagacctgggaccctcacctccccaactcccaccatagggcgacactgaaaagtgtccccttcccccccccgtttccaagtcaccagcttgaggatgcccaaggctcaaaggctcctccgagtggcaccccgcatcgggctgcagaggtcggagaggggaggggatggtgggtgcaaagggtggcttcttcaggaaagggaagcctgtccctggctcccagcacctgtcacccctggcgatctcaccccgcggccatcattcccgtcctcacggcccatttcagggacactgaggctcaaagagccacgtgatttattcaaagcagctcgagccaccacttggagccacaagaaagccctggtccgtgcctcctcgccttggtccctggcccttcactcttcgtccaggctctctgacctctttccctctgcgcccctgcctcttccccaggcctgggtccccaaggcactcggccacgcgctgggtccggggaagaggcgcggccgcggccgaggcggggacagggaggcggcagcccctcctgcgcagcgacggcgcgggcgggcggaggccggggcgccccagccccccgcccccgctccctccggccgtgacgtcagagccggacggggtaaactgagcggcggcggcggggcgctggggcggagactgcgacccggagccgcccggactgacggagcccactgcggtgcgggcgttggcgcgggcacggaggacccgggcaggcatcgcaagcgaccccgaggtaacacggccccggttccctgctcgcagggcgcggtgccggaggggagcgggcccgggggaggcagggaggcagggaggcccccagagctacgcgggcggggctgcggggaccccggggcagccggggcgcagggcgcagggcgcaaggtgcacggggccgagcgtgcagtttgggaggtggcacagttgggggcggcggggatcagggcagcgtcctgggggttctaggttgggtctccagattgggtccccgacggcgctcgcgggcgctctttccagggagcgcgggctctcgtggggacgcgctcgaggcaggtgagccgaggctggggcgcccgggggtgtccccggatgccggtgcgcgcggctccctgacccgcccttccgcggcctgggggacgggagaatggggggtccacggccgccttgggtcctcaccgcgcaccgcgcccgcagcggagccccggagccatggccctgagcgagctggcgctggtccgctggctgcaggagagccgccgctcgcggaagctcatcctgttcatcgtgttcctggcgctgctgctggacaacatgctgctcactgtcgtgggtacgtgcggacagggcacccctgccccggcaccccagccccagcgccccttccccggcactccacttacccctgcgcggtcccggagctccgccaaggcccgggaaagttggagaactcgctttgcaaaaaagacatcccctccaggctgccgcgccggggctaggaggagccgcgttgtgtggcttcgctttcctgatgctcgggtcaaaaactgtgacatccgataagacccagaacttatgtttccccctgataatttcccgcatcctcttgcctgctttcactgcgatgaaaaagatttctgttccaaaagagaaattcctaaagttcaaaattacgtgttagaataattattgggctactcgaaaaggcaggttagtaatttggtgtagtacctaaaagagcaagtttcgatcccttactccaaaaacaagacatttagatatactgtattagtgaatgacttgagatggaaatatttcaggaaccattgcaagaacaatttgaacaatcttggttctattatttttcctatagtggaattttgaaaaattaaataatcgaaatccaggtcccttaattaggcccattaacagagaagtatttggcatgcatttccttaaaaaaaaaaatcccagtttttaatgaatgaaaaaaaaatcacgactcatttagactgtaagtggtgggagctgtttcctttctctccatttgcaggttttctcacagcagttttcagagcatctgataggtttcatctcgaggagttatcagagccgtggagctcatccctgttcagtaccaacggcattagttctgcttcggctcgaatgctccattggacacatgtgctcacagcttcgattatatgtttcacaatttaatttgtattttttgttgttgctttttattttggtttttgtttgtttttttgaaatggagtctcactctgttgcccaggctggagggcagtggcacagtcttggctcactgcaacctccacctcccgggttcaagtgattctcctgcattagcctcccgagtagctgggttaccggcatgccccaccaaagctggctaatttttgtatttttattagagacaggattttgccatgttggccaggctggtcttgaactcctgacctcaggtgatctacccgcctcgacctcccaaaatgctgggattacaggcctgagccaccgtgcctggcctgtttcagttttgctttttccttgtgaaagccttaggaattcttaaatcttacaagttgtaggaaaaattacagattcccactcataatagaaggatgaagtgggcctgttgcagaatttccaagttattacttcctgcaggttaatttttttctaagacacataatgaagtgtagttttctgtttccttgattcattcatttgaaagtcttttgagcctctctttcttagaagcactggttgactccaggtcagcactctcttaacgtcaggccacattttgcttttctgaaatattccttgaaggaacaatgtggaagaaacccacaaatgacagaatctctgggaccagtcagttcagcacagcagctgccttttgagaggagacagttctcagggccgttgggggcatggttctttctagtgagctaagtaaagtgaacttgctccaatgtcgttatagacatatggcacagggctcctgttttggggtagacctgtcaccctctggacctcttctggcatttgacacaaccatagttttgttgttgttattgttgttttgtttgtttgagacagagtctcactctgtcacccaggctggagtgcagtggtgcaatctttgctcactgcaacttccacctcccaggttcaagcgatcctcctttctcagcctccaaaggagctgggattacaggtgggtgccaccacgcccagctaatttttgtatttttagtagagagagggtttctccatgttggccagcctggtctcgaactcctgacctcaggtgatccacctgcctcagcttcccaaagtcctggaattacaggtgtgagccactgctccctgccgacacaaccatagttttttgctggcttttttcctggagagggtttcagtgtgatcaccaccttgccattctgctcttatccccagtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacaggtagggcagactactttagtcaaagagtttgatatttgtatcagtcctagcttatgtccttcctggaattctgcttcttactcattggtgagagtctggaaaatccatggtgggtgagctggggaaaacccagtccccttcccctatgtctctgttcccagataaccacaatttcctattctattctcccttctgttgctcaacaagtaagaaaatattaaagaagacagaataaagaggacctcttccaccaagtgggaggactttggcctgtctaaatgccaggtttcgtaagtgcatttgtcagaatcgcccttggcagcaggaactctacagccaccatggctgtcatcctattttatgagccatgtggcctcctcttatctcctacctcaggccatgcacttctctgtagagagccaaggtgtcttcttttggaactggcatgttaaagcctctcacagataccaggtagggagtgtgggaaaaggttggacgcactggtgcgcagatggagctccgagttgatgccgaggtcatgggctgtgttcttcccttctgctggctacgtagccacgtccacgtgcatgaaaggaagagaactgaggctgttgtctggataatccaagctgcccaaactagcttcagatgaatcaggctgttctagatttttttgaacccacattgtttggaataactgtttacttctccattgttggtgaagtgtttgttcttctgtagctgaggtggaccagttctgcctggtccgtgggggctcaaagctcaggacaagggccttgcagctgagaggaggaggcaggcccccaaggtgtcagtgcaaggagtgggagctggtacagttaagtgttggtggtgggctgccaagtgcgcagtgtagctacctaggtgaaagttacaaaacaccagaaatggtggggaggggtgtgcccagagtcctacctgcagaccaggcccagaagcaggaacaaagactagactagagatggaagagatggtggcgaggagagggtggaactaaggaggcaggggaaaccagggctgggctaggctgtaagaggccccaagtgctgggctgagaaaggagtgggattctgtgtggggcagttgggagccattagaaattccaaagccaaaaagaaatgagtgaatcaagcactattttaagcagggtaggtgggcagggagcagggagaacttcacatttcctctgcctttggcgtggggagtggggcgcaggactggctgcataatttgtgggtctcggtgcaaaatgaaaatgtgtgccccacattcaaacattatgaaacacttcaaggtggcagcagagcattaaatcaaccaagctctgagagcagagccctgtgcgactctagggttaggggcaccgagaggtgaggaagcctggggtggtggctggggctcaaggaggggatggcagatctggccttccgccagcacaagctgccttggacacatggagcttgagtcggacaggagcacattctcaacatctgcagttgaattttctggatggaagggtgccattgaattgatttcaacaatctcaagccatgtccagccctctagtctgtgcagagcaatattctaaggctgctacataaaacatgctaagcaaattaacccagcgaagggctgactgtgagcccattaagaaggtgacattcagtgactcctctgttctcaggagaaaaaccaaaccgacctcagtagttgcaggattgttttctttccttttttcaagactaagaatgcttcttaaaatgcatctggtataattacagaaaatagctcagggttttgtattctgagaaagcaaacagctcaaatattgtatgtgaaaattgaggacattatcgtaataataccttgccaatcacttgggtggaacgttactagccgtacagtgtaatacttagaaattcggctgaaatgatgtgagtactccaatcttccaaacgcaaatggcaaatgaaaagcttttttaaaaatcccattcttatttttaatgctttgcttatgccatggggaagaggcttgtttaaaaccagtgagctctttcccctgttaaatactccaacggctttgaggctgtagccacaccaggctcatgtttgggacttcaaaaacattatcttccattttgtgacttgtgaaatttagtctattctcatggtgagtaatccactttagaaattatctagagcaaaattgtaatccccaactgttttgtccagctaccgagcaaatttgaaaatgattttctagtgctgcctggtaatatggaaggacaaacaaatttttttttctttttttttgagatagagtcttgctctgtcacccaggctggagtgcagcagtgtgatcttggctcactgcaacctccacctcctgggttcaagcaattcacatgtctcagtctcctcagtagctgggattacaggcgtgcaccactatgcctggctattttttgtattttttggtagagacgaggtttcaccatgttggccaagctggtgtcgaactcctgacctcaggtgatctgcccacctcggcctcccaaagtgctgggattacaggcgtgagccaccgccctcagcgagcaaataaattttcatattgtgatcctgatttgtaatttgtctcataatttccaagcccagtagatatgaatttggggactgggcagctcttctggacactccaagattctgcttcccaccgcagttcagatggattcagagaagggacaaatgtgcattccatttgcctttaattgtcttcaaggagttaacttgtgagccattgcaaacagtcttttgatgtaaactgtgccaatcacatcctccatgagcaagatgtatgactcaagtcttcagtgttgtaaacaaatgtgtttaaaggctttttaaaattggttaatatgtttctgattaaagagtagttaaaaatatggagcatcagagcttgcagagacttttcctgaaagcttttgggccagtcctagcattttataggcaaaggagtttgcagttcaggaggagaaagcatttaccacaacagtggcaggactgtagccacatgttccgactgtcccatttcaagctttgttcttccccgcttcctgttcttcccacctgcttacaattatacagcttgtacctttagctactagaaataaagaatgcagctcaaaatctgtctttcttgcactgacttgggctcttcctggttgtaccaggcagaactgctggagacccagtgagttgctgattttgctacttttaatcaatgagcagagagtttagaaaagatcagcctgattcctgtggacatggaacctccctggtccctgaaatattcagtctttttccagtccctgatgtgaactggtccagcctcgtgcatccctaaagatgtgtggggaaagcttcctgtagtcaggctgggcagagctattcttttccaaagctgcttgttttaactgaagtgggacccactctgaatcgagacattctacaccacaccacaccataactggaatcctctattcacagtcgtgatggtgccatgtgtgtggcttttgtgggtaggagtcacaaaatgtaggccgcctgtttatttatttatttatttagacagggtcttgctctattgcccaggctggagtgcagtggggctatctcggctcactgcaacctctacctcccaggttcaagcaattctcctgcctcagcctcctgagtagctgagactacatgccaccatgcctggctaattttttttgtattttagtagagatggggtttcagcatttggccaggctgatctcgaactcctgacctcaagtgatcctcctgcctcagcctctcaaagtgctgggattacaggcgtgagccaccatgcccggccctgtctgttttttttaaatattcgataagttctcacaccaaaccctgaaaatatagagtaacaattcttgggttcatttaactgctaataggatgcacattcttcctgtttcatttgttattcagatcctgtatttctgctgaaaatatatgaccagttaggtggcaaaaacctttgatttgggtgtgtgtagtgatgacctggccaatagcacactttccaagaaagcctgaggttgctggattgacctgatttccatgttggaacctcttgcctcctattttataattttggctgaactattcatttcatgattgcagttgtgtgcaggtggtaccagaaattatactagtgatacttggtcatcatttggtctaagaagcaaattttagatatatatgtccataagaggaaagttccaggcacatgaacaatgggacaacacgtttacatttctctcttccctcagtcaggtaacgatatggcattgtggtcttgcttagcataaggttgaaataatagttgcatcttcagaaccaaaagatattccacaccaaacgactggcttgggaaagagttaacttcattgctggggaccgtttcccttgcttccttcttagcttgtcattgtctcagtgatctgctttttatcaagccagcgtcaatggcttaatttatttccatagttacttagtatatttgaaagcagttctttttgcttgcaaatcttagctaacatctgaacgacaaggaggaagccacagggcatgaaatctctacgaaatctaccatggttttattttatttttttgctagcttcagccagtgggtctgaagtgggctgtgtaggaacaagcagtgaccttttcagcttctctatggacagggaaaccggctgttctgagaagagatctcccatcctgcatgtggttaggatggtagggagggcttgatgagtctttgcttctgaaatcagaggaaactggctgtggaattagaacattcctaggagaagcaaaagctctgttaagctatgtgtcagagaacagggctcacgcccaggaaagtgagaagtccgtgccccctggaattctctggtagctgaaaaagacaagcagaacctaagaattttgacaagcggaatcgaagaattttatttactaaatgattgacaaacctcctactatatccaggcaaattgaaaatatcattccatttactgacattcatttactgcacaccttttaggagtgtgtagggatcactggaagggagctgttgtctctcgttgcatctggtgatagtttgcacccttcggggctctgtagtctgtatgcttcttgtaatttaccacggtagaggaaactgtggttttagcaatcatgagaagagagcttccattagtctctatggcaaggataagctaattattaacactagagaagtattgtcttcattgaccatttagggacagatggggtttctccaatgactaagtatatataattaacataacttttattgttttacatgtttttctagggattcattatgtgatttttagtcttactcaaattggattaactgcttcgtgatgctctgtagaggatgaccgtgggccagtatgacatttggctaaaggtttgctgaatcagatctggctctgcttagcaatactttaattgagaaggccctgaattgagacaaggggggcaactcagacacagattgtgactaatgtcagttgagtttctgtctgtttaggagatactgtggggaagcagaaagggtttggtccttctgcccaccactcaatgactagggacgctggacgactctccttctatggtgggtgcccgaggggccagaagagtttgagaacggagacccaggagcttcccagggagtcttctttgccttgaagccgagctctggggaaaaatgatgcccagcgcttaccactgagcagcccatgcagtggcttggcctgtgttgaaagtgcaggcacaggtgtgtccggtactgaggaggcggcaacatgagcctggttgtatctgaagccattggcttgctctgcagccatgggtgagccactctgtctctgaacctcagctttctcatctaaggtgagggtttggactggctagcccttgagtttccttctagctgtgatattcgtttattccatacgtgcattgttaaaatcccaaagaaaattaaaaagaaaaacagtagctgcagtgtttctaaagaaaacacagtacctcaaatggtaaaaagaaccctctgtaggcacaaacgctgtggttttcagtgaaactgtgcactctccatcccatgtgctccagcgtccccacccaggtgcacatcagcagggcctagcacatgttaacacactcagggtatgcccgtagccttgaatttggggcaagtaattcacctccccgagcctcagtgttgatcctctgaaaaatggataaaaccagctaccctcccttggaatctcaggccacccagcatgctggttccctgcctacagtcgtgggaatgtctgagtcttcaggtagggagggacctcagtggcttccagcccaccacagctgtgccctgatgggttacctggaatactggctggactgaacagtgggcacagtatggctggtggtttgggggccacagacaagggttgccacattttattgagttttgtcctgaaaccacccagtgtttctgcctacagtctgggaaagggccacagatgtcttatttgccttcagggatgtccctgcagcctgaagggttccatattagagccttataaatagccaaaatgaagaactggtgaaacaagagtgaagcccctaattccaatagctttgatacgtattggtacttcaacaactagattttccaaatatatattgaacagagggagaagcctgtgtttattccaggacagcccggaagattgtcatatgcccgttctttcatgggacaaactgtagttgctgaaaataaattgcctgtgagttctcctctctgagcctcctggacgtttcctgattcagtatcctgcagtgccgtgtgagcccccggtgttgggtttcttttgtccacatgaataaggggtacagagaatcagctctgggtgggctgagacagaagaagctgtcacccgatggccatctgaactgcaggtcagaatcctggcatcttctctggtttacccaatttgcttggggccccactttgtgaattacaagctcatctctgttcacagcaggggaatctttgcatggtttgtgggaggggactgactgtctgggaatctgtcatgcttgtgagttactctctgtatttttggtatttggtttctggatacaggtgaagcagttaaaaaatatttgaatatggtcaagagaacttttggaaagattctatttaaagctatatatgccgtttgggtactggcggaggtggtaaaatttacaaggaaaagccatgtgggaatggcccaggcgaaattagtctgatgacatctgataagtaatgacagatatcatgcctcagtttcctcatctgtaacatagggaggtgaaatagggcccttattaaatcatgactaaatctggtgggtgtaaatgcttggagaactgagtgtaaactttaaatgaaggcgctatgattattttttatttatttatttacttttgagacagaatctcgctctgtcacccaggctggagtgcagtggcatgatctcagcgcactacatcctctgccttctaggtttaagcaattctcgtgcctcagcctcccaagtagttgggattacaggtgtgcactactatgcctgactcattttttgtatttttttttttttttttttttttttttttagtacagacagggttttgccatgttgcccaggctggtcttgaactcctgagctcaggtgatccgcccacctcggcctcccaaagtgccaggattacaggcatgagccatcatgctcagccgattacaattttttattaaagcctggaatggaagaacccttccaggctgttgccagagctcaggatgcttggcccttctgccttcctggattcatttcattcctctccaggttcctgagggtccagagctagagacagaagagacttagagatgtttcaggctcttctgggttggagcaaaccttcaattgggcaagagctcagccactgttggcttcctctcaaggcagaggggctgggaagctgcctgtaccctcatgccccacccacagagcagggacccagctgtctacttaatccagcagaaacagaggacaaagaagaaagagaatgagggaggagatgacatttcttaggcaccttctatgctctaggcactggtgttaacagtttaacatccatatctctttctgtactccagtcctgggaggtgggcggtggtctctccgagggactgagaggagacctccctagattaagtggcagggtcatgtgtccagccagacatggcataggggaatgccagggtctttccaccagccctgttgacctggcgccagagtcctaggtgagcagtggtctcacggagcaatttggattttgaaattttgcacagaaaatgagactttgtctttctgggttggcctcatggggtcaacaggaaggcagatcttaacatcttgcttcctaatgcctcttagacatactgcagtctgccagtggaatcacttgatggccttttacccgaatcatttgattgcagtggctcctttgggagagctttagcaaagactataattaaaggaatcattttttaaaaatgacagaaataggggccgtgtattttggatgtgttctgggttacttgccaatattgtgttaacatttattccaaacaaatttctgttcacagtggcagtccataaatgtattcacgttccaagacagtcaaacacaaaaaggtcacataggaagcactgagtatatgaattttcagaaaagtccaccaaacacttatctgtctgatcccaaagggtagccaggcgaaaatcaatataaagccttaaaaaaatagcctgcagtcaccagatttgtctgatgtttgtgttttgcagaattggctatccaattcccatatttgcgggattctgcatcatgtttgtctcaacaattagtaagtgtgtggtgttttccttctgagtgtgggtctcatcagggtgggcattgatgcccatgagccgggaattaacaatacaacctaaggacggcgggggggcggggggggtcgtggttggctcatgcctgtaatccgagcactttgggaggctgaggtgggcggatcatctgaggtcaggagtccgagaccaacctggccaacatggcaaaaccccgtctctactaaaaatacaaaaggtagccaggtgtggtggcatgcacctatagtcccagctactggggaggctgaggcaggagaatcacttgaacctggaagacggaggttgcagtaagccgagattgcaccactgcactgcagcctgggcgacagagcaagactccatctcaaaaaaaagtatatggcctgggaggtttgtgtattggcaacaattgcagaaaggaattgatagctttttacggaacaaaagcacagggtggctaacatgcaaatgctccactgggttaaggggggcttctgaaacgttcctgtttgggacggttgtgtcccagcagcactgatgtgcggtcttggaccccctctctcggcagtgtttgccttctccagcagctatgccttcctgctgattgccaggtcgctgcagggcatcggctcgtcctgctcctctgtggctggtaggtgtggaatgcctgagtgagttcgtgaggggccccttgcagagtgagctggactcattcagggaattcaggtattggtggtggttggggtgctggggattcttggagaaggcacccactttcctcttgggttctgcttggtcttacattttagtgaatctgacaggtttgggaagatcacaaggctggctggagagggaaggcgagtcacgcattattcttttgctgttccccggagctggcctgttgactatttctttccctgtgtagggatgggcatgcttgccagtgtctacacagatgatgaagagagaggcaacgtcatgggaatcgccttgggaggcctggccatgggggtcttaggtgggtaaggcccccgtgtaggcaaactggcaagaggggcctgcctggtgctggacagcggcagtcctcgcccagtgaccctggcgcagtttgcactgacacagaggtcccctttttatttttttcctttcctgctttcatggagactttgaaaaatggtaggaagagccctgagctgggggtggaggggatcaggacttcccctgcctctgccccaaacctccctgggcccagatggaaaaccaaaggttgaacaggatggtctgccagaccctttcctcgggaacattcagtgagttgtgatcacggagaaccttgctttctgggtctgcctcccccagctgccctggctgacttcccagaccaggggacaaacatcttccctgagagggattcaaaaccttttccttcctcttccgatttcccgcctgcttccaggagcctgcagccttgcctgcgctggtgtacaaggagcgaagtctctgtttacacattttcctgtgcagtggaagcagcccagccctgggcctggatcttgtttctctcgggcctttgctttcagggtgacctttatagactcctgcccatttcttaggaaaaggcacttggaggaagttgccaagacaactgaactctaaccgaacagaacaatagggcagccaccccaaagccttattggaacaaagtagagagagaaacacaagagtcaaatagatggttctagtacagggagagggcatgtgtcccaggggtggtgtccccactttctctccctgcagtgggcccccccttcgggagtgtgctctatgagtttgtggggaagacggctccgttcctggtgctggccgccctggtactcttggatggaggtgagtgagtccacgtgggcgccatgccatgaccttggcatcgtgctggcacgcgcttgggccacacctgctttctgaagaggggcttgtcttttttatttttattttttagctattcagctctttgtgctccagccgtcccgggtgcagccagaggtaagcggctgggaatgagggccctgggggagggggcatggtgctgcttctgacccgtgtttttttcttgacagagtcagaaggggacacccctaaccacgctgctgaaggacccgtacatcctcattgctgcaggtggggctctgtgggtcttctgagtcaggggaatgcgaggtgatggccgcgtgctggaggcaggggtggatggccagggctgattttcgttttgctgctagaaatgttgggctatataaaaaaacagaaaaggcaaagaggctacaagtcaaaggaaatggttttcttccgccattgctttctgacactcagccaaggccctttttgtctcatttggggaaaatcaaaatggcgtatgctgggctctgcagagctgcctgggtcctggggatgcccggctgggtttgtttggagctaagcttgcccactgtgtttgtatggaaggccatcattgtttggccttgaactttccggaaggagtccttggcctcaaggcaagggtccccggtgggcagtctgactcaccttggcatatatgaatagaaactgacagcagagccccacagggaccagaggggctcccttcttgcagagagcatccctgccgagcagcagggaggccgttcaaacaccctttggctaaaggacatgctttggcctagaggggctaaaagaaagcactggggttttgtgactcttgggaaggataatggtctgcatctatccaagggactgttccccaagactgtcccagtggtaggaggctgcctgctttgccaggccccgggctgcctaaccagggaccagccttgtgtaggtgttgctgggcctcagtaccaaggtaagcagacccttggggtcaggccaggagaaggtctggctgcgcgctgatgtgttggtctctggggcagcctgcactgtagcactagacccagttagggttagacttgtctaagcacggctccctaacacaggtaggctggagggcgcctgggagaactgggcaccagtagttaagttcagcctttgtcaaccctcaggctggttttgtttaacccttctgctgctggatttgattagggcagcaggtatgtaaccctggccaagaccctgtctatcatgcttgctggccaaagggatatggaggtggcatggtgacttattcatgcagtgacgtctgcaagatgcagggggcatcccagagctcagatgcttctggaaccctggcatctgaggtggggacgaggaggtatggtgagggacgggggctccagaaatgtgacttcctgaacctcgggcacgtctgagcagggctgtgcagcctgggcctgtcctgtcttgggcttccctggaacctcactgcctagtacctgctcacaggagacacaaagatgctacccagcactcctggactgtcatcgacacttccagacagcccgactggggaagggagttgagagccaagtggctgtccacttgaggggagtcctgccattgatatgctctgcctttaagtgtcatgtcgaatgaacagacctttcttcaccatatgtgcaccttcatcacgattgcacaatattctttgaatttgttgtagcaaattagaggttccaagaggcacaacacgggaagttcccagaacgatggagaacagttccgtgggtctttttccagatagttctatatttacaaagggttctggttaagagatcatgaatatttgcataggtgcccttatgtaccgctgaaccccgccccctccacctaacggcatgcctgtactgaatgcacctgatcctgcttggattttctggcatttctaggtatcattagaaatgcccacgtatattgcacagtaaaacacagatgcgagagcctcaggctgccccgagactgtgtgtggtctggaagtggcctgattgctgggtggccccaagcaagctactgcattaataaactgggcttcctttcttcattgataaaaccataattgggtaggttaaaccaaaggaactctagggctgcctccagtccaaactttgtctctgggattctagttgtttaaaatgaggacgacgacgaccatgatgatgatgattatgataaacagcctttgctgtccccttgatatgcgatttatcctttacttaatgaaagtagaagttgcggttatctgagctgtaggcgcatggagtctaactgtgcctggaaatgagagaggaggcagaagccactacaaagccctttcctcccttacaggctccatctgctttgcaaacatgggcatcgccatgctggagccagccctgcccatctggatgatggagaccatgtgttcccgaaagtggcagctgggtaaggactggggtgggctcttctgattcaagagcatttgtccccagggcagcctttgtccccagggcatccctgctgagttgcccttatggggatgataaagtgattgcttctgtttgactcttaatggggtcattgcttagggcagccttggggtctgtttgttctctggtttatccatcatccctgaggggtgcagaactcacacagacacatcagatctgggcagaaatgagagtaaaaatacagtcagcatcttcccctgtttctgaactggactaactggacagccataaaatttatgtgaaagagactaataacaaactaattgttaaaaggattaattcttaaaaagtatttttaattttgaaaagtaatacatgcttattataacaaaatcaaagtgtgctgattgaggacgtaaaacatttttaaatttcatgctcatctttctccctcccccagcccatctcctgtgatattcattgttaacagttctatgtctttttctagatggttctgtgtttaccaccctattgcatgaaatgcctaaaaatccagtcaagtgaattttgccatttacatcactgtgtgacctcttgtttttccttcatgcaattttgtggccatgtttccgtgtcagaacaagtagatttactttttaaaactaattaaattttatttttttttatcaaagttacacatgtacataattgtaagtcaaagataaaagaattataaggaaaaacagctgtctttttcccacgcctcttgatccccaaagcccttcagattccatcaccttcaacgtttttagatctttcttctgctgatacccttatgtctctaaataatatgtgtacacccccgactcttttttttttttttttttgagacaaagtctcactctgtcacccaggctggagtgcggtggcacgatctccactcactgcaacctctgcctcccgggttcaagcgattctcctgcctcagcctcccgagtagctgggattacaggcacgtgccaccacacctggctaatgacaccccctttttaaaaattttccaatttctgacattgtccaccagcttcctaatacagtagatggggattatttaatgccgttgcagcgccacatagaagtatcaaacaaccagctcacacattttcccatcttctgtcctccctgaataattgcattacagcttcttgttagactggcattcagcactgacattattgtgactgggtaattattgttcacagctgactgagctatgtagggtactgtgattaatttcctttctttttgcaaatttaattttcctggagttagtcgttgccttgtcctttcattttcttaatgttttatgtagtgtttatgaatatatccaccaaagtttctggcaaagatgaaaaattctccaattgcagtcaagtgttggcaaacctgatctttctgtgtatttgctcgtttgtggatgtctgtcctaggtcttcagtctagacttccttccccatcaccccagcatcttctgtgcctctcttctgtttcctgtattccttgcccttgactgacttcctccttattgcagagcatagcctcccacagcatagcctcccacagcttcctgagagtgtgcgtggcaggtaaaggtctctgagaccctgcatagttgcaaatattttacccttatcctacactttgttgactttgtctgcatgtataattcttggttggaaatcatttcccttgggagttttcgggccactgttctgttgtcttctagcatccagggttgctgtgggggaggccagtgttgccctcattcctgagcctctgtaggagcccgagggacctttacttcttctctaggttataaaagtccatgttgatgatttgcagtgggctctgttttcctcctctgggctgaacacttgcttaatcagccctttcacttggggaaccccatgttacaattctggggagactctttgtattattacttgaatgaatccccctattttctctcctgttttactccagttggctgagcgttaggctccctacttttattcttctaatttcttgtctttccccttttccattgagttattcctgttcatcttttaaactgcttattgagtttttaaaattgatgctatcatatcttaaatctgaagagccttttgtgtataccctgagtgtttggggttttggtgaggttgttgttattgcaaaaacaccctcccaccatgttcctgtttcagcctcttgtgttgccccggttgccattctggttaatttctgtggagaaactgtctcctgccagggtgaaggctggagtgctaggggccccactgctcttaacacagcctctctgcctgctttcatgttgacagcctctgctttcagccccaccttcacagtacccgaagcttcagttcctttctggctgtgaggcacaatggctcttgcctcttgctggcctcaccatttgcaggtggaagattccagcattctctgcggtgtcctctgctcacttcttcctcgtattgtcccttctcctctcccactctgtttgtcctcaggaaggtatgggggcggaagggggcataggctgtgtgtctagaattttcttctattcctttgctagcatttaaatggactttgaggtgggagtggagaaaaactatagattcagtctgccctgtttaactggaaaccttttattctttttaatggttgcatattgttttattgttaaaataaaacaacatataactgttgaaatcctcagtcagtccctattattgggcatttgggctcattttgttgccagaaaagggatctcaatccagaccccaagataaagattttggaaatcactcaggaaggaattcatggcatgtcacagaacacagtagaagaggcaagtttattagaaacaactctgttagagcagggcgtcctcagaaagcacatctggactttctgctgttgtaggagagtgtccttgtacgtatctttaggctgtttccttaactctaaacatctcatgactatgggtcgtgactggcaaggaatgtgctttgctagttttaaggtggagctgagcttaaaatggtgtaacttctgcttccttaacagttccagtttttgccattagaaaccatatggcaggctgggccggtggcttatgcctgtaatcccagcactttgggaggccgaggcaggtggatttcttgagaccaggagtttgagaccaggagttcaagaccagcgagatcctgtctctacaaaaacaaaacaaaacaaaacaaaacaaaaaacccaaaaacagcaacaaaaaaattagccaggagtagtagcgcacacctgaagtcccagctactcgggagattgaggtgggagtattacctgagcccaggagacagagttttgctcttgttgcccaggctggagtgcaatggcgcgatcttggctgaccgcaatctctgcctcctgggttcaagcaattctccctttttctttaagaatttattgacagtccttcctagagatttatattctttttatagttctttcaaggtacgtgtggatagcttcagatggagactctcagctctgaatcaagcagacattttcaatgtccccaacccttcttcaggtgaccttattctctcacaggcatccagccctggggaatgggggtgtcttgccagaagctgccccctggcttagactttccctgttctgcctgcctccctgcagttgggctggcctgagtaccagtgtctttggaatctgcagagttgctatgctctctcatcgaacaatacctaaaaagtcccaaatggccgggggagagggggtaaggatggagagaaactgctaatggggtgtgaggtttctttgtgggatggtgacaatgttctgtaatcagatggtgatgatggttgctcaactctgtgaatacactaaacatcactgagcgatgcactgtcaaagggtgaattttgtttttttttgagacggagtctcagtctgttgcccaggctggagtgcagtggcacgatctcagctcactgcaacctctgccactcgggttcaagcaattctcctgcctcagcctcctgagtagctgggattacaggtgcctaccactgtacccggctaattttgtatttttagtagagacagggtttcactatcttggccaggctagtcttgaactcctgacttcgtgatccccctgcctcagcctcccgaagtgctgggattacaggcgtgagccaccgcatcctgcccaaagggtgaattttaaaccacgtgaattgtgtcataaaacaaaacccaaaccaagcctgacatcagaagaggtattcagtgaaggaggaaggattcttggaattaaacaagaatgtttcttaggcagaaaatttagctctgaactccctagcagccaagatgaaagtggaagcccaaagaccttcagagctcctaaagaggcgtgccgcttggcaggaggggtggacttggatctgacaccgagtcagcaaacagcagatgagaagacaccctccagccttgttgctcatacccgctgctgacactggagaggctgtttcccagcttttcttccctctggcccttgaggtgatgtggatctctcggtgttttcaggaggcgcccgctgtccaagtggggtgtgcactgctgagtccagctgctgtgttgactttgcatgcaactcttgagctgagaagctcttatttgtttatttatttttagagacagggtctcgtcctgttgcccaggctggagtgctctggtgtaatcatggctcactgtagcctcaacctcctgggctcaagctgtcctcctgcctcagcctctcaaagtattgggattacaggcgtgaactgggtctcccggccacctagctgaactctcatcctctttgaatgtgtcctggccatgtgcaggacacttaagatgttttgtgctcatcctcttcagacaaagcctgaggctttctttataaaatctttaaagtcagccagaagccatgggccatcaggggcagatagcagagtgcaggctgcctggatctcatcagattgctgtgtggggcctatcaccggcgccagacgttcactcaacccgcttgctgcacatttccaccaccgaggttcctcctttcatttgccccagagatagacatgtctgtctttaagagaatcaggtttctctacctgcctcctgtgtcagcaccagcaggaggggaaataccccaccttccctagttctctgtagctttcagcccatggctccctgaccctggggatggggtactgtgagtgaagcagtgcggaggacccagtgctgagggcggggagctgcccctgcagcccagactctggtgttcaggaatcttctctgagctcgctccagtccagggtgaaagggaggctgttctgcctttatgcttcccttcactgtttctggagaaagactatgatatttgaagtcagggcaaacaatcttattagctgcatgggtgacacaggattgctcttgctgaaggcattgtttttggccacattttttaggggtagtaaagatggcagcctacccatcttccacgtcgctcccttgccagcctgccccccaaattcctcatctgccacttgaggggaagccctgggcttgagtgggtggagaaagaatgatttcatggtgttttctctatatctgagttctaggtcagatggcctctccagaccccttgagacttagggacttacagtttggggtgaggcctacaagcccccaaagggaaactgaagccctggacaacaggccccagacaaggatggaggcacactcttgtcttctggttcttctagcccctttgtatttaatgagctgtggggatgcaaagtgggatggagggtaggacacgggcctgtaccagaagacaagagggactgccagccttctgtggctctggggtgcgaggacttaggatcctcctaagcctgccataggacagggcagaccattgcccctattctggttctaggccgagcccttggccttcagggaggggtcatcttgagcttggaggaaacccaaggtctcatttccacctttgcttccggggctggatggccagctcatctccccgcaggcttcgtgatgaccggagcccctctctcactggctggccaaggccgtggctgaagccagattgtaccaaaatgtagaagatctccctttggtgaactttctggtgtgagcagagatatgtgaagagtgttgccccgcagaagggagggttcctgccatggcccagggcactgttgcaggccagtcccagccaacacttttctcagagtcgggagaagccctagaaagaattcttgctgaggctgggtgcgtggctcatgcctgtaatcctagcactttgggaggctgaggcaggtggatcacttgagcccaagaggcagaggttgcactaagccgagatcgtgccattgcactccagcctgggcaacagagccactgacattctgcctctattgagactccacctcaaaaaacaaacaggatcttccaatactggaactgcagtccctcaggggagtgaatggccacagagagtggcgtctgccttctagatggagtctgggccctctgggtcaccacagatctctctttgctccatttttcattttatttctctgacactgaggctgaatgtcagtggttctttatcatggtatctgcactgttatttctgcttgcaatttgcttgttttattcatttatacctacctggcctttggggattcttatcatgagacaaatgttgtcatgatatcttactatccggactaacagtccctagagctggtaggaagtcctacgacacagcttttgaggccaggtttatataccatctgctctgtggagcttggccagacgcagttccaggtccccagagcatgacacctgtgtttttgttatgttccttacttggccaggtcacagtttaaacgtcatctgctcctttccttcaaaacacttgcgtcaggacaagaatcacgttgtcattgtctttgctgcaggccactgctgtgctgggcacgtggctgtcagtaaatgtttgctcaatcagattacactgaacaattatttgaccaccacatattgctaagcatgacagaaataattctctggaaaatgaaatgagattgctctttattagtgtttcccagaactcccatgtctagaatgatctggttttggatttagctcatggagccatttatttggaagtccctgctgtgttggaagactcagcctgtttctgggtttgatggtagacgttcgtgctttatcctaaagagacaagatcacacttgcccgtgagtgtgtggccagctgctgggatgagaaggacctccaggccccatgcattttgagtgtagcaggcactcggtcatctgatatctacctgcatgtctcacttatgcagtcctctgattgagaacaaagtaacttttactatgtgtgggcaaggcttggaatcaagtctggtatatctgatttcaaggaaaaaatactaaaatcactaggagtttgaaaagcagtcactcataaagagattgtcctgtggttctgaggaccacacacagcttggatggatgaggaccagacacagctgatgtggacaattcccctgtagccaagctgggtctgataatggttaggcctttctgttcaatgcggaggacaggttcaaggccactgctgccaccaccagcagggaatcaccctgggcacatcagaacctgggggatgcaggtgtcctctatggggacagcaataaaacttggataggaggccggacgtggtggctcacgcctgtaatcccagcactttgggaggctgaggcaggcggatcacctgaggtcaggagtttgagcccagcctggccaacatgttgaaactcagtctctactaaaaatacaaaaattaactaggcgtggtggcgcatgcctgtcgtctcagctacttgggaggctgaggcaagagaatcacttgaacctgggaggcagaggttgcagtgagccaagatcgtgccgctgcactccagcctgggtgacagagcgagactccatctcaaaaaaaaaaaaaaaaaaaaaatctgacttgacaacaaaaaatgaccaggcagacatttatgtttctgacatagaaaaatgttcataaagctaactgaaacaagcaggatagagtataatcccttttttttgtaaatcaaaacacacataaatatatttatatatgtaaataaacttacaggagaaaataactgtggtgattatctgagtaatggatggggtttggggtaattttaacatttttctttttgtttatctcacggttccctaattctttcaatgactgcgtcttacttctgtcggatgaaatcacacataagattgcaggtgctgagctcccaggcaggagctgtttgtattggcccacgtgtgaagcccaccaccagccagagatggaggcaagatgaagcagttgatagagaaaggactttcatttctggagtgtctcagcagctttgtgtggaggataagccacgtgggtccagaccggatccggcaggcgccggatattagctgctggggtgtgggccccggggcttcgttttatctgctctcatcaacattgtggtgtctacagttattactttttctaacatatgactgatatcagcactgataaggtctccttttcataaatttacaggcgttgccttcttgccagctagtatctcttatctcattggaaccaatatttttgggatacttgcacacaaaatggggaggtaagatgatatgaaaacaacactcattctgttacaataatgtagttctttcaaaaaattctaagttgtttctgcctttgctgttacaagtaattgttttgattttcattgaaaatttggactagaaaaaaatcaaatgatcatttcttgatggtgctttttcttttggtaggtggctttgtgctcttctgggaatgataattgttggagtcagcattttatgtgtgagtaaaagatggcatttgacaagtgggaacaatctgtgaataaaacttgcgctttgggcaagaatcaccaagaaacattttacgtttgagtttgttgatgttttattacagctttcattttattctaattttttatcatcttaaaaatgtttggacaagactttccaaggagtgtattttcattttatgtcagaaattatttaaaatgttttatttcacagtttaacttttcccctgcatcatcttaagcttgacagtgtcttaaacacttcaggacaaccaacctgaaacaatagcaagatgtttttctagaataaaatctcttctaccaatggccaatatccatccctcatgaggcctacttgttcttgtcaaaacttgagggtggactaggctgatctttcacatatgccggtgacaagtttttcatggtggtcataaaactcgtttcaagctttgacttttaaagatttggagggtccggttcttcacaaaccccacccttcttcctcctgttttcttcagaagggaacaggcataccacgctcagagaaaacacaagttcaagactttccgagatgatttagagttggatctcttgttattttagcataatgtttatttcctcttacagattccatttgcaaaaaacatttatggactcatagctccgaactttggagttggttttgcaattggtaagtcacacgaaccttgtgcctacatttaaaaccgcttttccactaggaagggttcttcttcctctggtagactgtagttcctcaacctaaatttccagaatcttagaaggaggctgcaggcagcttagctaaattacagcctgtcgattctccgtgtaaagacacctgcttctatgcactagtcatttatttcctgaaaaccagtattttttaaaagctgatatttacagaaggaaactgatgctccactgctggggctgggcaaacaaaggaatcccttgcacagagctcatttttgtggactcctccctatgagcaggtcttctcatgaggctgggggcaaaggcttcacatatgtattttgaagaggttttagtacagacaaagcagactaatgtgttgatgaccgcctgtgctgcaggaattcccatgggcaaggagatgggcttttgtaatgttaagaatgataatatgttaggaaaaatctgtcactattttgagtgaatggtatacttttgtctcttatttataatataccaattgatcatgataaaaataagtgctcagtccacccctgtagaagcaggaacgcaacctttgagtggtacagtggcacagcatggctttcactgaggacagatggtagaaggctgctgaggctggggtagaactcagtatggtactctaggtccttctcaggtgaccgtttgcctgtcctctgccttgcctccccttgtcctccactgtctggttggcccatctttttagggagggtgctagtcctctggcggctccccttgccattctgcaggtctggcagagttgcaggttgctaactgtacggcccctggcctgcatccttttggtgcttccctgctttgctgttttctggttcacttgtccctcaccactcagcacacctgcatgcctgacccacctcctgccagtcacacctgggccacacgcttagactatgggtgtttccaaggtggagaataataggggggaccagaacgccccagaactccaaggagtccgagagatcatctggccctgctcagaaattatgtgtgtgtatgtatgtttatgtgtgtgtgtgtgtgtatgcatgtgtatccatgtatgtgtgtgttgtgtgtatgagtgtgtatgtgtgtgttgtgtgtatgtgtgtgaactaaaagaagctaaaacacacatgcacacacatacatacacatacacactcatacacaaacacacacattcatacacacaaacacacatacacacactgacacacgcatacacacacacatacacacatacaccccccacataaacacatacacatacacaaatatacatacacacatatgcatacatacatacacgtagatacacatacacatacaaacacccacccacatacatatgcacacatacacacatacatatacacacatacacacacacctacacacatacacatacacacacatacacaaatacaagtacatacacaaacacatatacacagacacatacacatgcaaacacatgtacacacatatatacacatacatacacagatacatatatacatatacataatacacatacacacatgcatacacacatatacatagacatacacagatacatatatacatatacatacatactcataaatacacacatatacatacagacatacaccccagcaacaaccacgaccagcattagaatctcgtctcttgaatagtggggagcaggtagcataaagatggtgtgtgttcagagttcctggctcagactccctggctcagtgagtgtggtcaagcctcgcctctctgagtattggtttcctcatctataaaatggggatgctcatgattggctgctgtgagccagtgtctgtgaacacactggcagggactcagaggttggtgccctcccctccacatgtgtcagtggcaggcacatgaggaaagtttctttcattgaaatggtgcttgctgcttcctaatatgtacaagtgctggggatatgggtgaacaaaaccaacagaagagttgggcatttagcaaagaattgaagaatgaatgacaattgagatgatgctgtgagagaacatctgtatacgaggggaacctgacctcctctgggggtcagaaaaggcttccttcattctttctactcaaagattagtatcaccccaagacttgcaggtggtgatgacagaaggggaagagctggcagggtggtgagtttaagacacactccctgatattcggcccattttgggtttacatccgttttctatacactgtgttccctgactgtcttcaggaatggtggattcgtcaatgatgcctatcatgggctacctcgtagacctgcggcacgtgtccgtctatgggagtgtgtacgccattgcggatgtggcattttgtatggggtatgctataggtaaggacattggcttttcataagaaccttttacctcaatacgtaatggataacgtctactaaaattgtttaaatctttgcatctttcagtctacaagacatttggaagagctttatctaattctctgtttcctgaaaggtccttctgctggtggtgctattgcaaaggcaattggatttccatggctcatgacaattattgggataattgatattctttttgcccctctctgcttttttcttcgaagtccacctgccaaagaagaaaaaatggtaagaaaaatttaggatgcagtgagcatttcttgataattttagcatggccttcctgatagcttcctcatggtttcattctaaagccttcaaacataaatggtgagaattcctttttagtttgtaccacatagagcagttacttcttttatttttttattactgtgaaagttatttctttcaagcttatgtttataaactgggagaatgaaattatttttgtacatagattagcatcagtgttgctcttttgtcgaccaagtataaacctggaatcattcaatttgattttctatggttcttgaaaatcttggacacaatctttctgcctcctgtacttatgcccaaggttagccatttgtggtgctgttgcttttctcataacccttacagcattcagtaatagttgctgtcttgtctttactgtatgtaggattttagagatgattttacgatttttatggaaaaatcattgtatatggactttggggtatatgtgaacacacagaacgatttaattcataactttatgaccaaaattatagccataggcaagatacagagtataaagcatccctcttttgaaaaagtccaaattaattgattcaaaatttgaattttgaaatgcatgagtcattttaacctttctaaggtatttaccttacccagcaaggagacgctcagtgtttctgatcatctttcttatctggtgttgccagtttttaaaacagcaaatgctgataggtacttgtccaggtgaaaggagagtgtcttggattaaggatgaatgaggtgaacaagaggacatgcatttttgtgtttgaatagatgtgaggtttgtttgtaatgttctcttggaagtacatcagaaggtctttctgacagtggaactgaatgcatcttaatgctttggcatttcctgaactgccctttgatgcttttccccaaaaggagatttaaagacaaaccttaaaataaggttatgtgcagcctgtgtttgtcttgggtgttttttacattcattaattcattccacatattttaaaatatatcaagcactgggcactggccttgtgccctttattttaaataggatttaactagatacttttgagtaatgcatggcccgtctgactcatggaaaaggtatggaaggtgctgtggaaataactcagacagaagccatccccttgtccttccctccgcttgcttagatgcttccccagctctgaaaaccacatcaccttctcagacctctcttaggagacatctgctttccagcttgggtgtgtaaatttcagatgcatctttgcttggctgaccctagcataataccttgccgaaatggagtcagaaaattctcagcacaaatgcaaactgccattctgttgccatgataacagtttaaggctgaatggaggttactcaatgtttttgaccaaaaaaagtatatatgtgtgtatatgtgtttgtgtgtgtctgtgtgtgtgtgtataaaatcacctacaaatgctcctgtagcccaggaaactgtttgaagactcctgggagatagttagggctagcatgggcctgggagccacactggccccaccatgtagaggcttatgagtcttggtttcctcctctgttgcatggagaaatgataacgtctacctcataggattgttgtgaggattaaatgaaagaatcccaacagagtgactggccctgtggctcaaaaagtgtgttcctatttccaatatcaccccactctttcctgcttatctaactctctggcttcccccgtcagtccagcaatacctgaattttctccctttttcctcctctgtgatgtgagagtttccttttgctatacagaccccagaactcctgaatgggctccccttgccaagccggcaaacatcagtggctgttgtgatgtccgtttgtgaacggcagcctgatggcttatctgtggccttggggaaatgaatgaggtcagaggatgagcagagaatgtgcttgcccagccaactaccactgtgttccgcagaaggactggcagaaggcaggcctcagtacccctcctctttccccatttaaacgaatacttcagttcatgttttaaaaaaatgctgaaggaagtggaagagacgatcctgaaaggccaaacagtaagaattataggtttccaggaagccactttcactgcaagatgttacaatccttaataagcacctagcaagataaatcattaaaaacttgagaaggtgtgcatctcgctgcccatcaatgatgcccataattgaaggcatcacattggatgccatttgactggcctaatggtgacatttgagagtggtggccagcagttgctgcctactcattgtgcaagcagagcaggaagccagcggtggcggccgacagcacctaatgataagggggcatgcaggcaggacagcagtgtgtgagggaggaggaagaatcaatgaccatgcagttgagaaaccccagagcagacaagtgctggcgttcaggatgagcagttgccaaatgctgggggatgtgagggccagaaggcatacgacccttagcaaatcattcttccattagacagcatcctgataacaaggtgtttggacttaagctgttttatattgtaagatataatgctgttttcatggatcacaagtgacagaatcatgaactagtttataagcatgtgagtttgtcttccatttggtttactggatttctgttttttataatcagcattatcagtataggtggaggaaaaaaactgttactcaagtccctctgcacatacaggaaatatcaacattaccatttgaggaaaagtgtcttataagacagttacacggtagcgcttcactctcaaaattcccagttgagaagagctgtgttgcggatttttttttccaggttgaagaattatttttaaagacaagtcacgcttcttaattttactcttgaagtttctaattgttgccaagacaagaaattaggtggtttgaggattgcagctttcttttcctggatggctctgatagttgttccataaagcccttccagcaatagctctgttttctggatgccatgcgtggggcaaggatggggtggcccgaggctgctgagatagaccacattctgcatattctaagttgtttatagaatttgtccctttgcaggtatttaaatagtgggtgcaaaaccatcattgcttttctagttatgttaaagagaataaaactttcctactcagaaggcccaggacctatcatcaaaatctcaaagactggcactcttatcttttaatgcttttatgggttcactaccccagctttgtattacttatacttttcatttggtccaccatgttggtttgtttgtaccacttaggagatcctaactcatgaaaggggcatttggaggggctggggctcacctctcagtaagagggctttgctctgaattcaaatgccaatcctctcctattgattttctctttggttgagagtcaaacacaccttcctcaccacagcagtgtgtggagagcagatttaatttacctgcctgcactgcggccgccctacttgttccccgggcacctgactgacctgcttgatgctgttcatgcctttcttcatggcacttttgaaacattacatgccacctggcttatttgtgtataacttttttttaaaaagtacaattattgcacaatagatctttcaagagaggtttggaagccagtgagctaggttttaaaggctacagaacatttaaaggaggaaaacttcacctgaaacaggctgctaataaatatgtttttggtgaattgacaaatgctggtcagttaagtatgcatgatgccaaatagtctgactgaactgcttttcctgcagtgcagttttttgttgcgttcctatcatgtgcccagtcctaggttctggggtccttaagatgacatattcctacattcaggtctaatgtggatgagccaagagatggaaggcaattaggaataaggtagtgatgaccgttctttataaggtgacctgcctgcctgggagccgtgcaaggatcgaggccccatgccctgcattccagttgggtgtcccaactggtatctcactgtgcctcctcagcagacctctgtggtggccaggctggcttacttagtgtcctgcttggtaaataactcatgttttactcattgccacctcccttgctgtccaaaccttttccatctttcaaagtttacctcaactcccgcctctataggatagcgttccctgggcactgaaccagcagtgagcactccacctgggaccctggagtccctgtccttgcctgtgcagggaggtaactaagatgctgggataggaccttaaaggcactgagatgcaacacctagtaggtgtcccgtcagctgttgtcagccagatgacagtgctgtagtcttagataatgccatgtaactagggcagtgtttctcaaagttgacactgttgtcacatgaggacagggccctcaaatttcattttttaacctgctccatctctccacccctacatttccttgcgaatcattaatctgggggagatgagacatttattgttaggggggttaaacctttgtgttacatggggagaacggtcctggtgtacaaagtctagtgccgccattattttaaaaattgagctctttattgaatacaagtatcaattcacttggcaagcagcataaaaaacagttaagttagaagctgacaggttgagctggcagcctaacttagagggtcaaggaagtccttatcttcacatgttacacagatcactggataatgtcttgggaaatgataaatgggttaagcactgagtcgagtcttatctggggacaagagctgggagtaatagctaacagacaagatgggttacaataggccaacatcagatacattttaatctatttagatttgcaaactgcaaaaaaaatattggggagtagttcacatggagaaaaactcaagacatacctgataattcattttagttgcacatgacgcccccttgccatcctctgtgctcatctaactaccacggagtgctgcaggcggctcctgtctgccccatcctagctgtacacccctcggtcaaattctttaactgctccgtgcctgttttctcacctcccacagagggttattgtgatgtattgccactaaaatgcttagcatggtcttggcatgataaatgccagctatcaaatataagcaaatactattggacaatctggtttgaaagcagagcagcacgtttttgtcacagtttttaaattttcagtaacaataaaggagggcatgagagtttcatgctcatcagaagaggatttttaaaaaatcatatattttagctagaggacagccaggattggggacaggcggtcaggaaatcacatgtgtgaaaaaataatagctctagcatgttttgcctactaagaaatctaaggggccatgacaatatggtcttaaaagattgggggccaagcatggtcacttgcacctgtaatcctaccactttgggaggttgaggcaggaagatcacttgagcccaggagtttgagcaacctgggcaatgtagtgagatgtcatctctacaaaaaaaaaaaaattaaaaattagccagtcttgatggcatatgcatgtagtcacagctactcgggagtctgaggtgggaagatggcttgagcctgggaagcagcagtgagctgagatcgcaccactgcactatggcatgagtgacattctgtctcaaaacaacaacaaaagattgtaagggcagcctgtgaagatatcagaactggctgggtgcagtggctcgtgtctatagtcacagcactttgggaggctgaggaggggggatcacttgaagtcaggagtttgagaccagcctggccaacatggtgaaaccccatctctactagaaatacaaaaaaagtagctgcgagtggtggtgcacacctgtaatatcagctacttgggaggctgaggtgggagggttgcttgaacccaggaggtagaggttgcagtgagtcaagatcacaacaccgcactccaacctgggtgacgaagcaagacttcatctcaaaaaaaaaaaaaaaaaaaagaaagaaagaaagaaagaaaaagataccagaaccagattcatgaatgcaaacaatggaaacgggttgtagtccagacttaggtacatttctcacatgtgagggcttctctcattaaggagagagcacatcctttcaaggtgtgttgctacaatgacgtctgtaaacagaagttggactgggtgacctccaggatccccataccccaattccaagattctaagtataacaaacctaacctgcctgtcccttgcttcatagagtataagttcccagagtggacttatggcccctgacaggtataatgaaaatactgcagagaccacttctgtctccaattgcctggaatttttagagtgtagaactacaggtcttttaggtcaggcgtctggcgattgctccaaatgactggttaatagcaagtaatactacatagttttcaaactaataatccttaaaaacagtaggttaaaaccatcttatctttatgaaacaagaagttaatatacttgcactttgctctcttttaggctattctcatggatcacaactgccctattaaaacaaaaatgtacactcagaataatatccagtcatatccgataggtgaagatgaagaatctgaaagtgactgagatgagatcctcaaaaatcatcaaagtgtttaattgtataaaacagtgtttccagtgacacaactcatccagaactgtcttagtcataccatccatccctggtgaaagagtaaaaccaaaggttattatttcctttccatggttatggtcgattgccaacagccttataaagaaaaagaagcttttctaggggtttgtataaatagtgttgaaactttattttatgtatttaattttattaaatatcatacaatatattttgatgaaataggtattgtgtaaatctataaatatttgaatccaaaccaaatataattt

SEQ ID NO: 2 - DNA, 1894 bp, Homo sapiens
cgcaagcgaccccgagcggagccccggagccatggccctgagcgagctggcgctggtccgctggctgcaggagagccgccgctcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctccgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactgagatgagatcctcaaaaatcatcaaagtgtttaattgtataaaacagtgtttccagtgacacaactcatccagaactgtcttagtcataccatccatccctggtgaaagagtaaaaccaaaggttattatttcctttccatggttatggtcgattgccaacagccttataaagaaaaagaagcttttctaggggtttgtataaatagtgttgaaactttattttatgtatttaattttattaaatatcatacaatatattttgatgaaataggtattgtgtaaatctataaatatttgaatccaaaccaaatataattt

SEQ ID NO: 3 - protein, 514 aa, Homo sapiens
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 4 - DNA, 413 bp, Homo sapiens
ccccaggcctgggtccccaaggcactcggccacgcgctgggtccggggaagaggcgcggccgcggccgaggcggggacagggaggcggcagcccctcctgcgcagcgacggcgcgggcgggcggaggccggggcgccccagccccccgcccccgctccctccggccgtgacgtcagagccggacggggtaaactgagcggcggcggcggggcgctggggcggagactgcgacccggagccgcccggactgacggagcccactgcggtgcgggcgttggcgcgggcacggaggacccgggcaggcatcgcaagcgaccccgaggtaacacggccccggttccctgctcgcagggcgcggtgccggaggggagcgggcccgggggaggcagggaggcagggaggcccccagagct

SEQ ID NO: 5 - DNA, 2427 bp, Homo sapiens
gcagactgggacctgggcatcctggcaggcaccctttaccataatgcctctctgtccccatgtggtccctggggtagagtctgcatccttgggttctgaaactccagagcaagcactctggggcactccctgagtgataccctcctgccatcttaaacggctctctgccatctgcttcctcctcctaagtggttttccttctgtcctccctgtttaatgtttaagttgtgcacatcctgtgacatcctggagacccatgttcctgacctacttacctattcaaccatctcttctcacacatggggtagcaatgagaagcacagggggtctctgcaagggagtagggaggtggagagagcgggggcagccacacatcacactgctgttctcagtggcctcctgctgtcaacaagctaacattaataaaacaagcccactcttctgctaagagactgtgacagggtaacatttattctaggtagcttttcaagaaaacagctcttgctcccacggtcccctcggagggcctgcagagctccagcgatgtcagagggcatctgtttgtttctcctttgcttataaaagggacctggaaatcaaaggcaggctcttagcccttagcagtgaggatgacagctctgcaccagccacacgcaggcccctggctctaggaaccacctctcacccagacgcccaggcacagcatggtggggagagagtacaggacacagggcctaggcctgctgccccgcccacagcccgccccctagccctttctctgaggccaggtgtactcacctagcctccagggtcccctgctcaggtggccccagaggcccaaacccactgctcccactttctgagacctgggaccctcacctccccaactcccaccatagggcgacactgaaaagtgtccccttcccccccccgtttccaagtcaccagcttgaggatgcccaaggctcaaaggctcctccgagtggcaccccgcatcgggctgcagaggtcggagaggggaggggatggtgggtgcaaagggtggcttcttcaggaaagggaagcctgtccctggctcccagcacctgtcacccctggcgatctcaccccgcggccatcattcccgtcctcacggcccatttcagggacactgaggctcaaagagccacgtgatttattcaaagcagctcgagccaccacttggagccacaagaaagccctggtccgtgcctcctcgccttggtccctggcccttcactcttcgtccaggctctctgacctctttccctctgcgcccctgcctcttccccaggcctgggtccccaaggcactcggccacgcgctgggtccggggaagaggcgcggccgcggccgaggcggggacagggaggcggcagcccctcctgcgcagcgacggcgcgggcgggcggaggccggggcgccccagccccccgcccccgctccctccggccgtgacgtcagagccggacggggtaaactgagcggcggcggcggggcgctggggcggagactgcgacccggagccgcccggactgacggagcccactgcggtgcgggcgttggcgcgggcacggaggacccgggcaggcatcgcaagcgaccccgaggtaacacggccccggttccctgctcgcagggcgcggtgccggaggggagcgggcccgggggaggcagggaggcagggaggcccccagagctacgcgggcggggctgcggggaccccggggcagccggggcgcagggcgcagggcgcaaggtgcacggggccgagcgtgcagtttgggaggtggcacagttgggggcggcggggatcagggcagcgtcctgggggttctaggttgggtctccagattgggtccccgacggcgctcgcgggcgctctttccagggagcgcgggctctcgtggggacgcgctcgaggcaggtgagccgaggctggggcgcccgggggtgtccccggatgccggtgcgcgcggctccctgacccgcccttccgcggcctgggggacgggagaatggggggtccacggccgccttgggtcctcaccgcgcaccgcgcccgcagcggagccccggagccatggccctgagcgagctggcgctggtccgctggctgcaggagagccgccgctcgcggaagctcatcctgttcatcgtgttcctggcgctgctgctggacaacatgctgctcactgtcgtgggtacgtgcggacagggcacccctgccccggcaccccagccccagcgccccttccccggcactccacttacccctgcgcggtcccggagctccgccaaggcccgggaaagttggagaactcgctttgcaaaaaagacatcccctccaggctgccgcgccggggctaggaggagccgcgttgtgtggcttcgctttcctgatgctcg

SEQ ID NO: 6 - DNA, 19 bp, Homo sapiens
gatttgggtcgcggttctt

SEQ ID NO: 7 - DNA, 21 bp, Homo sapiens
tgccttgacattctcgatggt

SEQ ID NO: 8 - DNA, 26 bp, Homo sapiens
ttttaggtttgggtttttaaggtatt

SEQ ID NO: 9 - DNA, 28 bp, Homo sapiens
aactctaaaaacctccctacctccctac

SEQ ID NO: 10 - DNA, 50 bp, Homo sapiens
cggcccatttcagggacactgaggctcaaagagccacgtgatttattcaa

SEQ ID NO: 11 - DNA, 50 bp, Homo sapiens
agcgtcctgggggttctaggttgggtctccagattgggtccccgacggcg

(19) United States
(12) Patent Application Publication
(10) Pub. No.: US 2010/0303795 A1
(43) Pub. Date: Dec. 2, 2010

(54) MARKER OF PROSTATE CANCER

(76) Inventors: KARINA DALSGAARD SØRENSEN, ARHUS C (DK); TORBEN FALCK ØRNTOFT, SILKEBORG (DK); LARS DYRSKJØT ANDERSEN, ODDER (DK)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/788,698

(22) Filed: May 27, 2010

Related U.S. Application Data
(60) Provisional application No. 61/181,326, filed on May 27, 2009.

(51) Int. Cl. A61K 38/46; C12Q 1/68; C12N 5/02; C07K 16/18; C07H 21/04; A61P 35/00

(52) U.S. Cl. 424/94.6; 435/6; 435/375; 530/389.1; 536/24.33; 536/24.31

(57) ABSTRACT

An SLC18A2 gene serves as a marker of prostate cancer. Methods are provided for diagnosing prostate cancer, predicting or prognosticating the disease outcome, predicting recurrence following surgery, and monitoring disease progression in an individual having prostate cancer. The methods relate to determining the methylation state of an SLC18A2 gene and/or determining the level of transcription or translation of the gene in a sample from the individual. Methods of treating prostate cancer are also provided. The invention also pertains to compositions and kits for use in the methods.

### FIELD OF INVENTION

The present invention relates to a SLC18A2 gene as a marker of prostate cancer. The present invention thus relates to methods for diagnosing prostate cancer, predicting or prognosticating the disease outcome, for example recurrence following surgery, as well as methods for monitoring disease progression in an individual having contracted prostate cancer. The present invention also pertains to a kit for use in the methods, in addition to methods of treatment.

### BACKGROUND OF INVENTION

Prostate adenocarcinoma is a major cause of cancer morbidity and mortality in the Western world. The gold standard algorithm for diagnosis currently entails digital rectal exam and measurement of serum prostate-specific antigen (PSA) and if either is suspicious it is followed by histopathologal inspection of needle biopsies obtained trans-rectal prostatic recovering <1% of the prostate volume. However, serum PSA can be elevated in benign conditions and needle biopsy may fail to identify even significant amounts of cancer due to sampling error. The use of PSA testing for prostate cancer detection has increased the incidence of diagnosis as well as shifted detection to earlier and theoretically curable stages. The PSA method, however, is associated with significant false negative rates and does not distinguish well between clinically indolent or aggressive tumors (1).

Indicators currently used for outcome prediction following intended curative radical prostatectomy of primary prostate cancer are PSA, Gleason score, pathological stage and surgical margin status (2), but additional markers are needed to improve stratification of low, medium and high risk patients. Therefore, the introduction of additional diagnostic tests is needed to improve the sensitivity of prostate cancer diagnosis.

Prostate cancer development and progression is characterized by the accumulation of genetic and epigenic alterations. Epigenetic changes seem to generally occur at earlier stages of carcinogenesis and may be more common and consistent (3). Accordingly, mapping of epigenetic alterations could be particularly valuable for biomarker discovery. Several types of epigenetic changes have been reported for prostate cancer including DNA hypomethylation, loss of imprinting, and altered histone modification patterns. The best described epigenetic alteration in prostate carcinogenesis, however, is DNA hypermethylation of specific CpG islands located near gene promoters, as reported for numerous genes including GSTP1, APC, and RASSF1A (for recent reviews see (1) and (4)). CpG island hypermethylation has been closely linked to gene silencing (5).

Methylation of DNA is a mechanism for changing the base sequence of DNA without altering its coding function. DNA methylation is a heritable, reversible and epigenetic change. DNA methylation harbours the potential to alter gene expression which in turn affects developmental and genetic processes. The methylation reaction involves flipping a target cytosine out of an intact double helix thereby allowing the transfer of a methyl group from S-adenosylmethionine in a cleft of the enzyme DNA (cystosine-5)-methyltransferase (Klimasauskas et al., Cell 76:357-369, 1994) to form 5-methylcytosine (5-mCyt). This enzymatic conversion is the only epigenetic modification of DNA known to exist in vertebrates and is essential for normal embryonic development (Bird, Cell 70:5-8, 1992; Laird and Jaenisch, Human Mol. Genet. 3:1487-1495, 1994; and Bestor and Jaenisch, Cell 69:915-926, 1992).

CpG-rich sequences are known as CpG islands. CpG islands are distributed across the human genome and often span the promoter region as well as the first exon of protein coding genes. Methylation of individual promoter region CpG islands usually turns off transcription by recruiting histone deacetylases, which supports the formation of inactive chromatin (2). CpG islands are typically between 0.2 to about 1 kb in length and are located upstream of many housekeeping and tissue-specific genes, but may also extend into gene coding regions. Therefore, it is the methylation of cytosine residues within CpG islands in somatic tissues, which is believed to affect gene function by altering transcription (Cedar, Cell 53:3-4, 1988).

Abnormal methylation of CpG islands associated with tumor suppressor genes may also cause decreased gene expression. Increased methylation of such regions may lead to progressive reduction of normal gene expression giving abnormal cells a growth advantage (i.e., a malignancy).

Methylation of promoter regions, particularly in tumour suppressor genes, and genes involved in apoptosis and DNA repair, is one of the hallmarks of cancer (2). Changes in the methylation status of these genes are an early event in cancer and continue throughout the different stages of the cancer. Specifically, distinct tumour types often have characteristic patterns of methylation, which can be used as markers for early detection and/or monitoring the progression of carcinogenesis. For therapeutic purposes, the methylation of certain genes, particularly DNA repair genes, can cause sensitivity to specific chemotherapeutics and methylation of those genes can thereby act as a predictive marker if those chemotherapeutic agents or treatment should be used.

SLC18A2 encodes vesicular monoamine transporter 2 (VMAT2), an integral membrane protein of secretory vesicles with predominant expression in neurons, neuroendocrine (NE) cells, and amine-handling hematopoietic cell types (6). It transports monoamines (dopamine, serotonin, epinephrine, norepinephrine, and histamine) from the cytosol into vesicles for storage and/or exocytotic release, e.g. during neurotransmission or auto-/paracrine factor release (6). SLC18A2 is also expressed in certain endocrine tumors, including NE prostate tumors (7), but rarely in non-endocrine cancers of the same tissues ((8, 9) and this study). Although murine knockout models have disclosed important biological roles of SLC18A2 in the nervous system (10-12), its possible function in normal and malignant prostate biology remains unknown. However, several of the monoamines that are substrates for SLC18A2-mediated transport have been shown to influence growth (13-15), proliferation (16), migration (17, 18) or apoptosis (19) of prostate cancer cells in vitro and in vivo.

As the PSA method is associated with significant false negative rates and does not distinguish well between clinically indolent or aggressive tumors (1), there is a need for novel markers of prostate cancer that can be used on their own, or in combination with existing markers, or other diagnostic and/or predictive methods, such as histopathological examination of biopsies.

In the present invention, SLC18A2 is disclosed as a novel marker of prostate cancer which can be used as an independent marker of prostate cancer, or in combination with established markers such as PSA, Gleason score, tumor stage, and surgical margin outcome. SLC18A2 is furthermore disclosed as a predictor of biochemical recurrence after radical prostatectomy.

### SUMMARY OF INVENTION

The present invention relates to a SLC18A2 gene as a marker of prostate cancer. The present invention thus relates to methods for diagnosing prostate cancer, predicting or prognosticating the disease outcome, for example recurrence following surgery, as well as methods for monitoring disease progression in an individual having contracted prostate cancer.

One aspect relates to a method for assisting in diagnosing and/or for diagnosing prostate cancer in an individual comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of presence or absence of prostate cancer.

An increased methylation status is indicative of the presence of prostate cancer, and similarly a decreased transcriptional and/or translational expression level in the sample is indicative of the presence of prostate cancer.

A second aspect of the present invention pertains to a method for assisting in prognosing and/or for prognosing the disease progression of prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of the progression of prostate cancer.

It is appreciated that an increased methylation status is indicative of disease progression of prostate cancer, and similarly a decreased transcriptional and/or translational expression level in the sample is indicative of disease progression of prostate cancer. Prognosis is used to predict the outcome or disease progression in the absence of intervention for example in the form of surgery.

Another aspect of the present invention relates to predicting the outcome of prostate cancer in an individual having contracted cancer. Prediction is here used to describe the outcome or disease progression following intervention. Thus, the present invention relates to a method for assisting in predicting and/or for predicting the outcome of prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of the outcome of prostate cancer.

It is appreciated that an increased methylation status is indicative of disease progression of prostate cancer, and similarly a decreased transcriptional and/or translational expression level in the sample is indicative of disease progression of prostate cancer.

A further aspect of the present invention relates to predicting the recurrence risk of prostate cancer in an individual having contracted cancer. Thus, claims are directed against a method for assisting in predicting and/or for predicting the recurrence risk of prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of the recurrence risk of prostate cancer.

It is appreciated that an increased methylation status is indicative of recurrence risk of prostate cancer, and similarly a decreased transcriptional and/or translational expression level in the sample is indicative of recurrence risk of prostate cancer.

Such recurrence risk is for example determined following radical prostatectomy, radiation therapy, cryotherapy or brachytherapy.

Yet another aspect of the present invention relates to a method for assisting in monitoring and/or for monitoring the effect of treatment on prostate cancer progression in an individual having contracted prostate cancer, comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of progression of said prostate cancer.

It is appreciated that an increased methylation status is indicative of the progression of prostate cancer, and similarly a decreased transcriptional and/or translational expression level in the sample is indicative of the progression of prostate cancer.

A further aspect pertains to methods for assisting in monitoring and/or for monitoring the progression of prostate cancer from a silent/indolent to an aggressive prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of the progression of prostate cancer from a silent/indolent to an aggressive prostate cancer.

A silent/indolent prostate cancer is a slow-growing and slow-progressing organ-confined prostate cancer with no or only minor clinical symptoms, whereas an aggressive prostate cancer is which has progressed or will progress relatively fast (i.e. within the remaining life expectancy of a given patient) to non-organ-confined prostate cancer.

Yet a further aspect of the present invention relates to a method for assisting in determining and/or determining the treatment regime of an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of the treatment regime to be offered to the individual having contracted prostate cancer.

Still another aspect of the present invention relates to a method of determining the status of an individual having or suspected of having prostate cancer. The method includes the steps of:
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of the status of said individual.

It is appreciated that the methods optionally comprises a step of comparing the methylation status of said SLC18A2 gene determined in the sample to the methylation status of a control sample, wherein the methylation status of said sample can be determined as increased or decreased.

The methylation status of said SLC18A2 gene in a sample being >1%, is indicative of an increased methylation level, wherein said sample is a tissue sample. However, when the methylation status of said SLC18A2 gene in a sample is >0% is indicative of an increased methylation status, wherein said sample is a urine sample. In one embodiment, the methylation status of said SLC18A2 gene in a sample is increased in comparison with a control sample, for example a fully unmethylated DNA fragment.

The methods of the present invention may optionally comprise a step of comparing the transcriptional and/or translational expression level of the SLC18A2 gene determined in the sample to the expression level of a control sample, wherein the expression level in the sample can be determined as increased or decreased.

The samples used in the methods are typically selected from tissue sample, blood, plasma, serum, semen, or urine. In one particular embodiment the sample is a biopsy of the prostate gland or resected prostate tissue following radical prostatectomy.

The methods of the present invention may further comprise a step of measuring the level of prostate specific antigen (PSA) in an individual.

Another aspect of the present invention relates to a method for treatment of an individual comprising the steps of
i) Selecting an individual having contracted prostate cancer, wherein the methylation status of the SLC18A2 gene is increased, and/or the transcriptional and/or translational expression level of the SLC18A2 gene is decreased, determined according to the methods herein
ii) Treating the individual with methylation modulating agent in a therapeutically effective amount to an individual in need thereof.

It is appreciated that the methylation modulating agent is capable of
i) reducing the methylation status of a SLC18A2 gene (SEQ ID NO: 1), or nucleotide sequence having at least 90% sequence identity with SEQ ID NO:1, or part thereof and/or
ii) increasing the transcriptional and/or translational expression level of the SLC18A2 gene.

The methylation modulating agent may be selected from the group consisting of DNA methylation inhibitors and histone deacetylases, such as 5-azacytidine (5-aza-CR), 5-aza-2′ deoxycytidine (5-aza-CdR), 5 fluorocytosine, pseudoisocytosine, Zebularine, Procainamide, polyphenol (−)-epigallocatechin-3-gallate (EGCG), Psammaplin, Trichostatin A, trapoxin B, depsipeptides, benzamides, electrophilic ketones, phenylbutyrate, sodium butyrate and valproic acid, but also suberoylanilide hydroxamic acid (SAHA/Vorinostat), Belinostat/PXD101, MS275, LAQ824/LBH589, 01994, MGCD0103, nicotinamide, derivatives of NAD, N-nitroso-n-methylurea, dihydrocoumarin, naphthopyranone, 4-phenylbutyric acid or 2-hydroxynaphaldehydes.

Yet a further aspect relates to a method for reducing tumorigenicity of a cell, said method comprising the steps of
i) providing
   a) at least one SLC18A2 gene (SEQ ID NO:1), or nucleotide sequence having at least 90% sequence identity with SEQ ID NO:1, or part thereof,
   b) at least one SLC18A2 gene transcript (SEQ ID NO: 2), or nucleotide sequence having at least 90% sequence identity with SEQ ID NO:2, or part thereof, and/or
   c) at least one translational product of the SLC18A2 gene (SEQ ID NO: 3), or nucleotide sequence having at least 90% sequence identity with SEQ ID NO:3, or part thereof
ii) introducing at least one of a), b), and/or c) of step i) into the tumor cell

One further aspect of the present invention pertains to a pharmaceutical composition for the treatment of prostate cancer comprising at least
i) at least one SLC18A2 gene (SEQ ID NO:1), or nucleotide sequence having at least 90% sequence identity with SEQ ID NO:1, or part thereof,
ii) ii) at least one SLC18A2 gene transcript (SEQ ID NO: 2), or nucleotide sequence having at least 90% sequence identity with SEQ ID NO:2, or part thereof, and/or
iii) iii) a translational product of the SLC18A2 gene (SEQ ID NO: 3), or nucleotide sequence having at least 90% sequence identity with SEQ ID NO:3, or part thereof.

Yet a further aspect relates to a kit comprising at least one detection member for a SLC18A2 gene, transcriptional and/or translational product or part thereof for use in the methods of the present invention. Such a detection member is an antibody directed against an epitope of the SLC18A2 protein or part thereof, oligonucleotides, primers and/or probes. The kit may further comprise means for providing the level and/or means for providing informations as to whether the level is above or below a cut off value.

A final aspect of the present invention relates to the use of an antibody directed against an epitope of the SLC18A2 protein or part thereof in the detection of the translational expression level of a SLC18A2 gene, or part thereof
i) for assisting in the diagnosis and/or for diagnosing of prostate cancer
ii) for assisting in the prognosis and/or for the prognosis of the disease progression of prostate cancer
iii) for assisting in the prediction and/or the prediction of the progression of prostate cancer
iv) for assisting in predicting and/or for predicting the recurrence risk of prostate cancer
v) for assisting in monitoring and/or for monitoring the effect of treatment on prostate cancer progression
vi) for assisting in monitoring and/or for monitoring the progression of prostate cancer from a silent to an aggressive prostate cancer
vii) for assisting in determining and/or determining the treatment regime

### DETAILED DESCRIPTION OF THE DRAWINGS

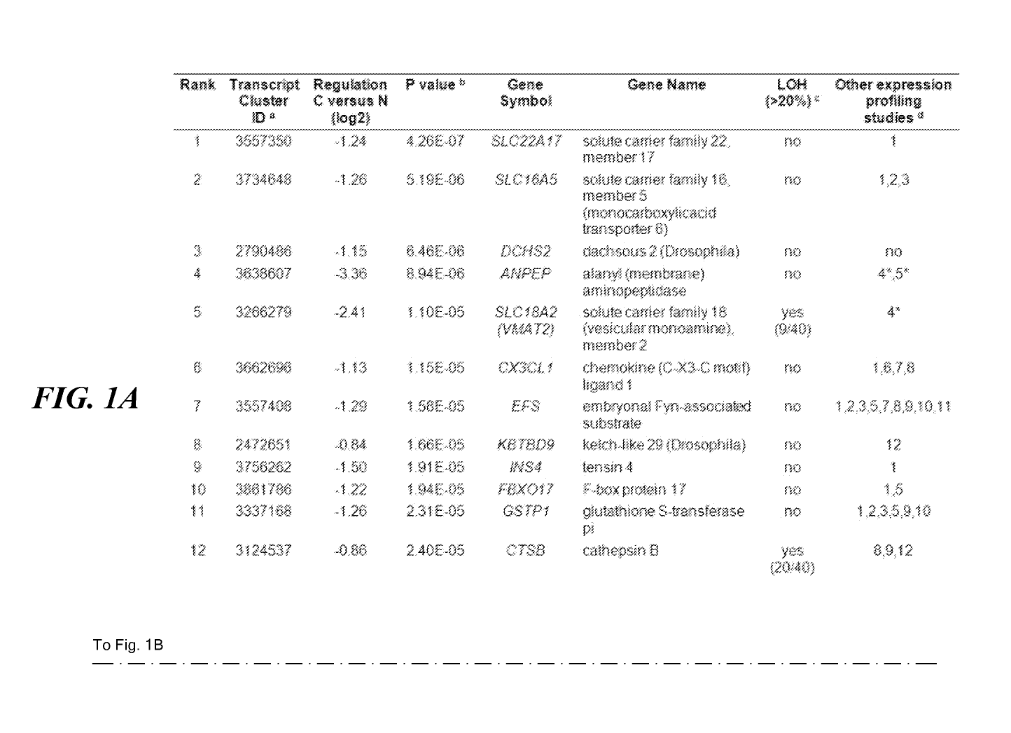

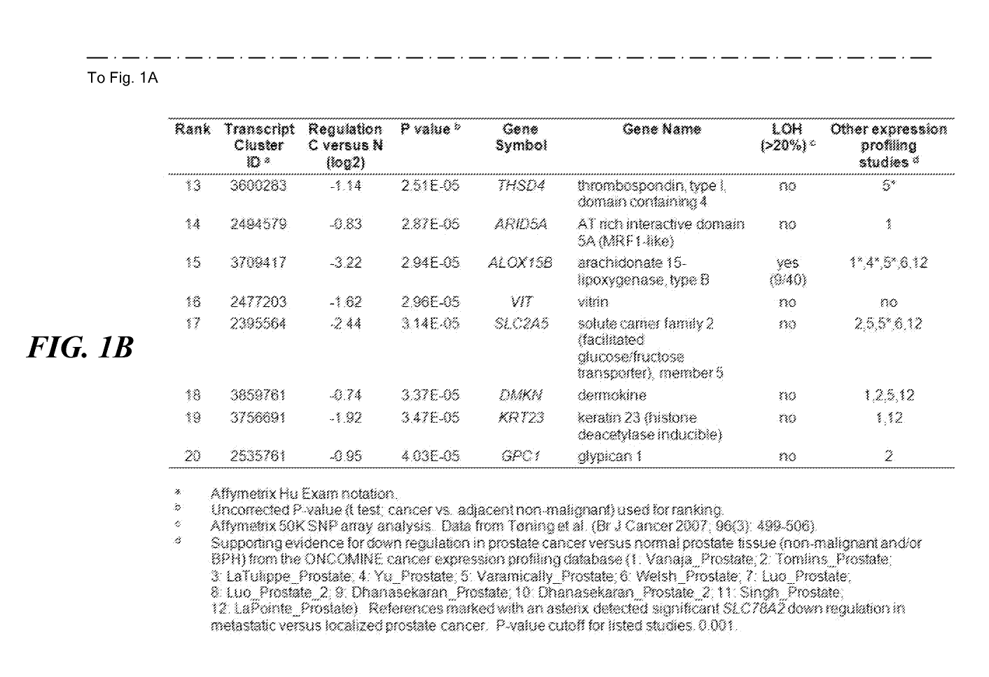

FIG. 1

Top 20 downregulated genes in prostate cancer (n=15) versus adjacent non-malignant prostate tissue (n=10) by exon array analysis

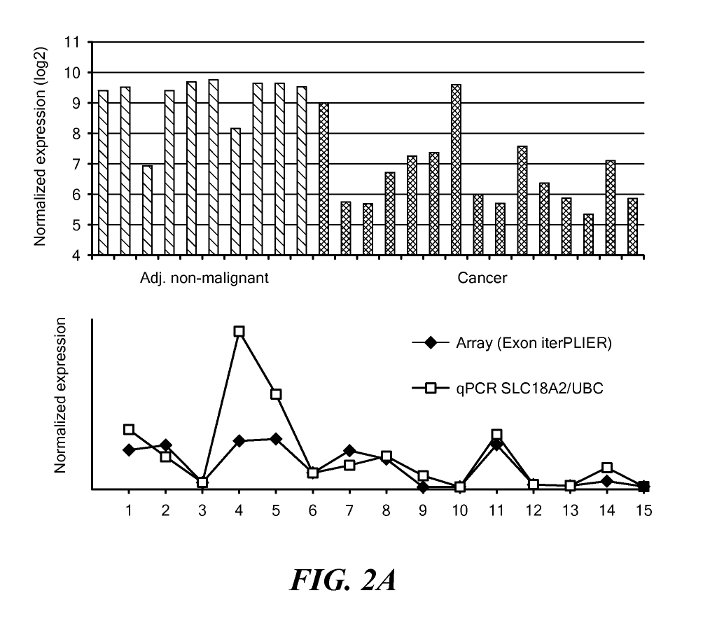

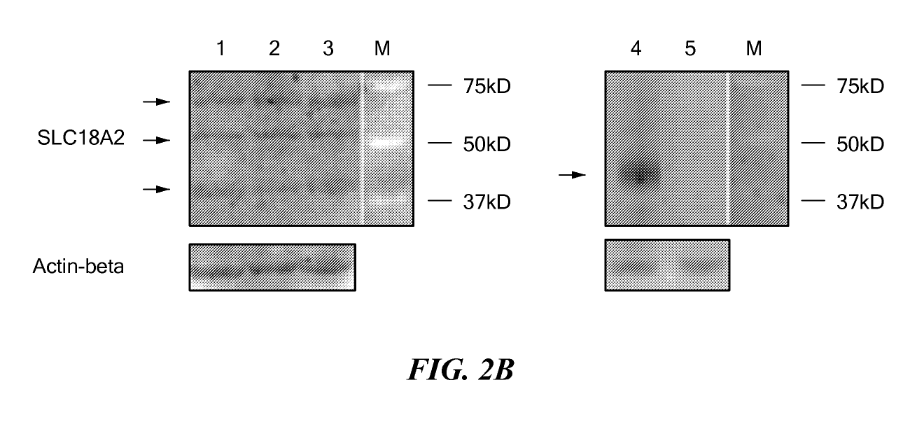

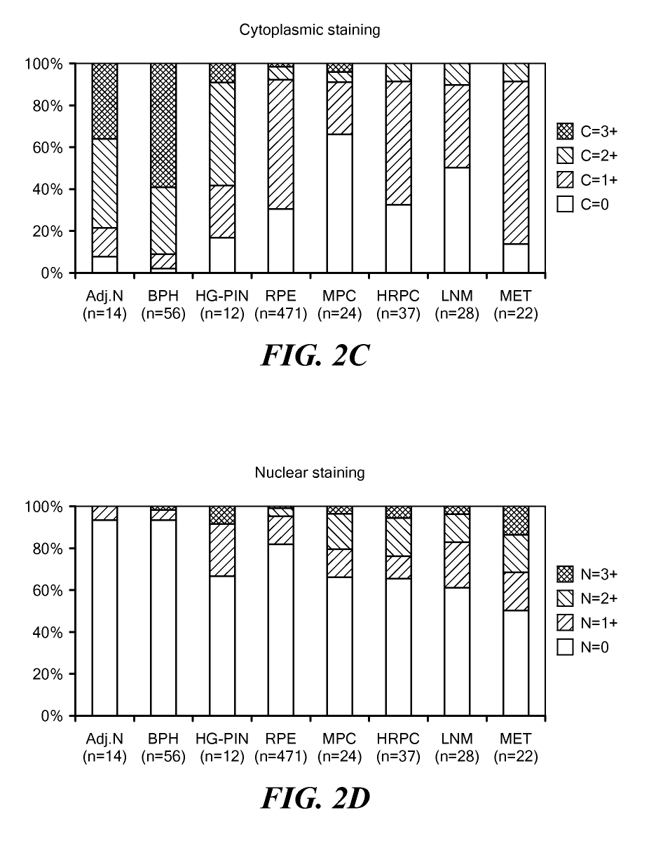

FIG. 2

A) Normalized SLC18A2 expression in adjacent non-malignant (grey bars) and prostate cancer tissue samples (black bars) determined by exon array analysis (top). Validation of SLC18A2 array-based expression levels (closed diamonds) by quantitative RT-PCR (open squares) on 15 randomly selected RNA samples (bottom). UBC was used for qRT-PCR normalization. The two methods correlated well (Pearson's correlation coefficient: 0.77).

B) Validation of SLC18A2 antibody specificity by Western blotting analysis of crude protein lysates from three BPH tissue samples (lanes 1-3) and COS7 cells transfected with an SLC18A2 (HA/Flag/His-tagged glycosylation mutant) expression plasmid (lane 4) or empty vector (lane 5). SLC18A2 immunoreactive bands are marked with arrows. The three bands in lanes 1-3 represent glycosylated (˜70 kDa), partially glycosylated (˜55 kDa), and unglycosylated (˜40 kDa) SLC18A2 protein. The slower migration of ectopic unglycosylated SLC18A2 in lane 4 is due to the internal HA tag and C-terminal Flag and His tags. Actin-beta was used as internal control. M, protein size marker.

C-D) Distribution of cytoplasmic and nuclear SLC18A2 staining patterns (white=no (0); light grey=weak (1+); dark grey=moderate (2+); black=strong (3+)) relative to tissue specimen types. The number of samples in each group is given in brackets. Adj.N, adjacent non-malignant tissue from patients with prostate cancer; BPH, benign prostatic hyperplasia; HG-PIN, high-grade prostatic intraepithelial neoplasia; RPE, prostate cancer tissue from radical prostatectomy patients with clinically localized disease; MPC, primary tumor from patients with metastatic prostate cancer; HRPC, hormone-refractory prostate cancer; LNM, lymph node metastasis; MET, distant metastasis (bone, lung or urinary bladder).

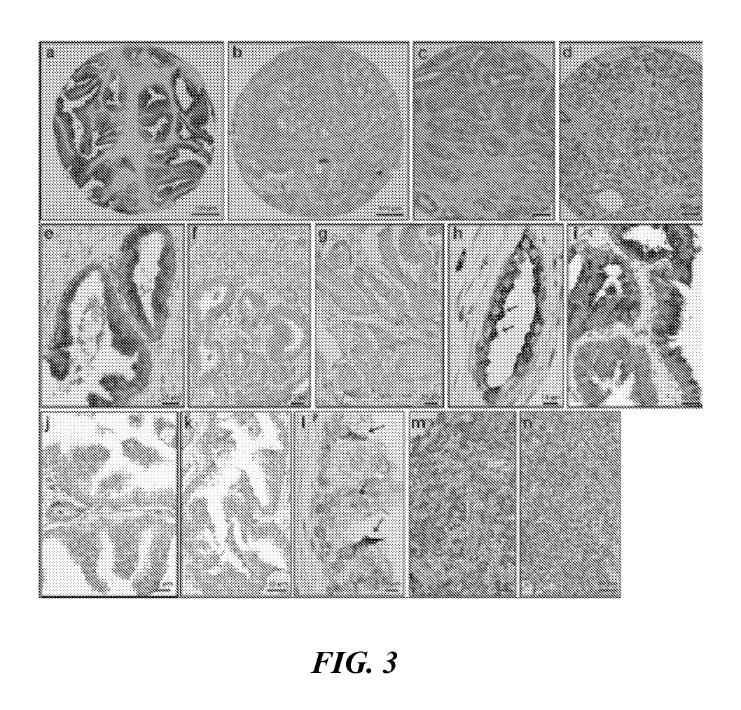

FIG. 3

IHC analysis of SLC18A2 expression on prostate cancer TMAs.

Panels a-d show representative examples of cores scored as C3+/N0 (cytoplasmic=3+/nuclear=0) (a), C0/N0 (b), C1+/N0 (c), and C0/N3+ (d). BPH with strong cytoplasmic staining (e). Prostate cancer specimens with no (f) and weak (g) cytoplasmic SLC18A2 staining. Panel h, high resolution image showing granular cytoplasmic staining in prostate epithelial cells. SLC18A2 staining is polarized towards the glandular lumen (marked by arrows). HG-PIN specimens showing strong (i), no (j), and weak (k) cytoplasmic SLC18A2 immunoreactivity. Prostate neuroendocrine cells positive for cytoplasmic SLC18A2 (l). Strong nuclear SLC18A2 staining in a primary tumor from a patient with metastatic prostate cancer (m) and in a lymph node metastasis (n).

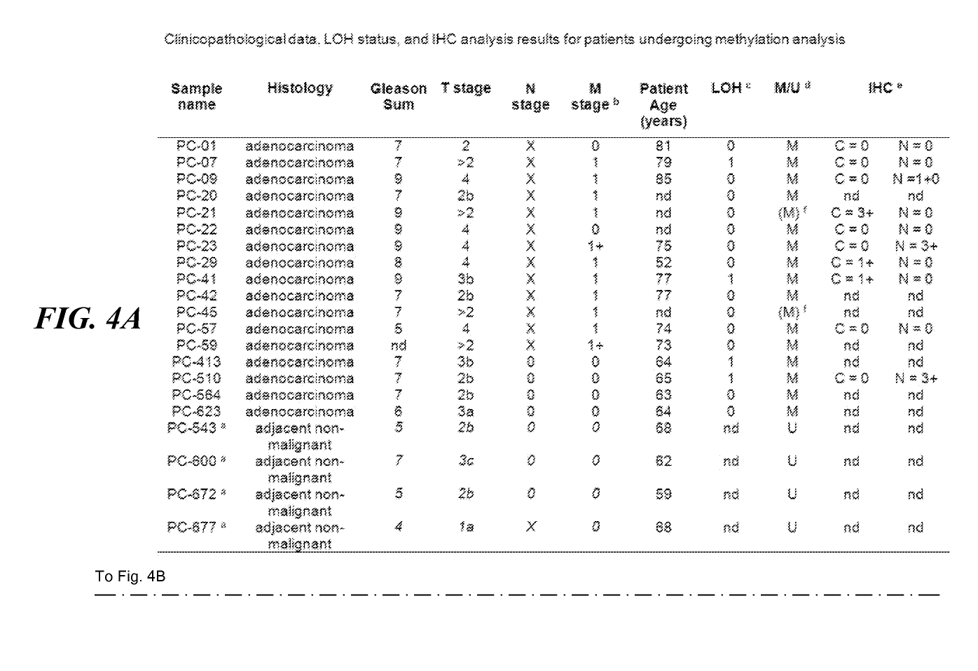

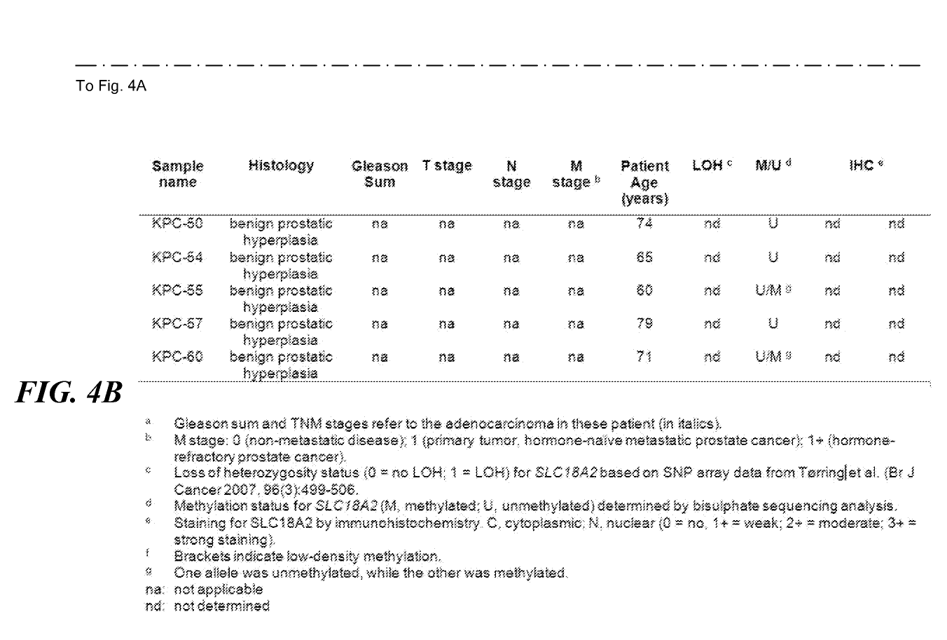

FIG. 4

Clinicopathological data, LOH status, and IHC analysis results for patients undergoing methylation analysis.

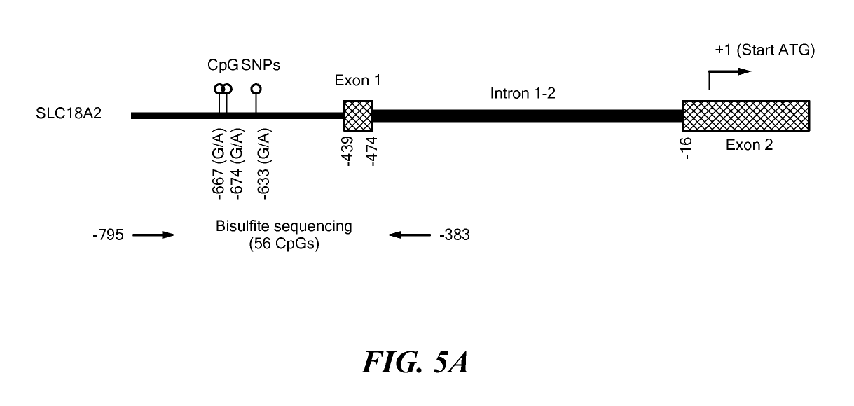

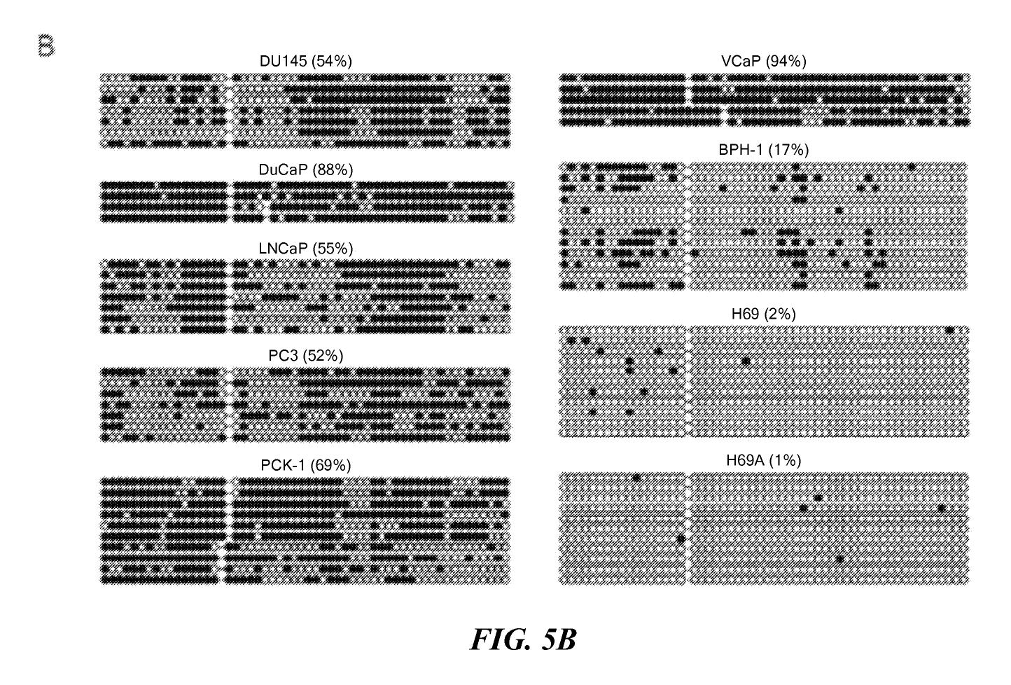

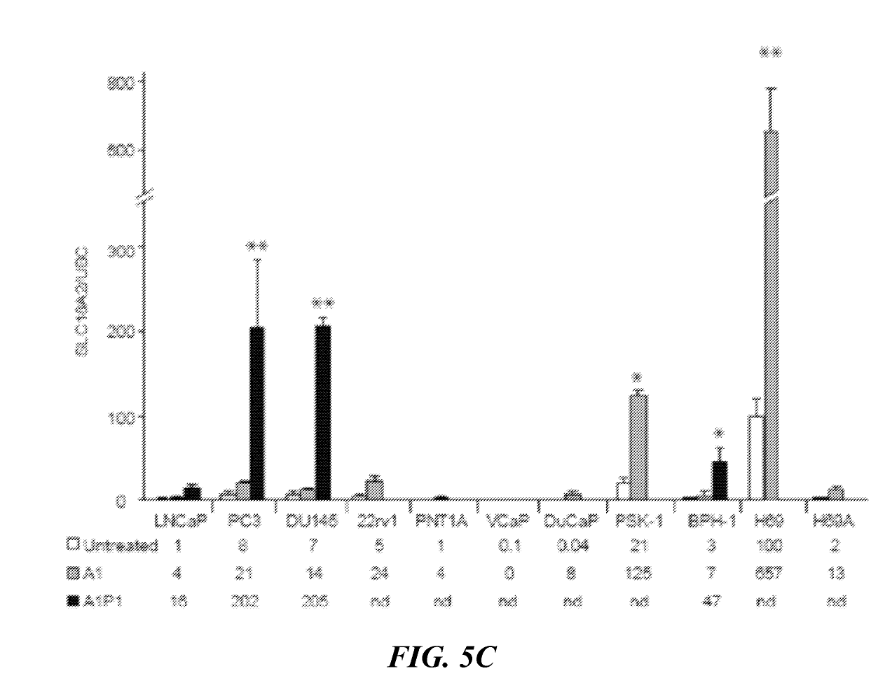

FIG. 5

A) genomic structure of the SLC18A2 gene locus. Locations of primers used for bisulfite sequencing PCR are shown below. The 412-bp amplified region contains 56 CpGs. Three of these coincide with SNPs and are marked with lollipops. Nucleotide positions relative to the translation start site (+1) are given. The first CpG interrogated by bisulfite sequencing is located at position −769 and the last at −418.

B) genomic bisulfite sequencing results. Open and closed circles represent unmethylated, respectively, methylated CpGs. Each line represents one clone. At least four clones were sequenced for each cell line. SNPs that disrupt CpGs are marked by horizontal lines. Average methylation percentages are given in brackets. C, normalized SLC18A2 expression in cell lines treated with 1 μM 5-aza-2′-dC alone (A1) or in combination with 1 mM 4-phenylbutyric acid (A1P1) as determined by real-time PCR. All reactions were run in triplicates. Error bars represent standard deviations of at least two independent experiments performed in duplicate. SLC18A2/UBC expression in H69 cells was arbitrarily set to 100. *p<0.005; **p<0.001 (2-sided t test). Mock treated cells (buffer only) had similar SLC18A2 expression as untreated cells (not shown).

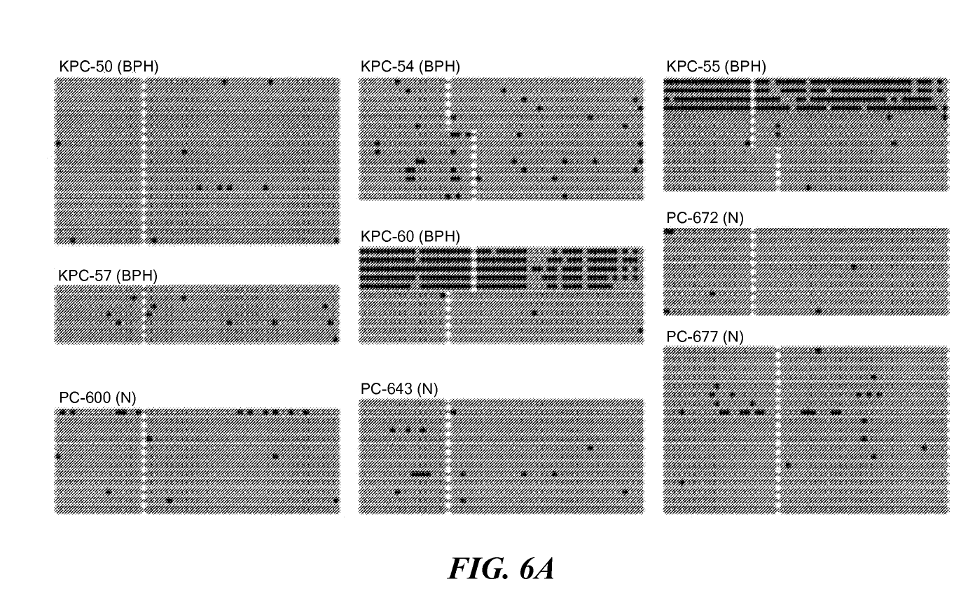

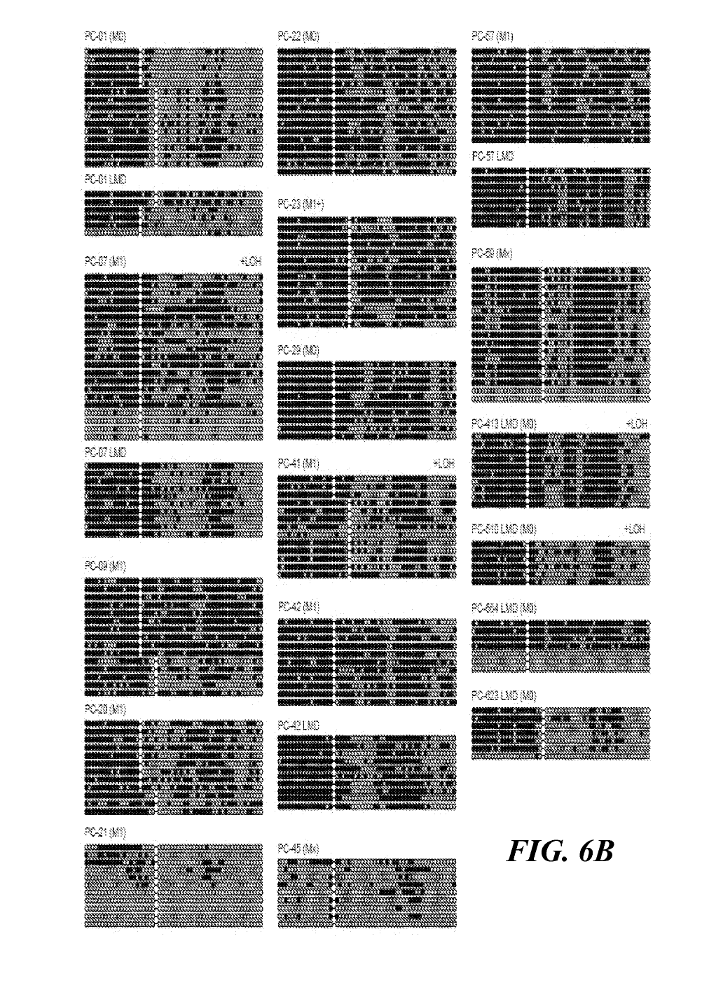

FIG. 6

A-B) SLC18A2 methylation patterns in non-malignant and prostate cancer tissue samples determined by bisulfite sequencing. Each line represents a single clone. Open and closed circles represent unmethylated and methylated CpGs, respectively. SNPs that disrupt CpGs are marked by horizontal lines. Bisulfite sequencing analysis of DNA extracted from laser-microdissected prostate adenocarcinoma cells was performed for 8 tumors (marked LMD). All other tissue samples were macrodissected. Four tumors showed loss of heterozygosity of SLC18A2 (marked by +LOH) by 50K SNP array analysis (31). All other tumors had no LOH at this locus. Similar hypermethylation patterns were seen in prostate tumors from M0 (no metastases) and M1 (with metastases) patients. N, adjacent non-malignant; Mx, unknown M status; M1+, M1 patient who has received anti-androgen therapy.

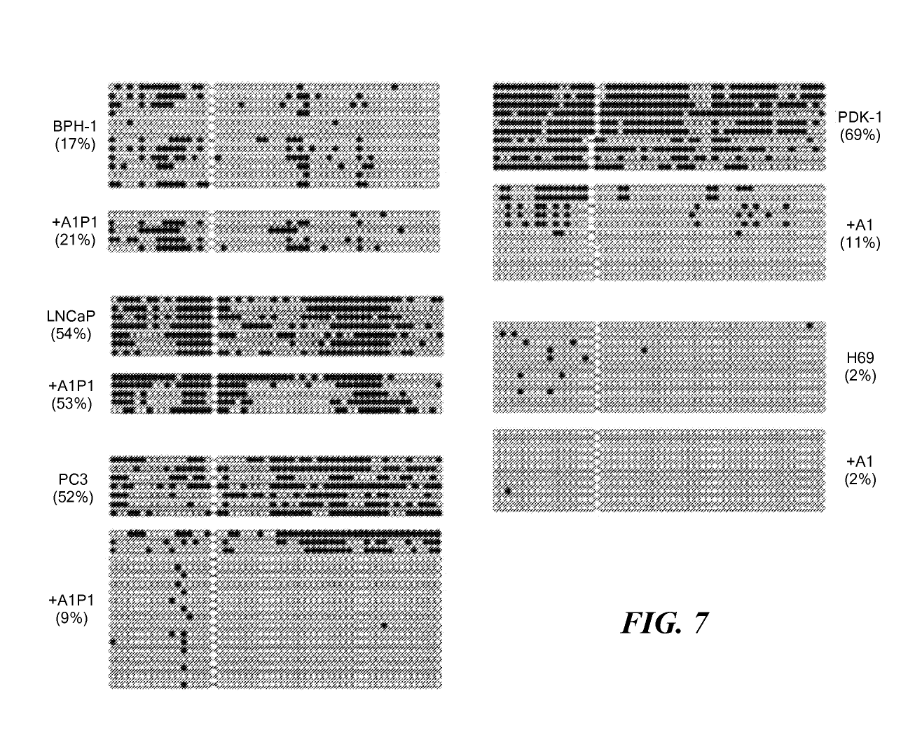

FIG. 7

Bisulfite sequencing results for SLC18A2 in cell lines before and after treatment with 1 microM 5-aza-2′-deoxycytidine alone (+A1) or in combination with 1 mM 4-phenylbutyric acid (+A1P1). Average percentages of methylated CpGs are given in brackets for each cell line and condition.

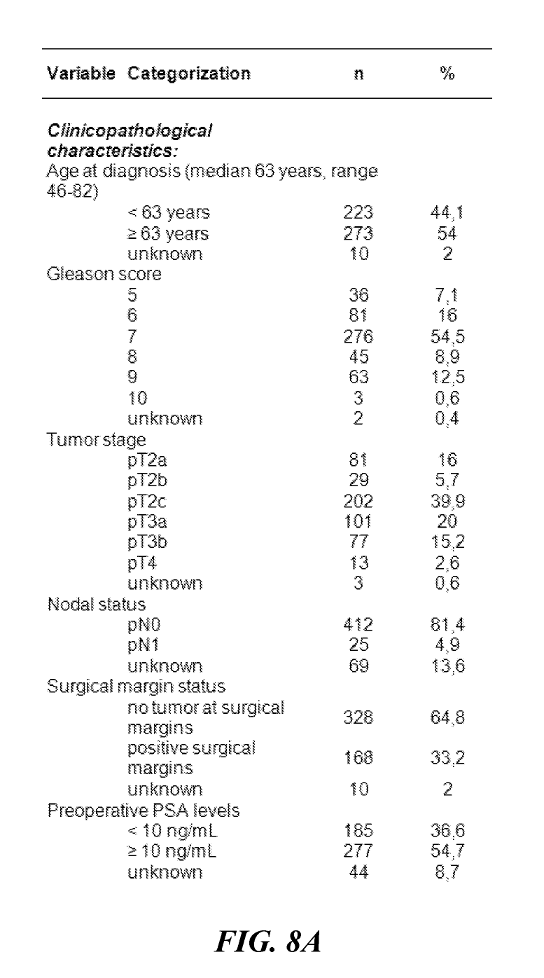

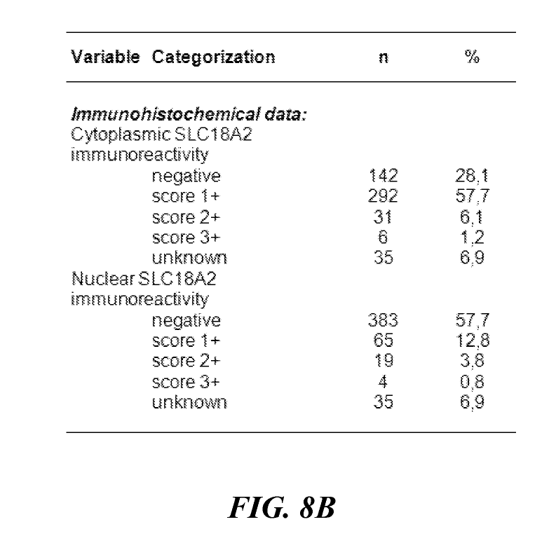

FIG. 8

Clinicopathological data and results of immunohistochemistry for patients receiving radical prostatectomy (n=506).

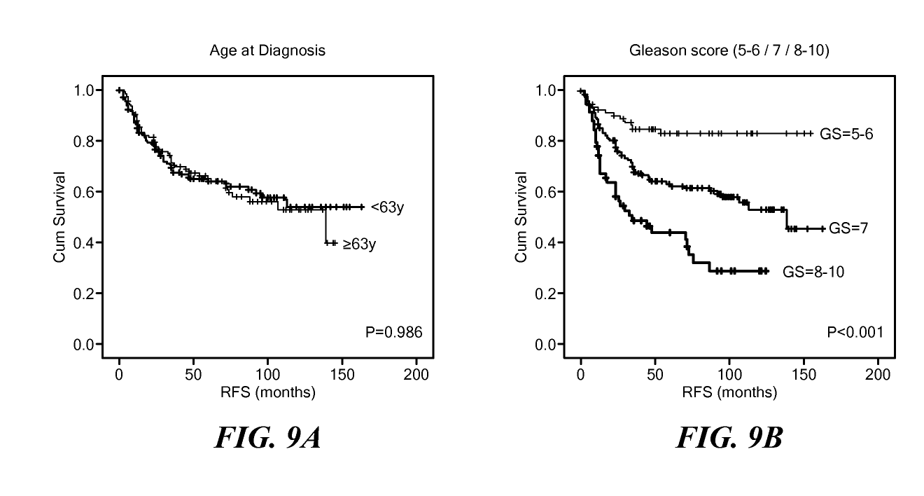

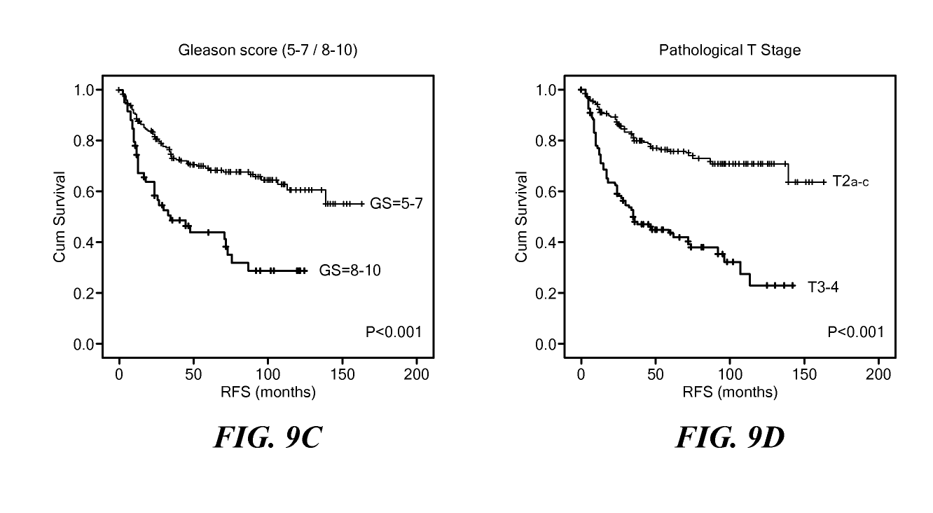

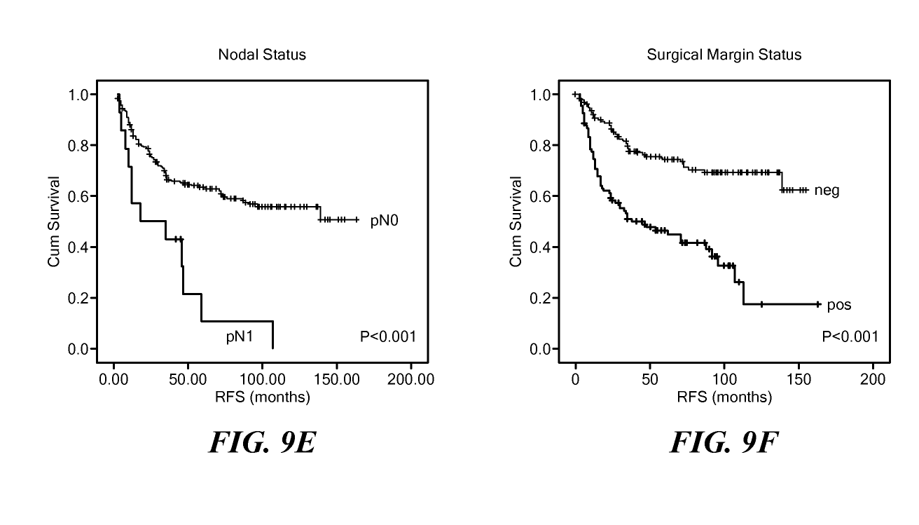

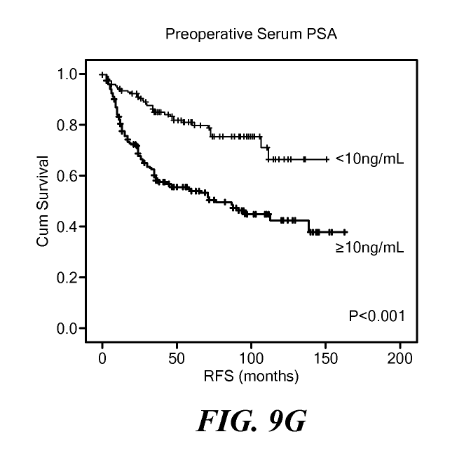

FIG. 9

Kaplan-Meier plots of biochemical recurrence-free survival (RFS) after radical prostatectomy in relation to age at diagnosis (A), Gleason score (B-C), pathological T stage (D), nodal status (E), surgical margin status (F), and preoperative serum PSA level (G). Two-sided log-rank test P-values are given for each plot. Significant P-values (<0.05) are in bold face.

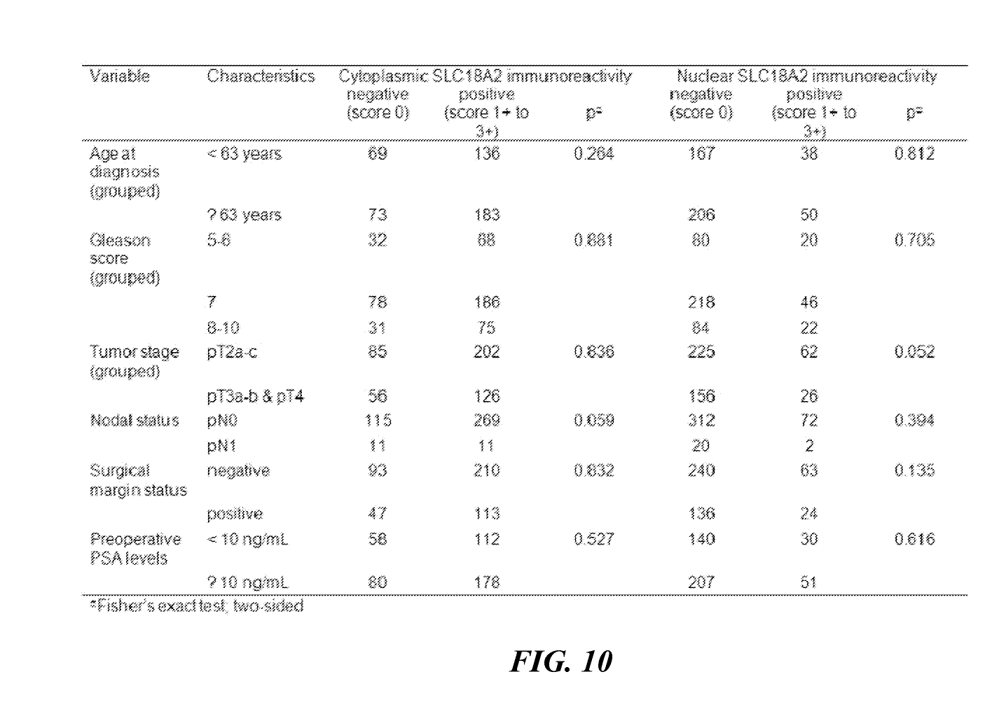

FIG. 10

Clinicopathological characteristics in relation to SLC18A2 immunoreactivity in radical prostatectomy (RPE) tumor specimens.

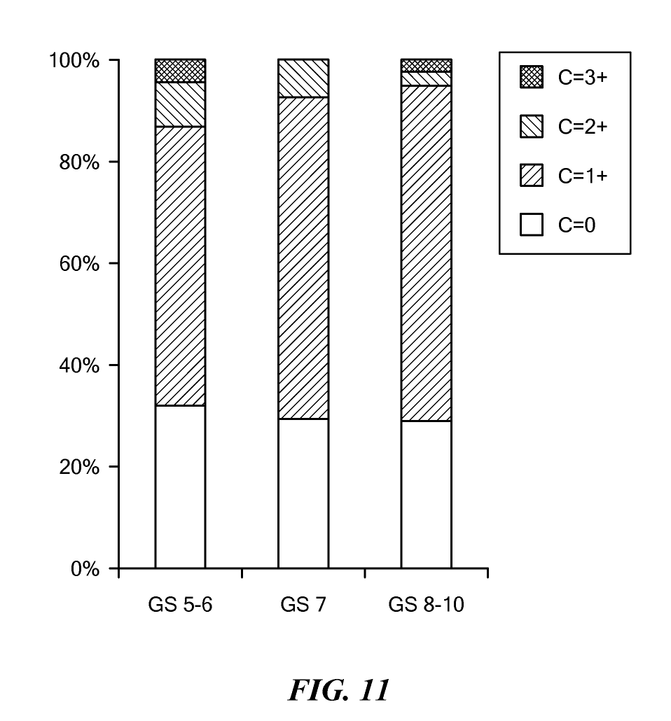

FIG. 11

Distribution of cytoplasmic SLC18A2 staining intensity scores (white=0; light grey=1+; dark grey=2+; black=3+) relative to Gleason score (GS).

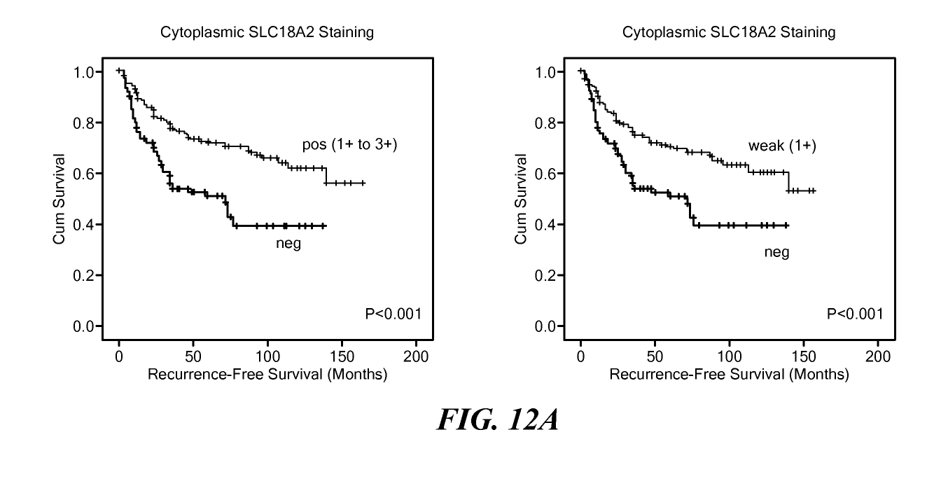

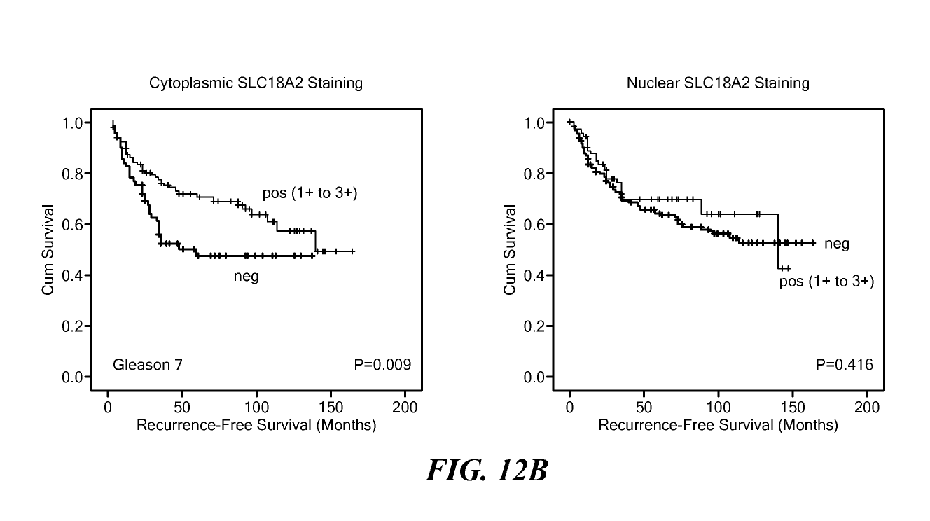

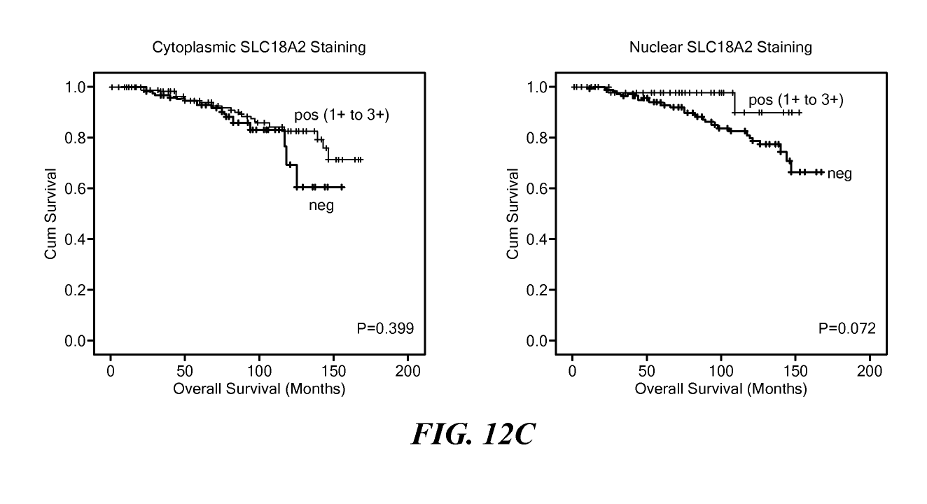

FIG. 12

Kaplan-Meier plots of recurrence-free survival and overall survival for radical prostatectomy patients. P-values for 2-sided log-rank statistics are given for each plot. A, RFS for patients with positive (intensity score=1+ to 3+) versus negative (intensity score=0) (left panel) or weak (intensity score=1+) versus negative (intensity score=0) cytoplasmic SLC18A2 staining (right panel). B, RFS for the subgroup of patients with Gleason 7 tumors, regarding cytoplasmic SLC18A2 immunoreactivity (left panel), and for patients with positive (intensity score=1+ to 3+) versus negative (intensity score=0) nuclear SLC18A2 staining (right panel). C, overall survival curves for patients with or without cytoplasmic (left panel) or nuclear (right panel) SLC18A2 immunoreactivity.

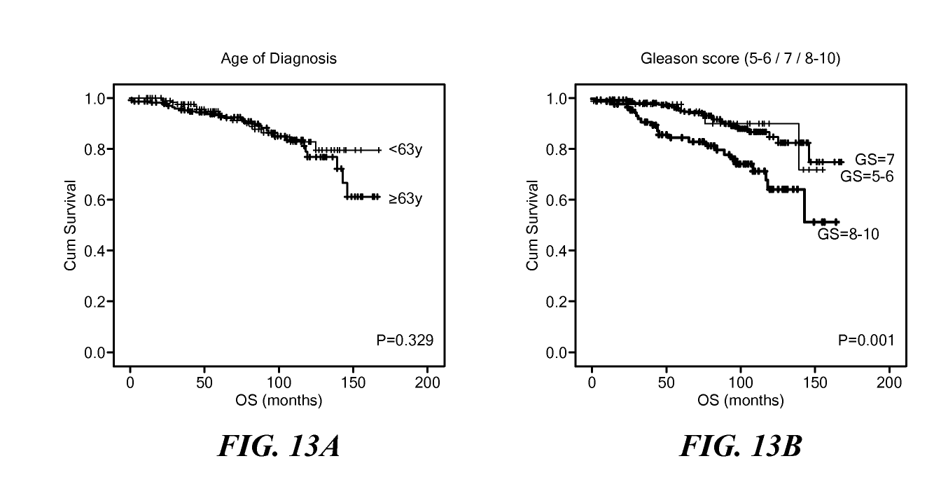

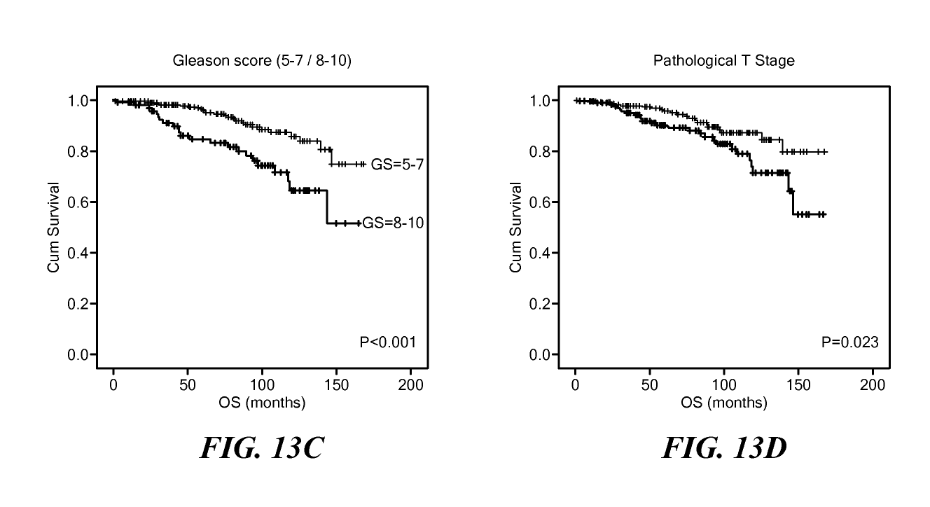

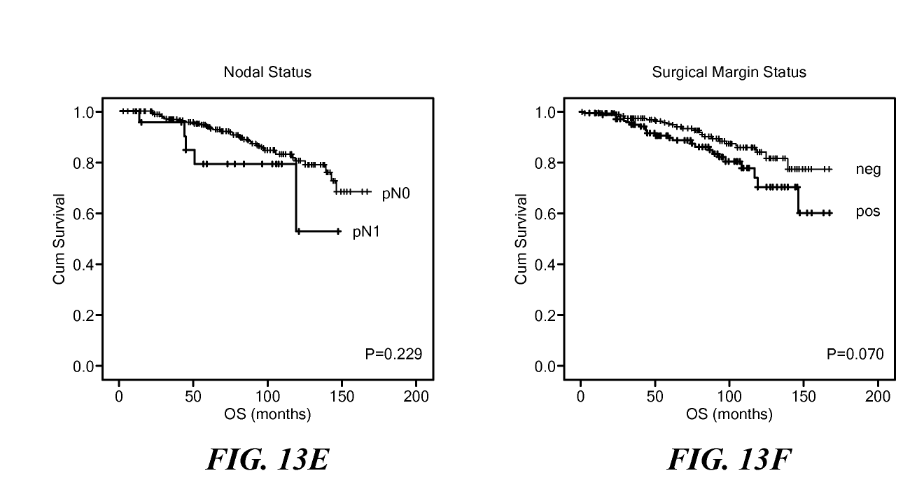

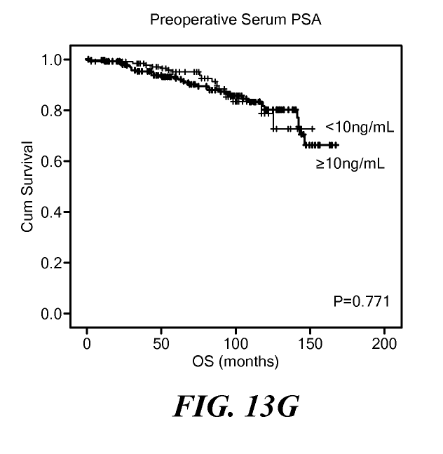

FIG. 13

Kaplan-Meier plots of biochemical overall survival (OS) after radical prostatectomy in relation to age at diagnosis (A), Gleason score (B-C), pathological T stage (D), nodal status (E), surgical margin status (F), and preoperative serum PSA level (G). Two-sided log-rank test P-values are given for each plot. Significant P-values (<0.05) are in bold face.

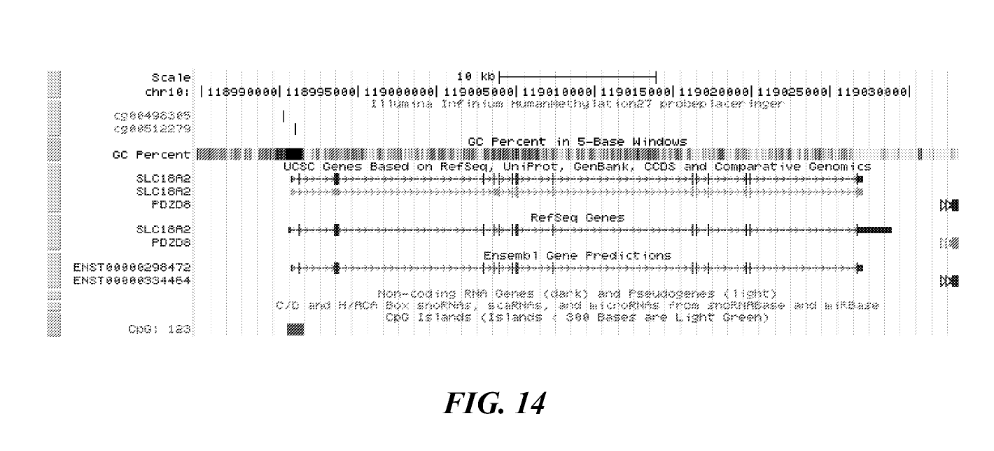

FIG. 14

Genomic location of probe “cg00498305” and “cg00512279” Infinium HumanMethylation27 BeadChip (Illumina). Probe “cg00498305” interrogates a CpG site at position −969 upstream of the ATG site. Probe “cg00512279” interrogates a CpG site at position −214 upstream of the ATG site.

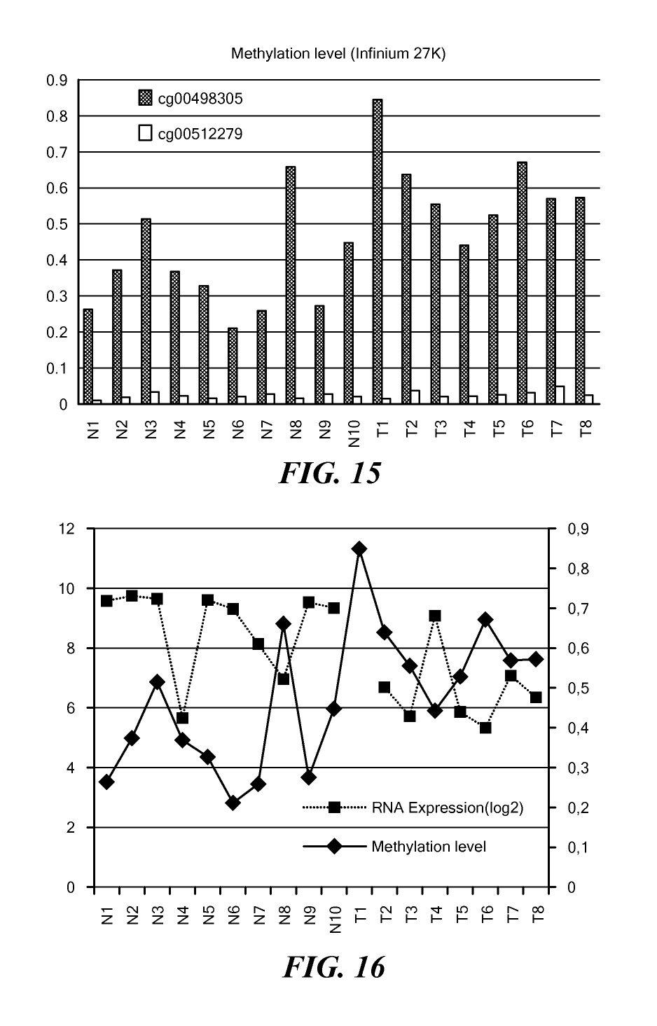

FIG. 15

DNA methylation of the CpG sites located at −969 base pairs and −214 base pairs of SLC18A2 in non-malignant and prostate cancer tissue. N1-10: Adjacent non-malignant prostate tissue samples; T1-8: Prostate cancer tissue samples. Black bars indicate DNA methylation of the CpG sites located at −969 base pairs, detected by the probe cg00498305. Grey bars indicate DNA methylation of the CpG sites located at −214 base pairs, detected by the probe cg00512279. The methylation was determined using Infinium HumanMethylation27 BeadChip (Illumina).

FIG. 16

Data showing a clear inverse correlation between SLC18A2 transcript levels (determined by Affymetrix Exon Array analysis; curve marked with squares) and methylation of SLC18A2 at position −969 (determined by Illumina Infunium 27K BeadChip analysis; probe “cg00498305”; curve marked with diamonds) in clinical prostate cancer tissue samples

### DETAILED DESCRIPTION OF THE INVENTION

Priority is claimed to U.S. Provisional Application No. 61/181,326, filed May 27, 2009, which is hereby incorporated by reference in its entirety. All patent and non-patent references cited herein are also hereby incorporated by reference in their entirety.

Epigenetic changes, resulting from DNA and histone modifications, may lead to heritable silencing of genes without a change in their coding sequence. These changes are usually established in parental germ cells and inherited post fertilization to the offspring during successive cell divisions.

The most prominent DNA epigenetic modification is by methylation typically on CpG islands. These are sequence regions of more than 500 base pairs in size with a GC content greater than 55%, normally kept free of DNA methylation and are the sites of DNA methylation in various conditions or pathologies. CpG islands are located within the promoter regions of about 40% of mammalian genes and when methylated, cause gene silencing. Epigenetic changes may also occur on chromatin. Chromatin modifications such as histone acetylation, deacetylation, methylation or demethylation of conserved lysine residues on the amino-terminal tail domains are associated with transcriptional activation or silencing.

The present invention discloses the SLC18A2 gene as a marker of prostate cancer. The present inventors have found that the SLC18A2 is a common target for promoter hypermethylation in prostate cancer. Thus, the methylation status of SLC18A2 can be used as a novel tool for assisting in the diagnosis of prostate cancer presence or absence, as well as for assisting in the prediction and/or prognosis of the disease progression in an individual having contracted cancer. Furthermore, it is disclosed that the transcriptional and/or translational expression level of the SLC18A2 gene can further be used for assisting in the diagnosis of prostate cancer presence or absence, aswell as for assisting in the prediction and/or prognosis of the disease progression in an individual having contracted cancer.

### DEFINITIONS

The terms ‘prostate cancer’ is herein used interchangeably with and is equivalent to the term ‘prostate adenocarcinoma’.

The terms ‘diagnosis, diagnostic, diagnosing’ are used herein as terms for the act of determining the nature and cause of a disease for example through evaluation of patient history, examination, and review of laboratory data.

The term ‘assisting’ as used to describe the methods herein is to emphasise that the methods herein may be directed solely to the step of gathering data to assist in the diagnosis, prediction, and prognosis of prostate cancer. Accordingly, methods for assisting in such methods are not to be construed as diagnostic methods practiced on the human body.

The terms ‘prognosis, prognostic, prognosing’ are used herein as terms for predicting the outcome or disease progression independent of intervention for example in the form of no treatment, surgery, medication etc.

The terms ‘prediction, predictive, predicting’ are used herein as terms for predicting the outcome or disease progression following intervention for example in the form of surgery, medication etc. For example such an intervention may be radical prostatectomy.

The term “treatment”, as used herein comprises any type of therapy, which aims at terminating, preventing, ameliorating and/or reducing the susceptibility to prostate cancer.

In a preferred embodiment, the term treatment relates to prophylactic treatment, i.e. a therapy to reduce the susceptibility of prostate cancer.

The term ‘individual’ and individual in need thereof' as used herein refers to a male mammal, preferably a male human being at any age which is suspected of having prostate cancer, is predisposed to develop prostate cancer or has contracted prostate cancer.

The terms ‘therapeutically effective amount’ means an amount that is sufficient to elicit a desired response.

The term ‘gene’ as used herein refers to its normal meaning, a nucleic acid sequence with a transcriptional capability, i.e., which can be transcribed into an RNA sequence (an expressed sequence) which in most cases, is translated into an amino acid sequence, along with the regulatory sequences that regulate expression or engage in the expression of expressed sequences.

A double stranded polynucleotide contains two strands that are complementary in sequence and capable of hybridizing to one another.

A nucleotide is herein defined as a monomer of RNA or DNA. A nucleotide is a ribose or a deoxyribose ring attached to both a base and a phosphate group. Both mono-, di-, and tri-phosphate nucleosides are referred to as nucleotides.

The term ‘nucleotides’ as used herein refers to both natural nucleotides and non-natural nucleotides capable of being incorporated—in a template-directed manner—into an oligonucleotide, preferably by means of an enzyme comprising DNA or RNA dependent DNA or RNA polymerase activity, including variants and functional equivalents of natural or recombinant DNA or RNA polymerases. Corresponding binding partners in the form of coding elements and complementing elements comprising a nucleotide part are capable of interacting with each other by means of hydrogen bonds. The interaction is generally termed “base-pairing”. Nucleotides may differ from natural nucleotides by having a different phosphate moiety, sugar moiety and/or base moiety. Nucleotides may accordingly be bound to their respective neighbour(s) in a template or a complementing template by a natural bond in the form of a phosphodiester bond, or in the form of a non-natural bond, such as e.g. a peptide bond as in the case of PNA (peptide nucleic acids). Nucleotides according to the invention includes ribonucleotides comprising a nucleobase selected from the group consisting of adenine (A), uracil (U), guanine (G), and cytosine (C), and deoxyribonucleotide comprising a nucleobase selected from the group consisting of adenine (A), thymine (T), guanine (G), and cytosine (C). Nucleobases are capable of associating specifically with one or more other nucleobases via hydrogen bonds. Thus it is an important feature of a nucleobase that it can only form stable hydrogen bonds with one or a few other nucleobases, but that it can not form stable hydrogen bonds with most other nucleobases usually including itself. The specific interaction of one nucleobase with another nucleobase is generally termed “base-pairing”. The base pairing results in a specific hybridisation between predetermined and complementary nucleotides.

Complementary nucleotides according to the present invention are nucleotides that comprise nucleobases that are capable of base-pairing. Of the naturally occurring nucleobases adenine (A) pairs with thymine (T) or uracil (U); and guanine (G) pairs with cytosine (C). Accordingly, e.g. a nucleotide comprising A is complementary to a nucleotide comprising either T or U, and a nucleotide comprising G is complementary to a nucleotide comprising C.

The term ‘oligonucleotide’ is used herein interchangebly with polynucleotide. As used herein the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions (see disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050). The term oligonucleotide thus also refers to any combination of oligonucleotides of natural and non-natural nucleotides. The natural and/or non-natural nucleotides may be linked by natural phosphodiester bonds or by non-natural bonds. Preferred oligonucleotides comprise only natural nucleotides linked by phosphodiester bonds. Oligonucleotide is used interchangeably with polynucleotide. The oligomer or polymer sequences of the present invention are formed from the chemical or enzymatic addition of monomer subunits. The term “oligonucleotide” as used herein includes linear oligomers of natural or modified monomers or linkages, including deoxyribonucleotides, ribonucleotides, anomeric forms thereof, peptide nucleic acid monomers (PNAs), locked nucleotide acid monomers (LNA), and the like, capable of specifically binding to a single stranded polynucleotide tag by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′→3′ order from left to right and the “A” denotes deoxyadenosine, “C” denotes deoxycytidine, “G” denotes deoxyguanosine, and “T” denotes thymidine, unless otherwise noted. Usually oligonucleotides of the invention comprise the four natural nucleotides; however, they may also comprise methylated or non-natural nucleotide analogs. Suitable oligonucleotides may be prepared by the phosphoramidite method described by Beaucage and Carruthers (Tetrahedron Lett., 22, 1859-1862, 1981), or by the triester method according to Matteucci, et al. (J. Am. Chem. Soc., 103, 3185, 1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical configuration typically associated with, for example, DNA. In addition to the 100% complementary form of double-stranded oligonucleotides, the term “double-stranded” as used herein is also meant to refer to those forms which include such structural features as bulges and loops. For example as described in U.S. Pat. No. 5,770,722 for a unimolecular double-stranded DNA. It is clear to those skilled in the art when oligonucleotides having natural or non-natural nucleotides may be employed, e.g. where processing by enzymes is called for, usually oligonucleotides consisting of natural nucleotides are required. When nucleotides are conjugated together in a string using synthetic procedures, they are always referred to as oligonucleotides.

A plurality of individual nucleotides linked together in a single molecule may form a polynucleotide. Polynucleotide covers any derivatized nucleotides such as DNA, RNA, PNA, LNA etc. Any oligonucleotide is also a polynucleotide, but every polynucleotide is not an oligonucleotide.

The term “dinucleotide” as used herein refers to two sequential nucleotides. The dinucleotide may be comprised in an oligonucleotide or a polynucleotide. In particular, the dinucleotide CpG, which denotes a cytosine linked to a guanine by a phosphodiester bond, may be comprised in an oligonucleotide according to the present invention. A CpG dinucleotide is also herein referred to as a CpG site.

Methylation status: the term “methylation status” as used herein, refers to the presence or absence of DNA methylation. The methylation status of a given DNA sample is given as the ratio of methylated versus methylated and non-methylated allelles for either an individual CpG dinucleotide or for a long stretch of DNA sequence comprising at least two CpG dinucleotides, such as 3, 4, 5, 6, 7, 8, 9, 10, 15, 20, 30, 40 CpG dinucleotides.

### Expression Level

By the term transcriptional expression level is meant the level of SLC18A2 transcripts, variants or nucleotide sequence having at least 90% sequence identity with SEQ ID NO:2, thereof or part thereof, in a given sample, for example in a tissue sample, or a urine sample. By the term translational expression level is meant the level of SLC18A2 protein, or amino acid sequence having at least 90% sequence identity with SEQ ID NO:3, or part thereof, as described elsewhere herein, in a given sample, for example in a tissue sample, or a urine sample. The level of SLC18A2 protein can be determined by for example immunohistochemistry or other methods using antibodies that specifically recognize SLC18A2 protein or fragment thereof. The level of SLC18A2 transcript is determined by for example quantitative RT_PCR with normalisation to at least one relevant other gene.

The term “Test Sensitivity” is the ability of a screening test to identify true disease, also characterised by being a test with high sensitivity has few false negatives, additionally a test independent of disease prevalence. The test sensitivity is calculated as true positive tests per total affected patients tested, expressed as a percentage.

The term “Test Specificity” is a screening test which is correctly negative in the absence of disease, has high specificity and few false positives, is independent of disease prevalence. The test specificity is calculated as true negative tests per unaffected individual s tested, expressed as a percentage.

The term “PPV” (Positive Predictive Value) is the percent of patients with positive test having disease, and thus assesses reliability of positive test. Calculation: PPV=(True positive)/(True+False positives)

The term “NPV” (Negative Predictive Value) refers to patients with negative test that do not have disease, and assesses reliability of negative test. Calculation: NPV=(True negative)/(true and false negatives).

Amino acid: Entity comprising an amino terminal part (NH₂) and a carboxy terminal part (COOH) separated by a central part comprising a carbon atom, or a chain of carbon atoms, comprising at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed in Table 2 herein below. Non-natural amino acids are those not listed in Table 2. Examples of non-natural amino acids are those listed e.g. in 37 C.F.R. section 1.822(b)(4), all of which are incorporated herein by reference. Further examples of non-natural amino acids are listed herein below. Amino acid residues described herein can be in the “D” or “L” isomeric form.

TABLE 2
Natural amino acids and their respective codes.

| Symbols | | |
|---|---|---|
| 1-Letter | 3-Letter | Amino acid |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

Amino acid residue: the term “amino acid residue” is meant to encompass amino acids, either standard amino acids, non-standard amino acids or pseudo-amino acids, which have been reacted with at least one other species, such as 2, for example 3, such as more than 3 other species. In particular amino acid residues may comprise an acyl bond in place of a free carboxyl group and/or an amine-bond and/or amide bond in place of a free amine group. Furthermore, reacted amino acids residues may comprise an ester or thioester bond in place of an amide bond

The term ‘stringent conditions’ as used herein shall denote stringency as normally applied in connection with Southern blotting and hybridization as described e.g. by Southern E. M., 1975, J. Mol. Biol. 98:503-517. For such purposes it is routine practise to include steps of prehybridization and hybridization. Such steps are normally performed using solutions containing 6×SSPE, 5% Denhardt's, 0.5% SDS, 50% formamide, 100 μg/ml denatured salmon testis DNA (incubation for 18 hrs at 42° C.), followed by washings with 2×SSC and 0.5% SDS (at room temperature and at 37° C.), and a washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

Functional equivalents and variants are used interchangeably herein. In one preferred embodiment of the invention there is also provided variants of SLC18A2 gene variants of fragments thereof.

When being polypeptides, variants are determined on the basis of their degree of identity or their homology with a predetermined amino acid sequence, said predetermined amino acid sequence being of SEQ ID NO: 3, when the variant is a fragment, a fragment of any of the aforementioned amino acid sequences, respectively. SEQ ID NO: 3 is the amino acid sequence of the SLC18A2 protein (accession number NP_{—}003045). Accordingly, variants preferably have at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence SEQ ID NO:3.

Sequence identity is determined in one embodiment by utilising fragments of SEQ ID NO:3 peptides comprising at least 25 contiguous amino acids and having an amino acid sequence which is at least 80%, such as 85%, for example 90%, such as 95%, for example 99% identical to the amino acid sequence of SEQ ID NO: 3, wherein the percent identity is determined with the algorithm GAP, BESTFIT, or FASTA in the Wisconsin Genetics Software Package Release 7.0, using default gap weights.

As used herein “variant” refers to polypeptides or proteins which are homologous to the SLC18A2 protein, (SEQ ID NO.: 3), i.e. the translational product of the SLC18A2 gene, but which differs from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide.

A person skilled in the art will know how to make and assess ‘conservative’ amino acid substitutions, by which one amino acid is substituted for another with one or more shared chemical and/or physical characteristics. Conservative amino acid substitutions are less likely to affect the functionality of the protein. Amino acids may be grouped according to shared characteristics. A conservative amino acid substitution is a substitution of one amino acid within a predetermined group of amino acids for another amino acid within the same group, wherein the amino acids within a predetermined groups exhibit similar or substantially similar characteristics.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine, a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Substitutions within the following groups (‘strong’ conservation group) are to be regarded as conservative substitutions within the meaning of the present invention
STA, NEQK, NHQK, NDEQ, QHRK, MILV, MILF, HY, FYW.

Substitutions within the following groups (‘weak’ conservation group) are to be regarded as semi-conservative substitutions within the meaning of the present invention
CSA, ATV, SAG, STNK, STPA, SGND, SNDEQK, NDEQHK, NEQHRK, VLIM, HFY.

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

Suitably variants are variant(s) of SEQ ID NO: 3 having at least 80% sequence identity, such as preferably at least 81% sequence identity, more preferably e.g. at least 82% sequence identity, such as more preferably at least 83% sequence identity, e.g. more preferably at least 84% sequence identity, more preferably such as at least 85% sequence identity, more preferably e.g. at least 86% sequence identity, more preferably such as at least 87% sequence identity, more preferably e.g. at least 88% sequence identity, more preferably such as at least 89% sequence identity, more preferably e.g. at least 90% sequence identity, more preferably such as at least 91% sequence identity, more preferably e.g. at least 92% sequence identity, such as at least 93% sequence identity, more preferably e.g. at least 94% sequence identity, more preferably such as at least 95% sequence identity, more preferably e.g. at least 96% sequence identity, more preferably such as at least 97% sequence identity, more preferably e.g. at least 98% sequence identity, more preferably such as at least 99% sequence identity, more preferably e.g. at least 99.5% sequence identity with the predetermined sequence of SLC18A2 (SEQ ID No: 3).

The fragment of the translational product of the SLC18A2 gene according to the invention, wherein the fragment has a stretch of at least 50 contiguous amino acids contains less than 450 consecutive amino acid residues of SEQ ID NO: 3, such as less than 440 consecutive amino acid residues, e.g. less than 430 consecutive amino acid residues, such as less than 420 consecutive amino acid residues, such as less than 410 consecutive amino acid residues, such as less than 400 consecutive amino acid residues, such as less than 395 consecutive amino acid residues, e.g. less than 390 consecutive amino acid residues, such as less than 385 consecutive amino acid residues, e.g. less than 380 consecutive amino acid residues, such as less than 370 consecutive amino acid residues, e.g. less than 360 consecutive amino acid residues, such as less than 350 consecutive amino acid residues, e.g. less than 345 consecutive amino acid residues, such as less than 340 consecutive amino acid residues, e.g. less than 335 consecutive amino acid residues, such as less than 330 consecutive amino acid residues, e.g. less than 325 consecutive amino acid residues, such as less than 300 consecutive amino acid residues, e.g. less than 295 consecutive amino acid residues, such as less than 290 consecutive amino acid residues, e.g. less than 285 consecutive amino acid residues, such as less than 280 consecutive amino acid residues, e.g. less than 275 consecutive amino acid residues, such as less than 270 consecutive amino acid residues, e.g. less than 265 consecutive amino acid residues, such as less than 260 consecutive amino acid residues, such as less than 255 consecutive amino acid residues, e.g. less than 250 consecutive amino acid residues, such as less than 245 consecutive amino acid residues, e.g. less than 240 consecutive amino acid residues, such as less than 235 consecutive amino acid residues, e.g. less than 230 consecutive amino acid residues, such as less than 225 consecutive amino acid residues, such as less than 220 consecutive amino acid residues, such as less than 215 consecutive amino acid residues, e.g. less than 210 consecutive amino acid residues, such as less than 205 consecutive amino acid residues, e.g. less than 200 consecutive amino acid residues, such as less than 195 consecutive amino acid residues, e.g. less than 190 consecutive amino acid residues, such as less than 185 consecutive amino acid residues, e.g. less than 180 consecutive amino acid residues, such as less than 175 consecutive amino acid residues, e.g. less than 170 consecutive amino acid residues, such as less than 165 consecutive amino acid residues, e.g. less than 160 consecutive amino acid residues, such as less than 155 consecutive amino acid residues, e.g. less than 150 consecutive amino acid residues, such as less than 145 consecutive amino acid residues, e.g. less than 140 consecutive amino acid residues, such as less than 135 consecutive amino acid residues, e.g. less than 130 consecutive amino acid residues, such as less than 125 consecutive amino acid residues, e.g. less than 120 consecutive amino acid residues, such as less than 115 consecutive amino acid residues, e.g. less than 110 consecutive amino acid residues, such as less than 105 consecutive amino acid residues, e.g. less than 100 consecutive amino acid residues, such as less than 95 consecutive amino acid residues, e.g. less than 90 consecutive amino acid residues, such as less than 85 consecutive amino acid residues, e.g. less than 80 consecutive amino acid residues, such as less than 75, e.g. less than 60 consecutive amino acid residues of SEQ ID NO: 3.

The translational product of the SLC18A2 gene according to the present invention comprises variant fragments, wherein the polypeptide variant fragment contains less than 99.5%, such as less than 98%, e.g. less than 97%, such as less than 96%, e.g. less than 95%, such as less than 94%, e.g. less than 93%, such as less than 92%, e.g. less than 91%, such as less than 90%, e.g. less than 88%, such as less than 86%, e.g. less than 84%, e.g. less than 82%, such as less than 80%, e.g. less than 75%, such as less than 70%, e.g. less than 65%, such as less than 60%, e.g. less than 55%, such as less than 50%, e.g. less than 45%, such as less than 40%, e.g. less than 35%, such as less than 30%, e.g. less than 25%, such as less than 20%, such as less than 15%, e.g. less than 10% of the amino acid residues of SEQ ID NO:3.

The following terms are used to describe the sequence relationships between two or more polynucleotides: “predetermined sequence”, “comparison window”, “sequence identity”, “percentage of sequence identity”, and “substantial identity”.

A “predetermined sequence” is a defined sequence used as a basis for a sequence comparison; a predetermined sequence may be a subset of a larger sequence, for example, as a segment of a full-length DNA, RNA or gene sequence given in a sequence listing, such as a polynucleotide sequence of SEQ ID NO:1, SEQ ID NO:2, SEQ ID NO:4, or SEQ ID NO:5, fragment or part thereof or may comprise a complete DNA, RNA or gene sequence. Generally, a predetermined sequence is at least 20 nucleotides in length, frequently at least 25 nucleotides in length, and often at least 50 nucleotides in length.

Accordingly, variants preferably have at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined nucleotide sequence SEQ ID NO:1.

In one embodiment, variants preferably have at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined nucleotide sequence SEQ ID NO:2.

In analogy, variants preferably have at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined nucleotide sequence SEQ ID NO:4.

Similarly, variants preferably have at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined nucleotide sequence SEQ ID NO:5.

The polynucleotide of the invention may encode a variant polypeptide, wherein the variant polypeptide has the polypeptide sequence of a naturally occurring polypeptide variant.

The nucleic acid sequence of the polynucleotide may differ by a single nucleotide from a nucleic acid sequence of SEQ ID NO: 1, 2, 4 or 5. However, the polynucleotide may also differ from a nucleic acid sequence a nucleic acid sequence of SEQ ID NO: 1, 2, 4 or 5 by two, 3, 4, 5, 6, 7, 8, 9, 10, or more nucleotides.

In one embodiment, the nucleic acid sequence of the polynucleotide has at least 80% sequence identity, such as preferably at least 81% sequence identity, more preferably e.g. at least 82% sequence identity, such as more preferably at least 83% sequence identity, e.g. more preferably at least 84% sequence identity, more preferably such as at least 85% sequence identity, more preferably e.g. at least 86% sequence identity, more preferably such as at least 87% sequence identity, more preferably e.g. at least 88% sequence identity, more preferably such as at least 89% sequence identity, more preferably e.g. at least 90% sequence identity, more preferably such as at least 91% sequence identity, more preferably e.g. at least 92% sequence identity, such as at least 93% sequence identity, more preferably e.g. at least 94% sequence identity, more preferably such as at least 95% sequence identity, more preferably e.g. at least 96% sequence identity, more preferably such as at least 97% sequence identity, more preferably e.g. at least 98% sequence identity, more preferably such as at least 99% sequence identity, more preferably e.g. at least 99.5% sequence identity to a nucleotide sequence a nucleic acid sequence of SEQ ID NO: 1, 2, 4 or 5.

The nucleic acid sequence of the polynucleotide may contain less than 99.5%, such as less than 98%, e.g. less than 97%, such as less than 96%, e.g. less than 95%, such as less than 94%, e.g. less than 93%, such as less than 92%, e.g. less than 91%, such as less than 90%, e.g. less than 88%, such as less than 86%, e.g. less than 84%, e.g. less than 82%, such as less than 80%, e.g. less than 75%, such as less than 70%, e.g. less than 65%, such as less than 60%, e.g. less than 55%, such as less than 50%, e.g. less than 45%, such as less than 40%, e.g. less than 35%, such as less than 30%, e.g. less than 25%, such as less than 20%, such as less than 15%, e.g. less than 10% of nucleotide sequence a nucleic acid sequence of SEQ ID NO: 1, 2, 4 or 5.

In yet another embodiment, the invention relates to nucleic acid sequences (e.g., DNA, RNA) that hybridise to nucleic acids of SLC18A2 gene, variant or part thereof. In particular, nucleic acids which hybridise to SEQ ID NO: 1, 2, 4 or 5 under high, moderate or reduced stringency conditions as described above.

In still another embodiment, the invention relates to a complement of nucleic acid of a nucleic acid sequence of SEQ ID NO: 1, 2, 4 or 5

Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a predetermined sequence of at least 20 contiguous nucleotides and wherein the portion of the polynucleotide sequence in the comparison window may comprise additions or deletions (i.e., gaps) of 20 percent or less as compared to the predetermined sequence (which does not comprise additions or deletions) for optimal alignment of the two sequences.

Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

Methods of alignment of sequences for comparison are well-known in the art. Various programs and alignment algorithms are described and present a detailed consideration of sequence alignment methods and homology calculations, such as VECTOR NTI. The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences will be.

The NCBI Basic Local Alignment Search Tool (BLAST) is available from several sources, including the National Center for Biotechnology Information (NBCI, Bethesda, Md.) and on the Internet, for use in connection with the sequence analysis programs blastp, blastn, blastx, tblastn and tblastx. It can be accessed at http://www.ncbi.nlm.nih.gov/BLAST/. A description of how to determine sequence identity using this program is available at www.ncbi.nlm.nih.gov/BLAST/blast_help.html.

Homologs of the disclosed polypeptides are typically characterised by possession of at least 94% sequence identity counted over the full length alignment with the disclosed amino acid sequence using the NCBI Basic Blast 2.0, gapped blastp with databases such as the nr or swissprot database. Alternatively, one may manually align the sequences and count the number of identical amino acids. This number divided by the total number of amino acids in your sequence multiplied by 100 results in the percent identity.

The term “promoter” refers to the regulatory region located upstream of the ATG start site, or 5′ to the structural gene. Sequence analysis of the promoter region of SLC18A2 shows that nearly 13% of the nucleotides are C or G and about 13% are CpG dinucleotides.

The term “part thereof” with reference to a nucleotide sequence refers to a portion of the stated sequence containing at least 20, or at least 30, at least 40, at least 50, at least 70, or at least 100 consecutive nucleotides. Similarly, the term “part thereof” with reference to an amino acid sequence refers to a portion of the stated sequence containing at least 7, or at least 10, at least 15, at least 20, at least 30, or at least 50 consecutive amino acids.

The term “status” with reference to an individual having prostate cancer or suspected of having prostate cancer refers to the medical or biological status of the individual in any aspect related to the state of having or potentially having prostate cancer. Such status includes whether or not the individual currently has prostate cancer, the risk the individual has of developing prostate cancer or having prostate cancer recur in the future, the stage of progression of prostate cancer within the individual, the treatment or prevention regime(s) appropriate for the individual with respect to prostate cancer, the selection of treatment agent(s) for the individual, the determination of the effectiveness of treatment regime(s) or agent(s) for the individual, and the prediction of clinical or biological outcome(s) for the individual with respect to prostate cancer and related conditions.

### Prostate Cancer

The present invention discloses a marker for prostate cancer and methods including the use of the marker gene for example assisting in diagnosing, monitoring, predicting the outcome, prognosticating the outcome of prostate cancer as described elsewhere herein. Prostate cancer is a disease wherein cancer develops in the prostate gland of the male reproductive system. Thus, prostate cancer is classified as an adenocarcinoma (glandular cancer) in which normal semen-secreting prostate gland cells mutate into cancer cells. The region of prostate gland where the adenocarcinoma is most common is the peripheral zone. Initially, small clumps of cancer cells remain confined to otherwise normal prostate glands, a condition known as carcinoma in situ or prostatic intraepithelial neoplasia (PIN). Although PIN has not been proven to be a cancer precursor, it is closely associated with cancer. Over time these cancer cells begin to multiply and spread to the surrounding prostate tissue, known as the stroma, forming a tumor. Eventually, the tumor may grow large enough to invade nearby organs such as the seminal vesicles or the rectum, or the tumor cells may develop the ability to travel in the bloodstream and lymphatic system. Prostate cancer is considered a malignant tumor because it is a mass of cells which can invade other parts of the body. This invasion of other organs is called metastasis. Prostate cancer most commonly metastasizes to the bones, lymph nodes, rectum, and bladder.

Prostate cancer is evaluated by the stage of the cancer, or how far the cancer has spread. As for the marker of the present invention, knowledge of the stage is useful in defining the prognosis of prostate cancer and for deciding on treatment regimes. The four stage TNM system (Tumor/Nodes/Metastases) is widely used for staging prostate cancer. The system makes use of information regarding the size of the tumor, the number of involved lymph nodes, and the presence of any other metastases.

The most important distinction made by any staging system is whether or not the cancer is still confined to the prostate. In the TNM system, clinical T1 and T2 cancers are found only in the prostate, while T3 and T4 cancers have spread elsewhere. Prostate cancer is staged according to the TNM system (tumor/nodes/metastases). The following terms are used to stage a prostate cancer:

### T—Primary Tumour

Tx Primary tumour cannot be assessed
T0 No evidence of primary tumour
T1 Clinically inapparent tumour neither palpable nor visible by imaging¹:
1a Tumour incidental histologic finding in 5% or less of tissue resected
1b Tumour incidental histologic finding in more than 5% of tissue resected
1c Tumour identified by needle biopsy (e.g. because of elevated PSA)
T2 Tumour confined within the prostate²:
2a Tumour involves one-half of one lobe or less
2b Tumour involves more than one-half of one lobe but not both lobes
2c Tumour involves both lobes
T3 Tumour extends through the prostatic capsule³:
3a Extracapsular extension in periprostatic tissue (unilateral or bilateral)
3b Invasion of the seminal vesicle(s)
T4 Tumour is fixed or invades adjacent structures other than the seminal vesicles:

bladder neck, external sphincter, rectum, levator muscles, or pelvic wall⁴ ¹There is no pT1 category because there is not enough tissue to determine the highest pT category.²Tumour found in one or both lobes by needle biopsy, but not palpable or visible by imaging, is classified as Tic.³Invasion into the prostatic apex but not beyond the prostate is not classified as T3, but as T2.⁴If radical prostatectomy shows that the bladder neck contains microscopic tumour, this must be classified as Tia.

### N—Regional Lymph Nodes

Nx Regional lymph nodes cannot be assessed
N0 No regional lymph node metastases
N1 Metastasis in regional lymph nodes

### M—Distant Metastasis

Mx Distant metastasis cannot be assessed
M0 No distant metastasis
M1 Distant metastasis
1a Non-regional lymph nodes

### 1b Bone(s)

1c Other site(s)

Tissue samples are examined microscopically to determine the features of any cancer identified. The so-called Gleason-score is used to help evaluating the prognosis of individuals having contracted prostate cancer. Upon examination of biopsy samples under a microscope a pathologist assigns grades to the most common tumor pattern of the sample. The two grades are added together to get a Gleason score. The Gleason grade ranges from 1 to 5, with 5 having the worst prognosis. The Gleason score ranges from 2 to 10, with 10 having the worst prognosis.

Gleason scores are associated with the following features: Grade 1—The cancerous prostate closely resembles normal prostate tissue. The glands are small, well-formed, and closely packed; Grade 2—The tissue still has well-formed glands, but they are larger and have more tissue between them; Grade 3—The tissue still has recognizable glands, but the cells are darker. At high magnification, some of these cells have left the glands and are beginning to invade the surrounding tissue; Grade 4—The tissue has few recognizable glands. Many cells are invading the surrounding tissue; Grade 5—The tissue does not have recognizable glands. There are often just sheets of cells throughout the surrounding tissue.

The present invention wherein the SLC18A2 gene, transcript or translational product thereof is disclosed as a marker of prostate cancer relates to all stages, and grades of prostate cancer in the methods disclosed herein.

### METHODS OF THE PRESENT INVENTION

The present invention of the SLC18A2 gene as a marker of prostate cancer has resulted in a number of methods useful in regard to prostate cancer. For any one of the methods it is appreciated that the methods may be used on their own with SLC18A2 used as an independent marker of prostate cancer, or in combination with other markers known. In particular the SLC18A2 marker may be used in combination with the prostate specific antigen (PSA). PSA is produced by the normal prostate, where small amounts leak into the circulation in normal circumstances. Enlarged prostates and cancerous prostates leak substantial amounts of PSA which can be measured.

One aspect of the present invention relates to a method for assisting in diagnosing and/or for diagnosing prostate cancer in an individual comprising the steps of

i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of presence or absence of prostate cancer.

According to this method for assisting in diagnosing and/or for diagnosing prostate cancer, an increased methylation status is indicative of the presence of prostate cancer. Similarly, a decreased transcriptional and/or translational expression level in the sample is indicative of the presence of prostate cancer.

Another aspect of the present invention relates to a method for assisting in prognosing and/or for prognosing the disease progression of prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
   wherein the methylation status of i) and/or the transcriptional and/or the translational expression level of ii) is indicative of the progression of prostate cancer.

According to this aspect of wherein an increased methylation status is indicative of disease progression of prostate cancer. Similarly, a decreased transcriptional and/or translational expression level in the sample is indicative disease progression of prostate cancer.

A third aspect of the present invention pertains to a method for assisting in predicting and/or for predicting the outcome of prostate cancer in an individual having contracted prostate cancer comprising the steps of

i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
wherein the methylation status of i) and/or the transcriptional and/or the translational expression level of ii) is indicative of the outcome of prostate cancer.

An increased methylation status is indicative of the disease progression of prostate cancer whereas a decreased transcriptional and/or translational expression level in the sample is indicative of the disease progression of prostate cancer.

Method for Assisting in Predicting and/or for Predicting the Recurrence Risk of Prostate Cancer

A further aspect of the present invention concerns a method for assisting in predicting and/or for predicting the recurrence risk of prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
   wherein the methylation status of i) and/or the transcriptional and/or the translational expression level of ii) is indicative of the recurrence risk of prostate cancer.

It is appreciated that the recurrence risk is the risk of cancer reccurring after intervention, for example following surgery, radical prostatectomy, radiation therapy, cryotherapy or brachytherapy. Recurrence can occur over a wide span in time, for example 2 months after intervention, such as 3 months, 4, months, 5 months, 6 months, 1 year, two years, three years, 4 years, 5, years, 6 years, 7 years, 8 years, 9 years, 10 years, 11 years, 12 years, 13 years, 14 years, 15 years, 16 years, 17 years, 18 years, 19 years, 20 years, 25 years after intervention or even longer. Thus, the methods of the present invention can be used over a wide span of time to determine whether recurrence has occurred.

Tumor recurrence is often measured by increased levels of PSA defined as PSA level greater than or equal to 0.1 ng/ml serum measured at least one month after for example surgery. Individuals having PSA levels above 0.1 ng/ml serum measured less than 1 month after surgery, are not considered to suffer from reoccurrence but rather an indication that the primary tumor was not removed, for example in the event of advanced cancer at the time of surgery. In rare cases metastases are detected without an increase in PSA level. The present invention provides a marker which can be used independently of other markers, or in combination with other markers and techniques, for example in combination with PSA measurements.

According to this aspect of the present invention an increased methylation status is indicative of a risk of recurrence of prostate cancer at any of the indicated times after intervention. Similarly, a decrease in the transcriptional and/or translational expression level in the sample is indicative of risk of recurrence of prostate cancer.

In one embodiment the risk of recurrence is determined following radical prostatectomy.

According to the examples of present invention overall survival is defined as survival, whereas recurrence-free survival corresponds to cancer-specific survival which is survival from prostate cancer where the individual may die from other causes than prostate cancer. The recurrence is recurrence-free survival and overall survival

A further aspect of the present invention pertains to a method for assisting in monitoring and/or for monitoring the effect of treatment on prostate cancer progression in an individual having contracted prostate cancer, comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
   wherein the methylation status of i) and/or the transcriptional and/or the translational expression level of ii) is indicative of progression of said prostate cancer.

An increased methylation status is indicative of a progression of said prostate cancer.

Similarly, a decrease in the transcriptional and/or translational expression level of the SLC18A2 gene in the sample is indicative a progression of said prostate cancer.

Assisting in Monitoring and/or for Monitoring the Progression of Prostate Cancer from a Silent to an Aggressive Prostate Cancer

Individuals suspected of having contracted, or suspected to be at risk of contracting prostate cancer, or having been diagnosed with prostate cancer may be enrolled in an active surveillance programme. Active surveillance refers to observation and regular monitoring of outbreak without invasive treatment. Thus, active surveillance is often used when an early stage, slow-growing prostate cancer is suspected. For younger men (>10 years life expectancy), a programme of active surveillance may not mean avoiding treatment altogether, but may allow a delay of a few years or more, during which time the quality of life impact of active treatment can be avoided carefully selected men will not miss a window for cure with this approach. Careful selection of individuals for active surveillance ensures that the selected individuals will not be less prone to cure than other individuals.

Programmes for ‘watchful waiting’ may also be suggested when the risks of surgery, radiation therapy, or hormonal therapy outweigh the possible benefits. Other treatments can be started if symptoms develop, or if there are signs that the cancer growth is accelerating (e.g., rapidly rising PSA, increase in Gleason score on repeat biopsy, etc.).

The SLC18A2 marker of the present invention can be used for monitoring prostate cancer progression, as an independent marker, but preferably with other markers such as the PSA marker, and characterisation of stage and grade of the prostate cancer in said individual.

The present invention thus in one aspect relates to a method for assisting in monitoring and/or for monitoring the progression of prostate cancer from a silent to an aggressive prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
   wherein the methylation status of i) and/or the transcriptional and/or the translational expression level of ii) is indicative of the progression of prostate cancer from a silent/indolent to an aggressive prostate cancer.

It is appreciated that an aggressive prostate cancer may require treatment in order for the disease not to progress further, in an attempt to contain the disease.

An increased methylation status is indicative of a progression of the prostate cancer and a decrease in the transcriptional and/or translational expression level of the SLC18A2 gene in the sample is indicative a progression of said prostate cancer.

It is appreciated that a silent/indolent prostate cancer is a slow-growing and slow-progressing organ-confined prostate cancer with no or only minor clinical symptoms, such as obstructive voiding symptoms. Individuals with silent/indolent prostate cancer are more likely to die with prostate cancer than from prostate cancer. The majority of low grade prostate cancers (Gleason score 2-4) and/or low stage (T1a/b/c-T2a) prostate cancers, typically also having low serum PSA levels (<10 ng/mL) are in the present context comprised within the term silent/indolent prostate cancer.

Silent prostate cancer also include prostate cancers with low risk of recurrence following a given treatment, e.g. radical prostatectomy, as well as prostate cancers that do not cause considerable morbidity or mortality to an individual even if left untreated (active surveillance).

An aggressive prostate cancer according to the present invention is a prostate cancer which has progressed or will progress relatively fast (i.e. within the remaining life expectancy of a given patient) to non-organ-confined prostate cancer (locally advanced prostate cancer and/or dissiminated prostate cancer with distant metastasis and/or castration-resistant prostate cancer) and which will cause severe morbidity and high risk of mortality to the patient. In contrast to patients with indolent/silent prostate cancer, patients with aggressive prostate cancer are likely to die from prostate cancer rather than with prostate cancer. The majority of high grade prostate cancers (Gleason score 8-10) and/or high stage (T3-T4), typically also having high serum PSA levels (>10 ng/mL) are in the present context comprised within the term aggressive prostate cancer.

The term aggressive prostate cancer also includes prostate cancers with high risk of recurrence after treatment such as radical prostatectomy.

Method for Assisting in Determining and/or Determining the Treatment Regime of an Individual Having Contracted Prostate Cancer Comprising the Steps of

A further aspect of the present invention relates to a method for assisting in determining and/or determining the treatment regime of an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample
   wherein the methylation status of i) and/or the transcriptional and/or the translational expression level of ii) is indicative of the treatment regime to be offered to the individual having contracted prostate cancer.

Still another aspect of the present invention relates to a method for determining or for assisting in determining the status of an individual having or suspected of having prostate cancer. The method includes the steps of:
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
   wherein the methylation status of i) and/or the transcriptional and/or the translational expression level of ii) is indicative of the status of said individual. Determining the status of the individual can include determination of any status of the individual related to prostate cancer, including but not limited to diagnosing, prognosing the disease progression, predicting the outcome, predicting the recurrence risk, monitoring the effect of treatment, monitoring the progression, and determining a treatment regime.

According to this aspect an increased methylation status is indicative of the presence and/or the progression of said prostate cancer. Analogously, a decreased transcriptional and/or translational expression level in the sample is indicative of the presence and/or the progression of said prostate cancer.

For all of the listed methods the methods may comprise a a further step of administrating a methylation modulating agent, where the methylation status, the transcriptional and/or translational expression level is indicative of the presence and/or the progression of said prostate cancer.

Additionally, for all the methods of the present inventions, the methods may further comprise a step of measuring the level of prostate specific antigen (PSA) in an individual.

In one embodiment the individual has a normal PSA level of less than 4 ng per ml serum.

In another embodiment the individual has a PSA level higher than a normal PSA level, corresponding to a PSA level above 4 ng per ml serum.

Digital rectal examination (DRE) can be used for diagnosing the presence or absence of prostate cancer in an individual, DRE can therefore be used in combination with the method for diagnosing prostate cancer employing SLC18A2 as a marker. Most CaPs are located in the peripheral zone of the prostate and may be detected by DRE when the volume is about 0.2 mL or larger. The risk of a positive DRE turning out to be cancer is heavily dependent on the PSA value. When PSA is measured at a concentration of 0-1 ng/ml, then the positive predictive value for cancer is 2.8 to 5%. With PSA concentrations in the range of 1-2.5 ng/ml serum, the positive predictive value for cancer is 10.5 to 14%. PSA concentrations in the range of 2.5 to 4 ng/ml serum, the positive predictive value for cancer is 22 to 30%. For PSA concentrations in the range of 4-10 ng/ml serum, the positive predictive value for cancer is 41%, whereas PSA concentrations above 10 ng/ml results in a positive predictive value of 69%.

### Samples

The sample that is used in the methods of the present invention may be in a form suitable to allow analysis by the skilled artisan. The samples according to the present invention may be selected from a tissue sample, or from body fluids such as blood, plasma, serum, semen, or urine. In a preferred embodiment of the present invention the sample is a urine sample, blood sample, and/or a tissue sample. In case of urine samples a preferred urine sample is a urine sample where the prostate gland has been massaged prior to the sampling in order to transfer as many cells of prostate origin to the urine.

In one particular embodiment of the present invention, the sample is a tissue sample, such as a biopsy of the tissue, or a superficial sample scraped from the tissue. In another embodiment the sample may be prepared by forming a suspension of cells made from the tissue. The sample may, however, also be an extract obtained from the tissue or obtained from a cell suspension made from the tissue. It is appreciated that that for the various methods of the present invention different tissue samples are preferred or available. For assisting in the diagnosis/diagnosing prostate cancer according to the present invention the tissue sample is typically a biopsy of the prostate gland. However, the sample can also be a urine, sample or blood sample. In a particular embodiment the sample to be used for diagnosing is a urine sample. The urine sample may be obtained following the massage of the prostate gland, whereby prostate cells are released into the urine. In a preferred embodiment the sample used in the diagnosis of prostate cancer is a biopsy of the prostate gland,

For assisting in the prognosis and/or for the prognosis of the disease progression of prostate cancer; for assisting in the prediction and/or the prediction of the progression of prostate cancer; for assisting in predicting and/or for predicting the recurrence risk of prostate cancer; for assisting in monitoring and/or for monitoring the effect of treatment on prostate cancer progression; for assisting in monitoring and/or for monitoring the progression of prostate cancer from a silent to an aggressive prostate cancer, and/or for assisting in determining and/or determining the treatment regime it is preferred that the sample is from a biopsy of the prostate tissue, from a resected prostate cancer tumour, or from a resected prostate following radical prostatectomy.

Working with tumor material requires biopsies or body fluids suspected to comprise relevant cells. Working with RNA requires freshly frozen or immediately processed biopsies, or chemical pretreatment of the biopsy. Apart from the cancer tissue, biopsies do inevitably contain many different cell types, such as cells present in the blood, connective and muscle tissue, endothelium etc. In the case of DNA studies, microdissection or laser capture are methods of choice, however the time-dependent degradation of RNA makes it difficult to perform manipulation of the tissue for more than a few minutes. The sample may be fresh or frozen, or treated with chemicals.

### Control Sample

A control sample is any corresponding sample (tissue sample, blood, plasma, serum, semen, or urine) that is non-malignant, i.e. taken from an individual without prostate cancer, for example a non-malignant prostate biopsy of a healthy individual.

The control sample should be used as a standard level of the methylation status to be used in evaluation whether the methylation status of a sample to be tested is increased or decreased. For determination of the methylation status of the SLC18A2 gene, the control sample is a DNA fragment with a sequence corresponding to that of the promoter region or part thereof of the SLC18A2 gene, wherein the methylation status is known, for example a non-methylated DNA fragment. The DNA fragment is in one embodiment be of synthetic origin, however, in another embodiment the DNA fragment is derived from DNA extracted from DNA vector constructs, comprising said promoter region or part thereof.

By the term control sample is also meant a standard transcriptional and/or translational expression level of the SLC18A2 gene. Such a standard level is determined in samples from non-diseased prostate glands of a statistically significant number of healthy individuals such as at least 2, 3, 4, 5, 6, 7, 8, 9, 10, 15, 20, 30, 40, 50 or more healthy individuals. Such a standard transcriptional and/or translational expression level from non-diseased prostate glands of a statistically significant number of healthy individuals forms the cut off value. If the transcriptional and/or translational expression level from a test sample is below said cut off value the expression level is decreased and thus indicative of prostate cancer according to the methods of the present invention.

### Determining the Methylation Status

The methods of the present invention comprises a step of determining the methylation status of a SLC18A2 gene (SEQ ID NO:1), or nucleotide sequence having at least 90% sequence identity with SEQ ID NO.: NO:1, or part thereof in a sample from the individual.

In one embodiment the methylation status is determined in the region of the SLC18A2 gene ranging from 5000 base pairs upstream of the ATG codon to 5000 base pairs downstream of the ATG codon. In another embodiment the methylation status of the SLC18A2 gene or part thereof is determined for example of the region corresponding to SEQ ID NO.: 4. In yet another embodiment the methylation status of the SLC18A2 gene or part thereof is determined for example of the region corresponding to SEQ ID NO.: 5.

In one preferred embodiment the methylation status is determined in the region of the SLC18A2 gene corresponding to SEQ ID NO.: 4 ranging from 795 base pairs to 383 base pairs upstream of the ATG codon. In one embodiment the methylation status is determined in position 1 base pairs to 100 base pairs of SEQ ID NO: 4. In another embodiment the methylation status is determined in position 100 base pairs to 200 base pairs of SEQ ID NO: 4. In another embodiment the methylation status is determined in position 200 base pairs to 300 base pairs of SEQ ID NO: 4. In another embodiment the methylation status is determined in position 300 base pairs to 412 base pairs of SEQ ID NO: 4. In one embodiment the methylation status is determined in position 1 basepair to 50 base pairs of SEQ ID NO: 4. In another embodiment the methylation status is determined in position 50 basepair to 100 base pairs of SEQ ID NO: 4. In another embodiment the methylation status is determined in position 100 base pairs to 150 base pairs of SEQ ID NO: 4. In another embodiment the methylation status is determined in position 150 base pairs to 200 base pairs of SEQ ID NO: 4. In another embodiment the methylation status is determined in position 200 base pairs to 250 base pairs of SEQ ID NO: 4. In another embodiment the methylation status is determined in position 250 base pairs to 300 base pairs of SEQ ID NO: 4. In another embodiment the methylation status is determined in position 300 base pairs to 350 base pairs of SEQ ID NO: 4. In another embodiment the methylation status is determined in position 350 base pairs to 412 base pairs of SEQ ID NO: 4.

In one preferred embodiment the methylation status is determined in the region of the SLC18A2 gene corresponding 980 to base pairs to 795 base pairs upstream of the ATG codon. In another preferred embodiment the methylation status is determined at the CpG dinucleotide at position −969 upstream of the ATG codon.

It is appreciated that the methylation status of at least one CpG dinucleotide is determined, such as two, three, four, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or more that 100 CpG dinucleotides.

### DNA Extraction Prior to Determining Methylation Status

Prior to the determination of the methylation status DNA is extracted from the sample. For those embodiments where whole cells, or other tissue samples are being analyzed, it will typically be necessary to extract the nucleic acids from the cells or viruses, prior to continuing with the various sample preparation operations. Accordingly, following sample collection, nucleic acids may be liberated from the collected cells, viral coat etc. into a crude extract followed by additional treatments to prepare the sample for subsequent operations, such as denaturation of contaminating (DNA binding) proteins, purification, filtration and desalting.

Liberation of nucleic acids from the sample cells, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins.

Alternatively, physical methods may be used to extract the nucleic acids and denature DNA binding proteins, such as physical protrusions within microchannels or sharp edged particles piercing cell membranes and extract their contents. Combinations of such structures with piezoelectric elements for agitation can provide suitable shear forces for lysis.

More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. Subjecting cells to ultrasonic agitation, or forcing cells through microgeometry apertures, thereby subjecting the cells to high shear stress resulting in rupture are also possible extraction methods.

Following extraction, it will often be desirable to separate the nucleic acids from other elements of the crude extract, e.g. denatured proteins, cell membrane particles and salts. Removal of particulate matter is generally accomplished by filtration or flocculation. Further, where chemical denaturing methods are used, it may be desirable to desalt the sample prior to proceeding to the next step. Desalting of the sample and isolation of the nucleic acid may generally be carried out in a single step, e.g. by binding the nucleic acids to a solid phase and washing away the contaminating salts, or performing gel filtration chromatography on the sample passing salts through dialysis membranes. Suitable solid supports for nucleic acid binding include e.g. diatomaceous earth or silica (i.e., glass wool). Suitable gel exclusion media also well known in the art may be readily incorporated into the devices of the present invention and is commercially available from, e.g., Pharmacia and Sigma Chemical.

Alternatively, desalting methods may generally take advantage of the high electrophoretic mobility and negativity of DNA compared to other elements. Electrophoretic methods may also be utilized in the purification of nucleic acids from other cell contaminants and debris. Upon application of an appropriate electric field, the nucleic acids present in the sample will migrate toward the positive electrode and become trapped on the capture membrane. Sample impurities remaining free of the membrane are then washed away by applying an appropriate fluid flow. Upon reversal of the voltage, the nucleic acids are released from the membrane in a substantially purer form. Further, coarse filters may also be overlaid on the barriers to avoid any fouling of the barriers by particulate matter, proteins or nucleic acids, thereby permitting repeated use.

In a similar aspect, the high electrophoretic mobility of nucleic acids with their negative charges, may be utilized to separate nucleic acids from contaminants by utilizing a short column of a gel or other appropriate matrices or gels which will slow or retard the flow of other contaminants while allowing the faster nucleic acids to pass.

This invention provides nucleic acid affinity matrices that bear a large number of different nucleic acid affinity ligands allowing the simultaneous selection and removal of a large number of preselected nucleic acids from the sample. Methods of producing such affinity matrices are also provided. In general the methods involve the steps of a) providing a nucleic acid amplification template array comprising a surface to which are attached at least 50 oligonucleotides having different nucleic acid sequences, and wherein each different oligonucleotide is localized in a predetermined region of said surface, the density of said oligonucleotides is greater than about 60 different oligonucleotides per 1 cm.sup.2, and all of said different oligonucleotides have an identical terminal 3′ nucleic acid sequence and an identical terminal 5′ nucleic acid sequence. b) amplifying said multiplicity of oligonucleotides to provide a pool of amplified nucleic acids; and c) attaching the pool of nucleic acids to a solid support.

For example, nucleic acid affinity chromatography is based on the tendency of complementary, single-stranded nucleic acids to form a double-stranded or duplex structure through complementary base pairing. A nucleic acid (either DNA or RNA) can easily be attached to a solid substrate (matrix) where it acts as an immobilized ligand that interacts with and forms duplexes with complementary nucleic acids present in a solution contacted to the immobilized ligand. Unbound components can be washed away from the bound complex to either provide a solution lacking the target molecules bound to the affinity column, or to provide the isolated target molecules themselves. The nucleic acids captured in a hybrid duplex can be separated and released from the affinity matrix by denaturation either through heat, adjustment of salt concentration, or the use of a destabilizing agent such as formamide, TWEEN™-20 denaturing agent, or sodium dodecyl sulfate (SDS).

Affinity columns (matrices) are typically used either to isolate a single nucleic acid typically by providing a single species of affinity ligand. Alternatively, affinity columns bearing a single affinity ligand (e.g. oligo dt columns) have been used to isolate a multiplicity of nucleic acids where the nucleic acids all share a common sequence (e.g. a polyA).

The type of affinity matrix used depends on the purpose of the analysis. For example, where it is desired to analyze mRNA expression levels of particular genes in a complex nucleic acid sample (e.g., total mRNA) it is often desirable to eliminate nucleic acids produced by genes that are constitutively overexpressed and thereby tend to mask gene products expressed at characteristically lower levels. Thus, in one embodiment, the affinity matrix can be used to remove a number of preselected gene products (e.g., actin, GAPDH, etc.). This is accomplished by providing an affinity matrix bearing nucleic acid affinity ligands complementary to the gene products (e.g., mRNAs or nucleic acids derived therefrom) or to subsequences thereof. Hybridization of the nucleic acid sample to the affinity matrix will result in duplex formation between the affinity ligands and their target nucleic acids. Upon elution of the sample from the affinity matrix, the matrix will retain the duplexes nucleic acids leaving a sample depleted of the overexpressed target nucleic acids.

The affinity matrix can also be used to identify unknown mRNAs or cDNAs in a sample. Where the affinity matrix contains nucleic acids complementary to every known gene (e.g., in a cDNA library, DNA reverse transcribed from an mRNA, mRNA used directly or amplified, or polymerized from a DNA template) in a sample, capture of the known nucleic acids by the affinity matrix leaves a sample enriched for those nucleic acid sequences that are unknown. In effect, the affinity matrix is used to perform a subtractive hybridization to isolate unknown nucleic acid sequences. The remaining “unknown” sequences can then be purified and sequenced according to standard methods.

The affinity matrix can also be used to capture (isolate) and thereby purify unknown nucleic acid sequences. For example, an affinity matrix can be prepared that contains nucleic acid (affinity ligands) that are complementary to sequences not previously identified, or not previously known to be expressed in a particular nucleic acid sample. The sample is then hybridized to the affinity matrix and those sequences that are retained on the affinity matrix are “unknown” nucleic acids. The retained nucleic acids can be eluted from the matrix (e.g. at increased temperature, increased destabilizing agent concentration, or decreased salt) and the nucleic acids can then be sequenced according to standard methods.

Similarly, the affinity matrix can be used to efficiently capture (isolate) a number of known nucleic acid sequences. Again, the matrix is prepared bearing nucleic acids complementary to those nucleic acids it is desired to isolate. The sample is contacted to the matrix under conditions where the complementary nucleic acid sequences hybridize to the affinity ligands in the matrix. The non-hybridized material is washed off the matrix leaving the desired sequences bound. The hybrid duplexes are then denatured providing a pool of the isolated nucleic acids. The different nucleic acids in the pool can be subsequently separated according to standard methods (e.g. gel electrophoresis).

As indicated above the affinity matrices can be used to selectively remove nucleic acids from virtually any sample containing nucleic acids (e.g. in a cDNA library, DNA reverse transcribed from an mRNA, mRNA used directly or amplified, or polymerized from a DNA template, and so forth). The nucleic acids adhering to the column can be removed by washing with a low salt concentration buffer, a buffer containing a destabilizing agent such as formamide, or by elevating the column temperature.

In one particularly preferred embodiment, the affinity matrix can be used in a method to enrich a sample for unknown RNA sequences (e.g. expressed sequence tags (ESTs)). The method involves first providing an affinity matrix bearing a library of oligonucleotide probes specific to known RNA (e.g., EST) sequences. Then, RNA from undifferentiated and/or unactivated cells and RNA from differentiated or activated or pathological (e.g., transformed) or otherwise having a different metabolic state are separately hybridized against the affinity matrices to provide two pools of RNAs lacking the known RNA sequences.

In a preferred embodiment, the affinity matrix is packed into a columnar casing. The sample is then applied to the affinity matrix (e.g. injected onto a column or applied to a column by a pump such as a sampling pump driven by an autosampler). The affinity matrix (e.g. affinity column) bearing the sample is subjected to conditions under which the nucleic acid probes comprising the affinity matrix hybridize specifically with complementary target nucleic acids. Such conditions are accomplished by maintaining appropriate pH, salt and temperature conditions to facilitate hybridization as discussed above.

### Detection of Methylation Status

A number of current methodologies for methylation studies exist. Sequencing of bisulphite-treated DNA is the gold standard for methylation studies as it reveals directly the status of each CpG dinucleotide. Bisulphate-based methylation genomic sequencing is capable of detecting every methylated cytosine on both strands of any target sequence, using DNA isolated from fewer than 100 cells. In this method, sodium bisulphite is used to convert cytosine residues to uracil residues in single-stranded DNA, under conditions whereby 5-methylcytosine remains non-reactive. The converted DNA is amplified with specific primers and sequenced. All the cytosine residues remaining in the sequence represent previously methylated cytosines in the genome. This method utilizes defined procedures that maximize the efficiency of denaturation, bisulphite conversion and amplification, to permit methylation mapping of single genes from small amounts of genomic DNA, readily available from germ cells and early developmental stages.

Methylation specific PCR (MSP) is one of the most widely used assay for the sensitive detection of methylation. U.S. Pat. No. 5,786,146 discloses a method of methylation specific PCR (MSP) for identifying DNA methylation patterns in a CpG containing nucleic acid. The method uses agents to modify unmethylated cytosine in the nucleic acid. Prior to amplification, the DNA is treated with sodium bisulphite to convert all unmethylated cytosines to uracils. The bisulphite reaction effectively converts methylation information into sequence difference. CpG specific oligonucleotide primers are used to distinguish between modified methylated and unmethylated nucleic acid. The identification of the methylated nucleic acid is based on the presence or absence of amplification product resulting from the amplification and distinguishing modified methylated and non-methylated nucleic acids. The generated PCR product can for example be visualized on a gel.

A critical parameter for the specificity of methylation-specific PCR is determined by primer design. Since modification of DNA by bisulfite destroys strand complementarity, either strand can serve as the template for subsequent PCR amplification, and the methylation pattern of each strand can then be determined. It will be appreciated, though, that amplifying a single strand (e.g., sense) is preferable in practice. Primers are designed to amplify a region that is 80-250 by in length, which incorporates a sufficient number of cytosines in the original strand to assure that unmodified DNA does not serve as a template for the primers. In addition, the number and position of cytosines within the CpG dinucleotide determines the specificity of the primers for methylated and unmethylated templates. Typically, 1-3 CpG sites are included in each primer and concentrated in the 3′ region of each primer. This provides optimal specificity and minimizes false positives due to mispriming. To facilitate simultaneous analysis of each of the primers of a given gene in the same thermocycler, the length of the primers is adjusted to give nearly equal melting/annealing temperatures.

Real-time fluorescent MSP (MethyLight) is based on real time PCR employing fluorescent probes in conjunction with MSP and allows for a homogeneous reaction which is of higher throughput. If the probe does not contain CpGs, the reaction is essentially a quantitative version of MSP. However, the fluorescent probe is typically designed to anneal to a site containing one or more CpGs, and this third oligonucleotide increases the specificity of the assay for completely methylated target strands. Because the detection of the amplification occurs in real time, there is no need for a secondary electrophoresis step. Since there is no post PCR manipulation of the sample, the risk of contamination is reduced. The MethyLight probe can be of any format including but not limited to a Taqman probe or a LightCycler hybridization probe pair and if multiple reporter dyes are used, several probes can be performed simultaneously [Eads (1999) Cancer Res. 59:2302-2306; Eads (2000) Nucleic Acids Res. 28:E32; Lo (1999) Cancer Res. 59:3899-390]. Real-time fluorescent MSP is the preferred method for determining the methylation status according to the present invention.

Methods such as a PCR-based high-resolution melting analysis assay may, however, also be used to determine the methylation status. Methylation-sensitive high-resolution melting (MS-HRM) analysis is another PCR-based technology which can be used for determination of SLC18A2 status and for highly specific and highly sensitive detection of methylated SLC18A2. This method takes advantage of the fact that methylated DNA and unmethylated DNA acquire different sequences after bisulphite treatment, which results in PCR products with markedly different melting profiles/temperature. PCR is used to amplify both methylated and unmethylated sequences in the same reaction, and this method can be optimised to detect methylation levels as low as 0.1%. MS-HRM also allows estimation of methylation levels by comparison of melting profiles for a test sample to the melting profiles of PCR products derived from standards with known ratios of methylated:unmethylated alleles. MS-HRM analysis protocols are simple and the method is characterized by high reproducibilty (Wojdacz T K, Dobrovic A, Hansen L L. 2008, Nat. Protoc. 2008; 3(12):1903-8).

SLC18A2 methylation status can also be measured quantitatively by pyrosequencing of bisulfite converted SLC18A2 DNA following PCR amplification. Pyrosequencing is a sequencing-by-synthesis method that relies on the sequential addition and incorporation of nucleotides in a primer-directed polymerase extension. Only one of the four nucleotides is present at any time in the reaction vessel, and only if the added nucleotide is complementary to the template DNA will it be incorporated by a DNA polymerase. This event is monitored in real time and hence can be used to quantitate the ratio between methylated and unmethylated CpG dinucleotides. The pyrosequencing technology makes use of the release of PPᵢ molecules during the iterative incorporation of unmodified nucleotides that are quantitatively converted into a bioluminometric signal (Tost, J. & Gut, I. G. DNA methylation analysis by pyrosequencing, Nature Protocols 2, -2265-2275 (2007)

SLC18A2 methylation status may also be determined by targeted resequencing of bisulfite converted DNA (or PCR amplicons hereof) using massively parallel sequencing technologies (aka next-generation sequencing) and possibly future sequencing platforms able to sequence individual DNA molecules. This would allow digital quantification of the ratio between methylated and unmethylated CpG dinucleotide(s). Next-generation sequencing platforms are characterized by the ability to process millions of sequence reads in parallel rather than 96 at a time, as typically seen for capillary-based sequencing. The workflow to produce next-generation sequence-ready libraries is straightforward; DNA fragments are prepared for sequencing by ligating specific adaptor oligos to both ends of each DNA fragment. Importantly, relatively little input DNA (a few micrograms at most) is needed to produce a library. Currently available platforms for next-generation sequencing produce shorter read lengths (35-250 bp, depending on the platform), but longer reads may be possible in the future. (Mardis, E. R. The impact of next-generation sequencing technology on genetics, Trends Genet. 2008 March; 24(3):133-41.

For rapid assessment of CpG methylation density of a DNA region the quantitative methylation density assay may be used as previously described by Galm et al. (2002) Genome Res. 12, 153-7. After bisulfite modification of genomic DNA, the region of interest is PCR amplified with nested primers. PCR products are purified and DNA amount is determined. A predetermined amount of DNA is incubated with .sup.3H-SAM (TRK581 Bioscience, Amersham) and Sssl methyltransferase for methylation quantification. Once reactions are terminated products are purified from the in-vitro methylation mixture. 20% of the eluant volume is counted in .sup.3H counter. Normalizing radioactivity DNA of each sample is measured again and the count is normalized to the DNA amount.

Restriction analysis of bisulphite modified DNA is a yet another quantitative technique which can be used to determine DNA methylation levels at specific gene loci in small amounts of genomic DNA. Restriction enzyme digestion is used to reveal methylation-dependent sequence differences in PCR products of sodium bisulfite-treated DNA. Methylation levels in original DNA sample are represented by the relative amounts of digested and undigested PCR product in a linearly quantitative fashion across a wide spectrum of DNA methylation levels. This technique can be reliably applied to DNA obtained from microdissected paraffin-embedded tissue samples.

In another embodiment, differential methylation hybridization (DMH) may be used to determine the methylation status of the promoter region of the SLC18A2 gene. DHM integrates a high-density, microarray-based screening strategy to detect the presence or absence of methylated CpG dinucleotide genomic fragments. Array-based techniques are used when a number (e.g., >3) of methylation sites in a single region are to be analyzed. First, CpG dinucleotide nucleic acid fragments from a genomic library are generated, amplified and affixed on a solid support to create a CpG dinucleotide rich screening array. Amplicons are generated by digesting DNA from a sample with restriction endonucleases which digest the DNA into fragments but leaves the methylated CpG islands intact. These amplicons are used to probe the CpG dinucleotide rich fragments affixed on the screening array to identify methylation patterns in the CpG dinucleotide rich regions of the DNA sample. Unlike other methylation analysis methods such as Southern hybridization, bisulfite DNA sequencing and methylation-specific PCR which are restricted to analyzing one gene at a time, DMH utilizes numerous CpG dinucleotide rich genomic fragments specifically designed to allow simultaneous analysis of multiple of methylation-associated genes in the genome (for further details see U.S. Pat. No. 6,605,432).

In yet another embodiment, immunoprecipitation of methylated sequences can be used to isolate sequence-specific methylated DNA fragments. Briefly, genomic DNA is sonicated to yield fragments of 200-300 bp. The DNA is then denatured, precleaned with a protein A Fast FlowSepharose) and further incubated with a 5-methylcytidine monoclonal antibody. The complex may be purified using protein A Sepharose and subsequently washed. The immunoprecipitated samples are then analyzed using specific PCR primers.

In the present application the level of methylation may be expressed as percentage of methylation, wherein the percentage is the percentage of cells exhibiting methylation of the promoter as described herein, and/or the percentage of possible methylation sites on the promoter being methylated in a given cell.

Thus, percentage of methylation may either be provided as a percentage of afflicted cells or a percentage or methylation in the cells, or an average percentage in a group of cells, or both percentages may be provided. For both values it is emphasized that the more cells and/or the more methylation sites that are methylated the worse the diagnosis and prognosis, as described above.

The methylation status of SLC18A2 relates to the indications of prostate cancer of the sample tested, such as diagnosing, predicting, prognosing, monitoring prostate cancer in an individual. The methylation status SLC18A2 gene may be detected in either a tissue sample as such, or in a body fluid sample, such as blood, serum, plasma, semen and/or urine of the individual.

In the present invention the methylation level has been determined to be 52%-88% in adenocarcinoma cell lines, 17% in benign prostate cell lines, and 0% in non.prostate cell lines. For clinical malignant samples the methylation status ranged from 17% to 88%. When in the present invention the methylation status of the SLC18A2 gene in a sample is increased by 1% or for example 2%, 3%, 4%, 5%, 6, 7%, 8%, 9%, 10%, 15%, 20%, 25%, 30%, 35%, 40%, 41%, 42%, 43%, 44%, 45%, 46%, 47%, 48%, 49%, or at least 50% and even more, relative to previous measurements on the same individual, or to measurements on non-diseased tissue from the same individual, or relative to a standard level, the methylation status of the SLC18A2 gene is indicative of the presence of prostate cancer.

In a clinical setting, the observation of 1% methylation, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95% methylation or more is indicative of the presence of prostate cancer, when the sample is a tissue sample.

In one embodiment the methylation, indicative of prostate cancer, may be in the range from 1% to at least 50%, such as for example 1% to at least 50%, such as 1% to at least 40%, such as for example 1% to at least 30%, such as 1% to at least 20%, such as 1% to at least 10%, such as for example 1% to at least 5%. In another embodiment the methylation, indicative of prostate cancer, may be in the range from 50% to at least 95%, such as for example 50% to at least 85%, such as 50% to at least 75%, such as for example 50% to at least 65%, such as 50% to at least 65%, such as 50% to at least 60%. In another embodiment the methylation, indicative of prostate cancer, may be in the range from 25% to at least 75%, such as for example 30% to at least 70%, such as 35% to at least 65%, such as for example 40% to at least 60%, such as 45% to at least 55%. In one preferred embodiment the methylation level is in the range from 17% to at least 88%

In one preferred embodiment the methylation level in adenocarcinoma cell lines, indicative of prostate cancer, is in the range of 52% to at least 88%. In another preferred embodiment the methylation level in benign prostate cell lines is in the range of 15% to at least 20%. In one preferred embodiment the methylation level in benign prostate cell lines is 17%.

In case the sample is a urine sample, all methylation values above 0% methylation are considered to be indicative of prostate cancer, such as 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95% methylation or more.

In one embodiment the methylation level of DNA from urine samples, indicative of prostate cancer, may be in the range from 1% to at least 50%, such as for example 1% to at least 50%, such as 1% to at least 40%, such as for example 1% to at least 30%, such as 1% to at least 20%, such as 1% to at least 10%, such as for example 1% to at least 5%. In another embodiment the methylation, indicative of prostate cancer, may be in the range from 50% to at least 95%, such as for example 50% to at least 85%, such as 50% to at least 75%, such as for example 50% to at least 65%, such as 50% to at least 65%, such as 50% to at least 60%. In another embodiment the methylation, indicative of prostate cancer, may be in the range from 25% to at least 75%, such as for example 30% to at least 70%, such as 35% to at least 65%, such as for example 40% to at least 60%, such as 45% to at least 55%.

### Determining Expression Levels

### Extraction of RNA

RNA or protein can be isolated and assayed from a test sample using any techniques known in the art. They can for example be isolated from a fresh or frozen biopsy, from formalin-fixed tissue, from body fluids, such as blood, plasma, serum, urine or semen.

Methods of isolating total mRNA are well known to those of skill in the art. In one embodiment, the total nucleic acid is isolated from a given sample using, for example, an acid guanidinium-phenol-chloroform extraction method and polyA.sup. and mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

The sample may be from tissue and/or body fluids, as defined elsewhere herein. Before analyzing the sample, e.g., on an oligonucleotide array, it will often be desirable to perform one or more sample preparation operations upon the sample. Typically, these sample preparation operations will include such manipulations as extraction of intracellular material, e.g., nucleic acids from whole cell samples, viruses, amplification of nucleic acids, fragmentation, transcription, labeling and/or extension reactions. One or more of these various operations may be readily incorporated into the device of the present invention.

For a number of applications, it may be desirable to extract and separate messenger RNA from cells, cellular debris, and other contaminants. As such, the device of the present invention may, in some cases, include a mRNA purification chamber or channel. In general, such purification takes advantage of the poly-A tails on mRNA. In particular and as noted above, poly-T oligonucleotides may be immobilized within a chamber or channel of the device to serve as affinity ligands for mRNA. Poly-T oligonucleotides may be immobilized upon a solid support incorporated within the chamber or channel, or alternatively, may be immobilized upon the surface(s) of the chamber or channel itself. Immobilization of oligonucleotides on the surface of the chambers or channels may be carried out by methods described herein including, e.g., oxidation and silanation of the surface followed by standard DMT synthesis of the oligonucleotides.

In operation, the lysed sample is introduced to a high salt solution to increase the ionic strength for hybridization, whereupon the mRNA will hybridize to the immobilized poly-T. The mRNA bound to the immobilized poly-T oligonucleotides is then washed free in a low ionic strength buffer. The poly-T oligonucleotides may be immobiliized upon poroussurfaces, e.g., porous silicon, zeolites silica xerogels, scintered particles, or other solid supports.

Following sample preparation, the sample can be subjected to one or more different analysis operations. A variety of analysis operations may generally be performed, including size based analysis using, e.g., microcapillary electrophoresis, and/or sequence based analysis using, e.g., hybridization to an oligonucleotide array.

In the latter case, the nucleic acid sample may be probed using an array of oligonucleotide probes. Oligonucleotide arrays generally include a substrate having a large number of positionally distinct oligonucleotide probes attached to the substrate. These arrays may be produced using mechanical or light directed synthesis methods which incorporate a combination of photolithographic methods and solid phase oligonucleotide synthesis methods.

The basic strategy for light directed synthesis of oligonucleotide arrays is as follows. The surface of a solid support, modified with photosensitive protecting groups is illuminated through a photolithographic mask, yielding reactive hydroxyl groups in the illuminated regions. A selected nucleotide, typically in the form of a 3′-O-phosphoramidite-activated deoxynucleoside (protected at the 5′ hydroxyl with a photosensitive protecting group), is then presented to the surface and coupling occurs at the sites that were exposed to light.

Following capping and oxidation, the substrate is rinsed and the surface is illuminated through a second mask to expose additional hydroxyl groups for coupling. A second selected nucleotide (e.g., 5′-protected, 3′-O-phosphoramidite-activated deoxynucleoside) is presented to the surface. The selective deprotection and coupling cycles are repeated until the desired set of products is obtained. Since photolithography is used the process can be readily miniaturized to generate high density arrays of oligonucleotide probes. Furthermore, the sequence of the oligonucleotides at each site is known. See Pease et al. Mechanical synthesis methods are similar to the light directed methods except involving mechanical direction of fluids for deprotection and addition in the synthesis steps.

For some embodiments, oligonucleotide arrays may be prepared having all possible probes of a given length. The hybridization pattern of the target sequence on the array may be used to reconstruct the target DNA sequence. Hybridization analysis of large numbers of probes can be used to sequence long stretches of DNA or provide an oligonucleotide array which is specific and complementary to a particular nucleic acid sequence. For example, in particularly preferred aspects, the oligonucleotide array will contain oligonucleotide probes which are complementary to specific target sequences, and individual or multiple mutations of these. Such arrays are particularly useful in the diagnosis of specific disorders which are characterized by the presence of a particular nucleic acid sequence.

Following sample collection and nucleic acid extraction, the nucleic acid portion of the sample is typically subjected to one or more preparative reactions. These preparative reactions include in vitro transcription, labeling, fragmentation, amplification and other reactions. Nucleic acid amplification increases the number of copies of the target nucleic acid sequence of interest. A variety of amplification methods are suitable for use in the methods and device of the present invention, including for example, the polymerase chain reaction method or (PCR), the ligase chain reaction (LCR), self sustained sequence replication (3SR), and nucleic acid based sequence amplification (NASBA).

The latter two amplification methods involve isothermal reactions based on isothermal transcription, which produce both single stranded RNA (ssRNA) and double stranded DNA (dsDNA) as the amplification products in a ratio of approximately 30 or 100 to 1, respectively. As a result, where these latter methods are employed, sequence analysis may be carried out using either type of substrate, i.e. complementary to either DNA or RNA.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

### Determining Transcriptional Expression Levels

Expression of genes may in general be detected by either detecting mRNA from the cells and/or detecting expression products, such as peptides and proteins.

Polymerase Chain reaction (PCR) is a well known and well established technique to determine transcriptional products and therefor also a method that in one embodiment is used to determine the transcriptional expression level of the SLC18A2 gene, or part thereof.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

Thus, in one embodiment, this invention provides for a method of optimizing a probe set for detection of a particular gene. Generally, this method involves providing a high density array containing a multiplicity of probes of one or more particular length(s) that are complementary to subsequences of the mRNA transcribed by the target gene. In one embodiment the high density array may contain every probe of a particular length that is complementary to a particular mRNA. The probes of the high density array are then hybridized with their target nucleic acid alone and then hybridized with a high complexity, high concentration nucleic acid sample that does not contain the targets complementary to the probes. Thus, for example, where the target nucleic acid is an RNA, the probes are first hybridized with their target nucleic acid alone and then hybridized with RNA made from a cDNA library (e.g., reverse transcribed polyA.sup.+mRNA) where the sense of the hybridized RNA is opposite that of the target nucleic acid (to insure that the high complexity sample does not contain targets for the probes). Those probes that show a strong hybridization signal with their target and little or no cross-hybridization with the high complexity sample are preferred probes for use in the high density arrays of this invention.

PCR amplification generally involves the use of one strand of the target nucleic acid sequence as a template for producing a large number of complements to that sequence. Generally, two primer sequences complementary to different ends of a segment of the complementary strands of the target sequence hybridize with their respective strands of the target sequence, and in the presence of polymerase enzymes and nucleoside triphosphates, the primers are extended along the target sequence. The extensions are melted from the target sequence and the process is repeated, this time with the additional copies of the target sequence synthesized in the preceding steps. PCR amplification typically involves repeated cycles of denaturation, hybridization and extension reactions to produce sufficient amounts of the target nucleic acid. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In PCR methods, strand separation is normally achieved by heating the reaction to a sufficiently high temperature for a sufficient time to cause the denaturation of the duplex but not to cause an irreversible denaturation of the polymerase. Typical heat denaturation involves temperatures ranging from about 80.degree. C. to 105.degree. C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity.

In addition to PCR and IVT reactions, the methods of the present invention are also applicable to a number of other reaction types, e.g., reverse transcription, nick translation, and the like.

The nucleic acids in a sample will generally be labelled to facilitate detection in subsequent steps. Labelling may be carried out during the amplification, in vitro transcription or nick translation processes. In particular, amplification, in vitro transcription or nick translation may incorporate a label into the amplified or transcribed sequence, either through the use of labelled primers or the incorporation of labelled dNTPs into the amplified sequence.

Hybridization between the sample nucleic acid and the oligonucleotide probes upon the array is then detected, using, e.g., epifluorescence confocal microscopy. Typically, sample is mixed during hybridization to enhance hybridization of nucleic acids in the sample to nucleic acid probes on the array.

In some cases, hybridized oligonucleotides may be labelled following hybridization. For example, where biotin labeled dNTPs are used in, e.g. amplification or transcription, streptavidin linked reporter groups may be used to label hybridized complexes. Such operations can readily be integrated into the systems of the present invention. Alternatively, the nucleic acids in the sample may be labelled following amplification. Post amplification labelling typically involves the covalent attachment of a particular detectable group upon the amplified sequences. Suitable labels or detectable groups include a variety of fluorescent or radioactive labelling groups well known in the art. These labels may also be coupled to the sequences using methods that are well known in the art.

Methods for detection of SLC18A2 transcript or part thereof depend upon the label selected. A fluorescent label is preferred because of its extreme sensitivity and simplicity. Standard labelling procedures are used to determine the positions where interactions between a sequence and a reagent take place. For example, if a target sequence is labelled and exposed to a matrix of different probes, only those locations where probes do interact with the target will exhibit any signal. Alternatively, other methods may be used to scan the matrix to determine where interaction takes place. Of course, the spectrum of interactions may be determined in a temporal manner by repeated scans of interactions which occur at each of a multiplicity of conditions. However, instead of testing each individual interaction separately, a multiplicity of sequence interactions may be simultaneously determined on a matrix.

Means of detecting labelled target (sample) SLC18A2 nucleic acids hybridized to the probes of the high density array are known to those of skill in the art. Thus, for example, where a colorimetric label is used, simple visualization of the label is sufficient. Where a radioactive labelled probe is used, detection of the radiation (e.g with photographic film or a solid state detector) is sufficient.

In a preferred embodiment, however, the target nucleic acids are labelled with a fluorescent label and the localization of the label on the probe array is accomplished with fluorescent microscopy. The hybridized array is excited with a light source at the excitation wavelength of the particular fluorescent label and the resulting fluorescence at the emission wavelength is detected. In a particularly preferred embodiment, the excitation light source is a laser appropriate for the excitation of the fluorescent label.

The target polynucleotide may be labelled by any of a number of convenient detectable markers. A fluorescent label is preferred because it provides a very strong signal with low background. It is also optically detectable at high resolution and sensitivity through a quick scanning procedure. Other potential labelling moieties include, radioisotopes, chemiluminescent compounds, labelled binding proteins, heavy metal atoms, spectroscopic markers, magnetic labels, and linked enzymes.

Another method for labelling may bypass any label of the target sequence. The target may be exposed to the probes, and a double strand hybrid is formed at those positions only. Addition of a double strand specific reagent will detect where hybridization takes place. An intercalative dye such as ethidium bromide may be used as long as the probes themselves do not fold back on themselves to a significant extent forming hairpin loops. However, the length of the hairpin loops in short oligonucleotide probes would typically be insufficient to form a stable duplex.

Suitable chromogens will include molecules and compounds which absorb light in a distinctive range of wavelengths so that a color may be observed, or emit light when irradiated with radiation of a particular wave length or wave length range, e.g., fluorescers. Biliproteins, e.g., phycoerythrin, may also serve as labels.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorescers may be employed either by themselves or in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3-chromen, indole, xanthen, 7-hydroxycoumarin, phenoxazine, salicylate, strophanthidin, porphyrins, triarylmethanes and flavin. Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4-(3′ pyrenyl)butyrate; d-3-aminodesoxy-equilenin; 12-(9′-anthroyl)stearate; 2-methylanthracene; 9-vinylanthracene; 2,2′-(vinylene-p-phenylene)bisbenzoxazole; p-bis>2-(4-methyl-5-phenyl-oxazolyl)benzene; 6-dimethylamino-1,2-benzophenazin; retinol; bis(3′-aminopyridinium) 1,10-decandiyldiiodide; sulfonaphthylhydrazone of hellibrienin; chlorotetracycline; N-(7-dimethylamino-4-methyl-2-oxo-3-chromenyl)maleimide; N->p-(2-benzimidazolyl)-phenyllmaleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye may differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal may also be provided by chemiluminescent and bioluminescent sources. Chemiluminescent sources include a compound which becomes electronically excited by a chemical reaction and may then emit light which serves as the detectible signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5-amino-6,7,8-trimethoxy- and the dimethylamino>calbenz analog. These compounds can be made to luminescence with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence may also be obtained with oxalates, usually oxalyl active esters, e.g., p-nitrophenyl and a peroxide, e.g., hydrogen peroxide, under basic conditions. Alternatively, luciferins may be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

In addition, amplified sequences may be subjected to other post amplification treatments. For example, in some cases, it may be desirable to fragment the sequence prior to hybridization with an oligonucleotide array, in order to provide segments which are more readily accessible to the probes, which avoid looping and/or hybridization to multiple probes. Fragmentation of the nucleic acids may generally be carried out by physical, chemical or enzymatic methods that are known in the art.

Following the various sample preparation operations, the sample will generally be subjected to one or more analysis operations. Particularly preferred analysis operations include, e.g. sequence based analyses using an oligonucleotide array and/or size based analyses using, e.g. microcapillary array electrophoresis.

In some embodiments it may be desirable to provide additional, or alternative means for analyzing the nucleic acids from the sample

Microcapillary array electrophoresis generally involves the use of a thin capillary or channel which may or may not be filled with a particular separation medium. Electrophoresis of a sample through the capillary provides a size based separation profile for the sample. Microcapillary array electrophoresis generally provides a rapid method for size based sequencing, PCR product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods these methods provide sensitivity in the range of attomoles, which is comparable to the sensitivity of radioactive sequencing methods.

In many capillary electrophoresis methods, the capillaries e.g. fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g. hydroxyethyl cellulose, polyacrylamide and agarose. Gel matrices may be introduced and polymerized within the capillary channel. However, in some cases this may result in entrapment of bubbles within the channels which can interfere with sample separations. Accordingly, it is often desirable to place a preformed separation matrix within the capillary channel(s), prior to mating the planar elements of the capillary portion. Fixing the two parts, e.g. through sonic welding, permanently fixes the matrix within the channel. Polymerization outside of the channels helps to ensure that no bubbles are formed. Further, the pressure of the welding process helps to ensure a void-free system.

In addition to its use in nucleic acid “fingerprinting” and other sized based analyses the capillary arrays may also be used in sequencing applications. In particular, gel based sequencing techniques may be readily adapted for capillary array electrophoresis.

Transcriptional expression products from the SLC18A2 gene may be detected as indications of prostate cancer of the sample tested, such as diagnosing, predicting, prognosing, monitoring prostate cancer in an individual. The transcriptional expression product of the SLC18A2 gene may be detected in either a tissue sample as such, or in a body fluid sample, such as blood, serum, plasma, semen and/or urine of the individual.

When in the present invention the transcriptional level of the SLC18A2 gene in a sample is decreased by at least 10%, or for example at least 15%, 20%, 25%, 30%, 35%, 40%, 41%, 42%, 43%, 44%, 45%, 46%, 47%, 48%, 49%, or at least 50% and even more, relative to previous measurements on the same individual, or to measurements on non-diseased tissue from the same individual, or relative to a standard level, the transcriptional expression level of the SLC18A2 gene is indicative of the presence of prostate cancer. When a standard transcriptional expression level has a value of 100, and the test sample has a transcriptional expression level of the SLC18A2 gene at a value of 50, the expression level is said to be decreased by 50%.

In a preferred embodiment a transcriptional expression level of SLC18A2 decreased by at least 50% is indicative of the presence of prostate cancer. In another preferred embodiment a transcriptional level of the SLC18A2 gene decreased by 2 fold or more, such as 3-fold, 4-fold, 5-fold or more is indicative of prostate cancer.

In the comparisons, the control sample is an equivalent RNA sample from one and/or a pool of RNA samples more than one healthy male without (clinically diagnosed) prostate cancer and/or from the same person being tested—but control sample was taken at an earlier time point e.g in the course of an active surveillance programs.

### Methods for Determining the Translational Expression Level of the SLC18A2 Gene

The expression products, peptides and proteins, may be detected by any suitable technique known to the person skilled in the art.

In a preferred embodiment the translational expression level of SLC18A2 or part thereof are detected by means of specific antibodies directed to the SLC18A2 protein product, such as immunofluorescent and/or immunohistochemical staining of the tissue.

Immunohistochemical localization of expressed SLC18A2 may be carried out by immunostaining of tissue sections from for example tissue samples such as a biopsy of the prostate cancer tumor to determine the level of translational expression.

For example sections may be cut from paraffin-embedded tissue blocks, mounted, and deparaffinized by incubation at 80 C.° for 10 min. followed by immersion in heated oil at 60°C. for 10 min. (Estisol 312, Estichem A/S, Denmark) and rehydration. Antigen retrieval is achieved in TEG (TrisEDTA-Glycerol) buffer using microwaves at 900 W. The tissue sections may be cooled in the buffer for 15 min before a brief rinse in tap water. Endogenous peroxidase activity is blocked by incubating the sections with 1% H202 for 20 min. followed by three rinses in tap water, 1 min each. The sections may then be soaked in PBS buffer for 2 min. The next steps can be modified from the descriptions given by Oncogene Science Inc., in the Mouse Immunohistochemistry Detection System, XHCO1 (UniTect, Uniondale, N.Y., USA). Briefly, the tissue sections are incubated overnight at 4° C. with primary antibody directed against an epitope of the SLC18A2 protein, followed by for example three rinses in PBS buffer for 5 min each. Afterwards, the sections are incubated with biotinylated secondary antibody for 30 min, rinsed three times with PBS buffer and subsequently incubated with ABC (avidin-biotinlylated horseradish peroxidase complex) for 30 min. followed by three rinses in PBS buffer.

Staining may be performed by incubation with AEC (3-amino-ethylcarbazole) for 10 min. The tissue sections are counter stained with Mayers hematoxylin, washed in tap water for 5 min. and mounted with glycerol-gelatin. Positive and negative controls may be included in each staining round with all antibodies.

In yet another embodiment the SLC18A2 protein or part thereof may be detected by means of conventional enzyme assays, such as ELISA methods.

Furthermore, the SLC18A2 protein or part thereof may be detected by means of peptide/protein chips capable of specifically binding the peptides and/or proteins assessed. Thereby an expression pattern may be obtained.

Translational expression products from the SLC18A2 gene may be detected as indications of prostate cancer of the sample tested, such as diagnosing, predicting, prognosing, monitoring prostate cancer in an individual. The translational expression products of the SLC18A2 gene may be detected in either a tissue sample as such, or in a body fluid sample, such as blood, serum, plasma, semen and/or urine of the individual. Preferably, the translational level is measured in a tissue sample, for example a biopsy, prostate tumor tissue, or a tissue of a resected prostate.

When in a tissue section following prostatectomy, staining for the presence of SLC18A2 protein is observed this is considered as an indication that the prediction of disease progression is favourable, for example that the recurrence risk is less severe. In contrast, if no staining can be identified in such a tissue section the prediction of disease progression is unfavourable.

When in the present invention the translational expression level of the SLC18A2 gene in a sample is decreased by at least 10%, or for example at least 15%, 20%, 25%, 30%, 35%, 40%, 41%, 42%, 43%, 44%, 45%, 46%, 47%, 48%, 49%, or at least 50% and even more, relative to previous measurements on the same individual, or to measurements on non-diseased tissue from the same individual, or relative to a standard level, the translational expression level of the SLC18A2 gene is indicative of the presence of prostate cancer. When a standard transcriptional expression level has a value of 100, and the test sample has a transcriptional expression level of the SLC18A2 gene at a value of 50, the expression level is said to be decreased by 50%.

In a preferred embodiment a translational expression level of SLC18A2 decreased by at least 50% is indicative of the presence of prostate cancer. In another preferred embodiment a translational level of the SLC18A2 gene decreased by 2 fold or more, such as 3-fold, 4-fold, 5-fold or more is indicative of prostate cancer.

In the comparisons, the control sample is an equivalent sample from one and/or a pool of samples from more than one healthy male without (clinically diagnosed) prostate cancer and/or from the same person being tested—but control sample was taken at an earlier time point e.g in the course of an active surveillance programs.

### Determining Treatment Regime

One aspect of the present invention relates to a method for assisting in determining and/or determining the treatment regime of an individual having contracted prostate cancer comprising the steps of i) determining the methylation status of a SLC18A2 gene (SEQ ID NO:1), or nucleotide sequence having at least 90% sequence identity with SEQ ID NO:1, or part thereof in a sample from said individual, and/or ii) determining the transcriptional and/or translational expression level of said SLC18A2 gene, or part thereof in said sample iii) comparing the methylation status of said SLC18A2 determined in step i) to the methylation status of a control sample, and/or iv) comparing the transcriptional and/or the translational expression level of said SLC18A2 gene determined in step iii) to the expression level of a control sample, wherein the comparison results of the methylation status and/or expression level is indicative of the treatment regime to be offered to the individual having contracted prostate cancer, wherein a presence of prostate cancer determined based on the previous steps of the method, results in a decision on which treatment regime the individual should be offered. The individual could be offered any of the treatments in current practise, such as surgery to remove tumor tissue or surgery in the form of radical prostatectomy, radiation therapy, hormonal treatment, chemotherapy, cryosurgery, or high intensity focused ultrasound. In one particular embodiment the individual is treated with methylation modulating agents as described elsewhere herein, either alone or in combination with any of the listed treatment regimes. Alternatively or simultaneously, the individual could receive treatment in the form supplying the pharmaceutical composition of the present invention.

### Screening for Prostate Cancer

It is within the scope of the present invention that the methods disclosed herein can be used for screening of cancer in a population. Individuals which are most likely to enter into screening programmes according to the teachings of the present invention are those being at risk to develop cancer due to family history (e.g., individuals who have a first degree or second degree relative who is/was diagnosed with cancer), individuals predisposed to cancer due to an inheritance of a mutation in gene associated with increased predisposition to cancer (e.g., p53, BRCA1, BRCA2), individuals who are at risk to develop cancer due to occupational hazard (e.g., exposure to radiation such as ionizing radiation, cellular radiation, radio-isotopes), exposure to various carcinogens, cigarette smoking and the like, and/or individuals from a certain age or body weight (e.g., above 40 years, preferably, above 50 years) which have increased risk to develop cancer due to their age or weight.

### Methods of Treatment and Uses

One aspect of the present invention pertains to a method of treatment of an individual having contracted prostate cancer comprising administration of a methylation modulating agent in a therapeutically effective amount to an individual in need thereof, wherein said individual has contracted prostate cancer as determined by the SLC18A2 marker according to the methods of the present invention.

Thus, the present invention relates to a pharmaceutical composition for treating or ameliorating prostate cancer comprising a methylation modulating agent. In another aspect the present invention relates to the use of a methylation modulating agent for the manufacture of a medicament for prostate cancer. Yet another aspect of the present invention pertains to a methylation modulating agent for treatment of prostate cancer.

The methylation modulating agent of the present invention is capable of reducing the methylation status of a SLC18A2 gene (SEQ ID NO: 1), homology and/or increasing the transcriptional and/or translational expression level of the SLC18A2 gene. The methylation modulating agent is selected from the group consisting of DNA methylation inhibitors and histone deacetylases.

Thus the methylation modulating agent is selected individually from 5-azacytidine (5-aza-CR), 5-aza-2′deoxycytidine (5-aza-CdR), 5 fluorocytosine, pseudoisocytosine, Zebularine, Procainamide, polyphenol (−)-epigallocatechin-3-gallate (EGCG), Psammaplin, Trichostatin A, trapoxin B, depsipeptides, benzamides, electrophilic ketones, phenylbutyrate, sodium butyrate and valproic acid, but also suberoylanilide hydroxamic acid (SAHA/Vorinostat), Belinostat/PXD101, MS275, LAQ824/LBH589, 01994, MGCD0103, nicotinamide, derivatives of NAD, N-nitroso-n-methylurea, dihydrocoumarin, naphthopyranone, 4-phenylbutyric acid or 2-hydroxynaphaldehydes, in any combination.

DNA methylation inhibitors are for example 5-azacytidine (5-aza-CR), 5-aza-2′deoxycytidine (5-aza-CdR), 5 fluorocytosine, pseudoisocytosine, Zebularine, Procainamide, polyphenol (−)-epigallocatechin-3-gallate (EGCG), and Psammaplin. One preferred DNA methylation inhibitor is 5-aza. DNA methylation inhibitors such as 5-aza-CR and 5-aza-CdR are converted to the deoxynucleotide triphosphates and are then incorporated in place of cytosine into replicating DNA. They are therefore active only in S-phase cells, where they serve as powerful mechanism-based inhibitors of DNA methylation.

Histone deacetylase inhibitors are compounds that interfere with histone deacetylase function that controls the coiling and uncoiling of DNA around histones. Histone deacetylases assist in the formation of a condensed, transcriptionally silenced chromatin structure. Histone deacetylase inhibitors can counteract silencing and assist in the formation of a transcriptionally active chromatin structure. Non-limiting examples of histone deacetylase inhibitors are hyroxamic acids, such as Trichostatin A, cyclic tetrapeptides, for example trapoxin B, depsipeptides, benzamides, electrophilic ketones, and aliphatic acid compounds as for example phenylbutyrate, sodium butyrate and valproic acid, but also suberoylanilide hydroxamic acid (SAHA/Vorinostat), Belinostat/PXD101, MS275, LAQ824/LBH589, 01994, MGCD0103, nicotinamide, derivatives of NAD, N-nitroso-n-methylurea, dihydrocoumarin, naphthopyranone, 4-phenylbutyric acid and 2-hydroxynaphaldehydes.

It is appreciated that at least one methylation modulating agent is a DNA methylation inhibitor may be combined with a histone deactylase inhibitor in the treatment and/or amelioration of prostate cancer according to the present invention. One preferred combination is 4-phenylbutyric acid and 5-aza.

### Method for Reducing Tumorigenicity of a Cell

Another aspect of the present invention relates to a method of treatment which involves the introduction of a non-methylated SLC18A2 gene promoter, transcripts and/or translational product of SLC18A2 into a target cell to overcome the effect of hypermethylation of the endogenous SLC18A2 gene in the target cell. Thus, the present invention relates to a method for reducing tumorigenicity of a cell, said method comprising the steps of i) providing a) at least one SLC18A2 gene (SEQ ID NO:1), or nucleotide sequence having at least 90% sequence identity with SEQ ID NO:1, or part thereof, b) at least one SLC18A2 gene transcript (SEQ ID NO: 2), or nucleotide sequence having at least 90% sequence identity with SEQ ID NO:2, or part thereof, and/or c) at least one a translational product of the SLC18A2 gene (SEQ ID NO: 3), or nucleotide sequence having at least 90% sequence identity with SEQ ID NO:3, or part thereof; ii) introducing at least one of a), b), and/or c) of step i) into the tumor cell.

It is appreciated that at least one of a), b) and/or c) may be introduced into the tumor cell, such as two, three, four, 5, 6, 7, 8, 9, 10, 15, 20 or more of a), b), and/or c), in any combination.

The genetic material discussed above may be any of the described genes or functional parts thereof. The constructs may be introduced as a single DNA molecule encoding all of the genes, or different DNA molecules having one or more genes. The constructs may be introduced simultaneously or consecutively, each with the same or different markers.

The gene may be linked to the complex as such or protected by any suitable system normally used for transfection such as viral vectors or artificial viral envelope, liposomes or micellas, wherein the system is linked to the complex.

Numerous techniques for introducing DNA into eukaryotic cells are known to the skilled artisan. Often this is done by means of vectors, and often in the form of nucleic acid encapsidated by a (frequently virus-like) proteinaceous coat. Gene delivery systems may be applied to a wide range of clinical as well as experimental applications.

Vectors containing useful elements such as selectable and/or amplifiable markers, promoter/enhancer elements for expression in mammalian, particularly human, cells, and which may be used to prepare stocks of construct DNAs and for carrying out transfections are well known in the art. Many are commercially available.

Various techniques have been developed for modification of target tissue and cells in vivo. A number of virus vectors, discussed below, are known which allow transfection and random integration of the virus into the host. See, for example, Dubensky et al. (1984) Proc. Natl. Acad. Sci. USA 81:7529-7533; Kaneda et al., (1989) Science 243:375-378; Hiebert et al. (1989) Proc. Natl. Acad. Sci. USA 86:3594-3598; Hatzoglu et al., (1990) J. Biol. Chem. 265:17285-17293; Ferry et al. (1991) Proc. Natl. Acad. Sci. USA 88:8377-8381. Routes and modes of administering the vector include injection, e.g intravascularly or intramuscularly, inhalation, or other parenteral administration.

Advantages of adenovirus vectors for human gene therapy include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organism.

Another vector which can express the DNA molecule of the present invention, and is useful in gene therapy, particularly in humans, is vaccinia virus, which can be rendered non-replicating (U.S. Pat. Nos. 5,225,336; 5,204,243; 5,155,020; 4,769,330).

Based on the concept of viral mimicry, artificial viral envelopes (AVE) are designed based on the structure and composition of a viral membrane, such as HIV-1 or RSV and used to deliver genes into cells in vitro and in vivo. See, for example, U.S. Pat. No. 5,252,348, Schreier H. et al., J. Mol. Recognit., 1995, 8:59-62; Schreier H et al., J. Biol. Chem., 1994, 269:9090-9098; Schreier, H., Pharm. Acta Helv. 1994, 68:145-159; Chander, R et al. Life Sci., 1992, 50:481-489, which references are hereby incorporated by reference in their entirety. The envelope is preferably produced in a two-step dialysis procedure where the “naked” envelope is formed initially, followed by unidirectional insertion of the viral surface glycoprotein of interest. This process and the physical characteristics of the resulting AVE are described in detail by Chander et al., (supra). Examples of AVE systems are (a) an AVE containing the HIV-1 surface glycoprotein gp160 (Chander et al., supra; Schreier et al., 1995, supra) or glycosyl phosphatidylinositol (GPI)-linked gp120 (Schreier et al., 1994, supra), respectively, and (b) an AVE containing the respiratory syncytial virus (RSV) attachment (G) and fusion (F) glycoproteins (Stecenko, A. A. et al., Pharm. Pharmacol. Lett. 1:127-129 (1992)). Thus, vesicles are constructed which mimic the natural membranes of enveloped viruses in their ability to bind to and deliver materials to cells bearing corresponding surface receptors.

AVEs are used to deliver genes both by intravenous injection and by instillation in the lungs. For example, AVEs are manufactured to mimic RSV, exhibiting the RSV F surface glycoprotein which provides selective entry into epithelial cells. F-AVE are loaded with a plasmid coding for the gene of interest, (or a reporter gene such as CAT not present in mammalian tissue).

The AVE system described herein in physically and chemically essentially identical to the natural virus yet is entirely “artificial”, as it is constructed from phospholipids, cholesterol, and recombinant viral surface glycoproteins. Hence, there is no carry-over of viral genetic information and no danger of inadvertant viral infection. Construction of the AVEs in two independent steps allows for bulk production of the plain lipid envelopes which, in a separate second step, can then be marked with the desired viral glycoprotein, also allowing for the preparation of protein cocktail formulations if desired.

Another delivery vehicle for use in the present invention are based on the recent description of attenuated Shigella as a DNA delivery system (Sizemore, D. R. et al., Science 270:299-302 (1995), which reference is incorporated by reference in its entirety). This approach exploits the ability of Shigellae to enter epithelial cells and escape the phagocytic vacuole as a method for delivering the gene construct into the cytoplasm of the target cell. Invasion with as few as one to five bacteria can result in expression of the foreign plasmid DNA delivered by these bacteria.

A preferred type of mediator of nonviral transfection in vitro and in vivo is cationic (ammonium derivatized) lipids. These positively charged lipids form complexes with negatively charged DNA, resulting in DNA charged neutralization and compaction. The complexes endocytosed upon association with the cell membrane, and the DNA somehow escapes the endosome, gaining access to the cytoplasm. Cationic lipid:DNA complexes appear highly stable under normal conditions. Studies of the cationic lipid DOTAP suggest the complex dissociates when the inner layer of the cell membrane is destabilized and anionic lipids from the inner layer displace DNA from the cationic lipid. Several cationic lipids are available commercially. Two of these, DMRI and DC-cholesterol, have been used in human clinical trials. First generation cationic lipids are less efficient than viral vectors. For delivery to lung, any inflammatory responses accompanying the liposome administration are reduced by changing the delivery mode to aerosol administration which distributes the dose more evenly.

### Pharmaceutical Composition

The invention also relates to a pharmaceutical composition for treating prostate cancer. Thus, in one aspect of the present invention the pharmaceutical composition comprises i) at least one SLC18A2 gene (SEQ ID NO:1), or nucleotide sequence having at least 90% sequence identity with SEQ ID NO:1, or part thereof, ii) at least one SLC18A2 gene transcript (SEQ ID NO: 2), or nucleotide sequence having at least 90% sequence identity with SEQ ID NO:2, or part thereof, and/or iii) at least one translational product of the SLC18A2 gene (SEQ ID NO: 3), or nucleotide sequence having at least 90% sequence identity with SEQ ID NO:3, or part thereof.

In a preferred embodiment the gene, transcript and/or translational product of the SLC18A2 gene are bound to carriers. The peptides may be coupled to a polymer carrier, for example a protein carrier such as BSA. Such formulations are well known to the skilled person.

The present invention also relates to a compound comprising i) at least one SLC18A2 gene (SEQ ID NO:1), or nucleotide sequence having at least 90% sequence identity with SEQ ID NO:1, or part thereof, ii) at least one SLC18A2 gene transcript (SEQ ID NO: 2), or nucleotide sequence having at least 90% sequence identity with SEQ ID NO:2, or part thereof, and/or iii) at least one translational product of the SLC18A2 gene (SEQ ID NO: 3), or nucleotide sequence having at least 90% sequence identity with SEQ ID NO:3, or part thereof for treatment and/or amelioration of prostate cancer.

In the present context the term pharmaceutical composition is used synonymously with the term medicament. The medicament of the invention comprises an effective amount of one or more of the compounds as defined above, or a composition as defined above in combination with pharmaceutically acceptable additives. Such medicament may suitably be formulated for oral, percutaneous, intramuscular, intravenous, intracranial, intrathecal, intracerebroventricular, intranasal or pulmonal administration. For most indications a localised or substantially localised application is preferred.

Strategies in formulation development of medicaments and compositions based on the compounds of the present invention generally correspond to formulation strategies for any other protein-based drug product. Potential problems and the guidance required to overcome these problems are dealt with in several textbooks, e.g. “Therapeutic Peptides and Protein Formulation. Processing and Delivery Systems”, Ed. A. K. Banga, Technomic Publishing AG, Basel, 1995.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, or which enhance the effectiveness or transportation of the preparation.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are of the order of several hundred μg active ingredient per administration with a preferred range of from about 0.1 μg to 1000 μg, such as in the range of from about 1 μg to 300 μg, and especially in the range of from about 10 μg to 50 μg. Administration may be performed once or may be followed by subsequent administrations. The dosage will also depend on the route of administration and will vary with the age and weight of the subject to be treated. A preferred dosis would be in the interval 30 mg to 70 mg per 70 kg body weight.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promotes delivery of the active substance to its target.

In many instances, it will be necessary to administrate the formulation multiple times. Administration may be a continuous infusion, such as intraventricular infusion or administration in more doses such as more times a day, daily, more times a week, weekly, etc.

### Kit

According to another aspect of the present invention, it provides a kit (assay) useful for detecting prostate cancer and various aspects of prostate cancer, e.g., using the methods disclosed herein.

Thus, the present invention relates to a kit (assay) comprising at least one detection member for a SLC18A2 gene, transcriptional and/or translational product or part thereof for use in the methods of the present invention: for assisting in the diagnosis and/or for diagnosing of prostate cancer, for assisting in the prognosis and/or for the prognosis of the disease progression of prostate cancer, for assisting in the prediction and/or the prediction of the progression of prostate cancer, for assisting in predicting and/or for predicting the recurrence risk of prostate cancer, for assisting in monitoring and/or for monitoring the effect of treatment on prostate cancer progression, for assisting in monitoring and/or for monitoring the progression of prostate cancer from a silent to an aggressive prostate cancer, for assisting in determining and/or determining the treatment regime.

In one embodiment, the at least one detection member, such as two, three, four, 5, 6, 7, 8, 9, 10 or more detection members is an antibody directed against an epitope of the translational product of the SLC18A2 gene, i.e. SEQ ID NO.:3, or part thereof, oligonucleotides, primers and/or probes that are able to hybridise to the SLC18A2 gene and/or the SLC18A2 transcript.

When the at least one detection member is an antibody directed against an epitope of the SLC18A2 translational product, the antibody is monoclonal, polyclonal, or a mixture of at least two monoclonal antibodies.

Detection members are also one or more probes and/or oligonucleotides that are able to hybridise to the SLC18A2 gene or transcript thereof, that is to SEQ ID NO.:1, SEQ ID NO.:2, SEQ ID NO.:4 and/or SEQ ID NO.:5 or their complement, variant or parts thereof as described elsewhere herein.

In one embodiment the assay further comprises means for providing the level and/or means for providing informations as to whether the level is above or below a cut off value. In the present context the level here refers to methylation status as methylation level, transcriptional expression level and/or translational expression level of the SLC18A2 gene.

In one embodiment, the present invention provides a kit, e.g., a compartmentalized carrier including a first container containing a pair of primers for amplification of the sample SLC18A2 gene, a optionally a second container containing a pair of primers for amplification of a region in a reference gene, and a third container containing a first and second oligonucleotide probe specific for the amplification of the SLC18A2 gene and the region of the reference gene, respectively.

In another embodiment, the kit provided by the present invention further includes a fourth container containing a modifying agent that modifies unmethylated cytosine to produce a converted nucleic acid, e.g., uracil. Any suitable modifying agent, such as an agent that modifies unmethylated cytosine nucleotides, can be included in the kit provided by the present invention. For example, the modifying agent can be sodium bisulfite.

The kit may also comprise additional reagents used in the amplifying step of the detection method as disclosed herein. Thus, the kit may further comprise deoxyribonucleoside triphosphates, DNA polymerase enzyme and/or nucleic acid amplification buffer.

In another embodiment, the present invention provides a kit for example a compartmentalized carrier including a first container comprising an antibody directed against a SLC18A2 translation product or part thereof, and a second container containing a reference SLC18A2 protein.

In yet another embodiment, the present invention provides a kit as for example a compartmentalized carrier including a first container comprising at least one oligo nucleotide or primer pair able to hybridise to the SLC18A2 transcript, or DNA derived therefrom, and a second container comprising a control sample.

The kit may in preferred embodiments further comprise instructions for the performance of the detection method of the kit and for the interpretation of the results. The kit involves the method of detecting the methylation status of a CpG-containing nucleic acid, wherein said CpG-containing nucleic acids is modified using an agent which modifies at least one unmethylated cytosine in said methylated CpG-containing nucleic acid and amplifying said CpG-containing nucleic acid by means of at least one methylation-independent oligonucleotide primer. The instructions for performing the method of the kit comprises for example information of particular annealing temperatures to be used for the at least one methylation-independent primers, as well as for example information on cycling parameters. The kit may further comprise instructions for the interpretation of the results obtained by the method. For example how to interpret the amplified products subsequently analysed by high resolution melting analysis or methods as described elsewhere herein. For example the kit in one embodiment comprises means for providing a methylation level and/or means for providing information as to determine whether the level of methylation is above or below a cut off value. However, the assay in another embodiment comprises means for providing a transcriptional and/or translational expression level, and/or means for providing information as to determine whether the level of expression is above or below a cut off value.

The kit may in preferred embodiments further comprise software comprising an algorithm for calculation of primer annealing temperature and interpretation of results.

In yet another embodiment, the kit provided by the present invention further includes a probe for PSA determination. In still another embodiment, the kit provided by the present invention further includes an instruction insert disclosing normal and/or abnormal methylation ratio ranges for the detection of neoplasia, describing the types of samples suitable or unsuitable for the application of the kit, and/or the specificity or sensitivity provided by the assays utilizing the kit of the present invention.

According to one embodiment of the present invention, the kit provided by the present invention includes a first container containing at least one pair of primers for amplification of a promoter region of SLC18A2, a second container containing at least one pair of primers for amplification of a region of a reference gene, and a third container containing a first and second oligonucleotide probe specific to the amplification of the promoter region of SLC18A2 and the region of the reference gene, respectively, provided that one or both primers for amplification of the promoter region of SLC18A2 or one or more first oligonucleotide probes specific to the amplicon of the promoter region of SLC18A2 are capable of distinguishing between methylated and unmethylated nucleic acid, either directly or indirectly, e.g., after bisulfite modification. Optionally the kit provided by this embodiment of the present invention can further include an instruction insert, e.g., disclosing the cut off values to be consulted for determining prostate adenocarcinoma or that the kit can be used with a prostate cancer tissue sample, e.g., most suitable to be used with a prostate tissue sample.

The present invention also provides a kit useful for detecting prostate adenocarcinoma, for example in body fluid samples. The kit includes a first container containing at least one pair of primers capable of distinguishing between methylated and unmethylated nucleic acid for amplification of a promoter region of SLC18A2 and an instruction insert disclosing, among other things, that the kit is useful for detecting prostate cancer in a body fluid sample of an individual and that a methylation level of the promoter region of SLC18A2 as determined by conventional or non-real-time PCR using the primers provided that is higher than the methylation level of the promoter region of SLC18A2 in a normal subject is indicative of prostate cancer in the subject.

For example the kit in one embodiment comprises means for providing a methylation level and/or means for providing information as to determine whether the level of methylation is above or below a cut off value, and thus indicative of the presence or absence of prostate cancer, respectively.

The present invention also relates to the use of an antibody directed against an epitope of the SLC18A2 protein or part thereof in the detection of the translational expression level of a SLC18A2 gene, or part thereof
viii) for assisting in the diagnosis and/or for diagnosing of prostate cancer,
ix) for assisting in the prognosis and/or for the prognosis of the disease progression of prostate cancer,
x) for assisting in the prediction and/or the prediction of the progression of prostate cancer,
xi) for assisting in predicting and/or for predicting the recurrence risk of prostate cancer,
xii) for assisting in monitoring and/or for monitoring the effect of treatment on prostate cancer progression,
xiii) for assisting in monitoring and/or for monitoring the progression of prostate cancer from a silent to an aggressive prostate cancer,
xiv) for assisting in determining and/or determining the treatment regime

As used herein, “consisting essentially of” does not exclude materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

### EXAMPLES

Below are non-limiting examples of the present invention in various embodiments. The examples below may thus be regarded as preferred embodiments of the present invention.

SLC18A2 was identified as a new common target gene for CpG island hypermethylation and loss of heterozygosity in prostate cancer. By bisulfite sequencing, SLC18A2 was found to be hypermethylated in nearly 90% of prostate cancers, indicating it is one of the most frequently methylated genes in this malignancy. Using prostate cancer tissue microarrays representing >700 patients from Denmark and Switzerland, SLC18A2 was significantly downregulated in adenocarcinoma compared to non-malignant prostate tissues, thereby confirming for the first time at the SLC18A2 protein level, microarray expression profiling data from our group (20) and several others (21-25). Moreover, loss of cytoplasmic SLC18A2 immunoreactivity was identified as a novel adverse predictor of biochemical recurrence after radical prostatectomy, which was independent of the well-established predictive factors preoperative PSA, Gleason score, tumor stage, and surgical margin status.

### Tissue and Samples

### Prostate Cancer Tissue Microarray (TMA)

TMAs contained 738 formalin-fixed, paraffin-embedded prostate tissues (from Institute of Surgical Pathology, University of Zurich, Zurich, Switzerland, and Institute of Pathology, Aarhus University Hospital, Aarhus, Denmark), including 506 consecutive radical prostatectomy specimens, 41 hormone-refractory prostate cancer samples, 36 lymph node metastases, 28 distant metastases (bone, lung, urinary bladder), 65 benign prostatic hyperplasias, 19 non-malignant prostate tissues from patients with prostate cancer, 15 high-grade prostatic intraepithelial neoplasias, and 28 primary tumor specimens from patients with metastatic prostate cancer. Prior to TMA construction, H&E-stained slides of all specimens were evaluated by experienced pathologists to identify representative areas, and tumor stage and Gleason score were reassigned according to UICC and WHO/ISUP criteria (26). Clinical follow-up data was available for 464 prostatectomy patients (91.7%) with a median follow-up period of 72 months (range 1-167 months). The local Scientific Ethics Committees in both countries approved the study. TMA construction has been described previously in details (27); only blocks 1 and 3 of the published Danish TMA (28) were used.

### Statistical Analyses of Tissue Microarray Data

SPSS version 16.0 (SPSS, Chicago, Ill., USA) was used for statistical analyses; p-values <0.05 were considered significant. Contingency table analysis, two-sided Fisher's exact tests, and chi-square (χ²) tests were used to study statistical associations between clinicopathological and immunohistochemical data. Time to PSA recurrence (cutoff≧0.1 ng/ml) and time to death were selected as end points. For survival analysis, only patients with primary prostate cancer undergoing radical prostatectomy were included (clinicopathological data in FIG. 8). Recurrence-free survival (RFS) and overall survival (OS) curves were calculated by the Kaplan-Meier method and evaluated by two-sided log-rank statistics. For RFS analysis, patients were censored at the time of their last tumor-free clinical follow-up visit; patients not reaching PSA nadir (<0.1 ng/ml) postoperatively were censored at PSA recurrence. For OS analysis, patients were censored at the time of their last clinical follow-up visit. A stepwise multivariable Cox regression model was adjusted, testing the independent prognostic relevance of SLC18A2 immunoreactivity. The limit for reverse selection was p=0.01. The proportionality assumption for all variables was assessed with log-negative-log survival distribution functions.

RNA Preparation, cDNA Synthesis and Quantitative RT-PCR

Total RNA from cultured cells and prostate tissue samples was isolated with the RNeasy MinElute Cleanup Kit (Qiagen), as described elsewhere (20). First-strand cDNA synthesis was performed with SuperScript II Reverse Transcriptase (Invitrogen) using oligo(dT) priming. SLC18A2 expression was measured with TaqMan Gene Expression Assay Hs00161858_m1 and TaqMan Universal PCR Master Mix on a real-time ABI PRISM 7500 Sequence Detection System (all from Applied Biosystems). For normalization, UBC expression was measured using primers 5′-GATTTGGGTCGCGGTTCTT-3′ plus 5′-TGCCTTGACATTCTCGATGGT-3′ and SYBR GREEN PCR Master Mix (Applied Biosystems). All reactions were run in triplicates.

### Example 1

### Identification of Novel Candidate Markers of Prostate Cancer

### Candidate Gene Selection

To identify novel candidate genes downregulated in prostate cancer, we re-analyzed microarray expression profiling (20) and SNP array (31) data sets generated earlier in our group. FIG. 1 lists the top 20 most significantly downregulated genes in prostate cancer versus adjacent non-malignant prostate tissue samples based on Affymetrix Exon Array analysis. Most of these genes have also been found downregulated in prostate cancer by traditional 3′ array analysis, confirming the usefulness of exon arrays for transcript level expression analysis. Two novel downregulated genes were identified: DCHS2 and VIT. Three of the 20 genes were located at common (>20%) LOH regions mapped by Affymetrix 50K SNP array analysis: CTSB at 8p22 (50% LOH), ALOX158 at 17p13.1 (23% LOH), and SLC18A2 at 10q25 (23% LOH), indicating that these genes are selectively lost in prostate cancer cells. Whereas possible roles in prostate cancer have been reported for CTSB (32) and ALOX15B (33), this remains unexamined for SLC18A2, which we selected for further investigation. SLC18A2 expression levels determined by exon array analysis were successfully validated by quantitative RT-PCR (FIG. 2A). Our findings corroborate expression profiling results for SLC18A2 in prostate cancer from several other studies using distinct microarray platforms (21-25).

### Example 2

### SLC18A2 Protein Expression in Non-Malignant and Prostate Cancer Tissue

### Tissue Microarray Analysis

SLC18A2 protein expression in non-malignant and prostate cancer tissue samples was investigated by immunohistochemical analysis of two tissue microarrays (TMAs) containing 738 specimens from Danish and Swiss patients with benign or malignant prostatic disease.

For immunohistochemistry, TEG buffer was used for epitope demasking. TMAs were stained with a polyclonal SLC18A2 (AB1767, Chemicon) antibody diluted 1:300 in TBS buffer with 1% BSA. The anti-rabbit EnVision+System (Dakocytomation, Denmark) with HRP-labelled polymer and DAB solution (Kem-En-Tec, Denmark) was used for secondary staining. For negative controls, primary antibody was omitted. Blinded scoring for cytoplasmic and nuclear SLC18A2 immunoreactivivity (0=no; 1+=weak; 2+=moderate; 3+=strong) was performed by a trained pathologist and a scientist with extensive experience in prostate histology. In cases of disagreement, cores were re-evaluted to obtain consensus. Kappa statistics showed good interobserver agreement (0.73 for cytoplasmic and 0.9 for nuclear SLC18A2 staining). Lost specimens and cores without epithelial cell content were excluded from analysis. AB1767 antibody specificity was validated by staining of a human multi-tissue array (T8235713-5; BioChain Institute Inc, Hayward, Calif., USA), which showed the expected SLC18A2 expression patterns (not shown).

The specificity of the commercial SLC18A2 antibody has been thoroughly validated by several users (7-9) and was further tested here by Western blotting analysis.

For Western blotting, Protein extracts from cultured cells and fresh-frozen BPH specimens prepared in RIPA lysis buffer were analyzed on 12% NUPAGE polyacrylamid gels (Invitrogen), blotted to PVDF membranes (Immobilon-P Transfer Membrane; Millipore), and blocked with 5% skimmed milk in PBS buffer with 0.1% Tween-20. Primary antibody (AB1767) was diluted 1:500 and secondary antibody (HRP-conjugated swine anti-rabbit immunoglobulins; Dako A/S, Denmark) 1:5000 in PBS plus 0.5% Tween-20. For visualization, we used the ECL plus WB Detection System (Amersham Biosciences). Actin-beta was used as internal control (29). Lysate from COS7 cells transfected with an expression vector for human SLC18A2 (gift from Dr. Arnold Ruoho, University of Wisconsin, Madison, Wis., USA) was used as positive control. This vector encodes a glycosylation mutant of SLC18A2 with HA and Flag/His tags (30). As negative control, COS7 cells were mock transfected with empty vector (pcDNA3.1-Flag/His; Invitrogen).

Three immunoreactive bands of the expected sizes, corresponding to glycosylated, partially glycosylated, and unglycosylated SLC18A2 protein (34), were detected in BPH tissue samples (FIG. 2B), which by real-time RT-PCR analysis expressed SLC18A2 at high levels (not shown). The antibody also detected ectopically expressed SLC18A2 protein in transfected COS7 cells (FIG. 2B). No other significant bands were detected in any of the samples, indicating high SLC18A2 antibody specificity. After staining of the TMAs, 664 out of 738 cores (90%) could be evaluated for cytoplasmic, respectively, nuclear SLC18A2 immunoreactivity (staining patterns are summarized in FIG. 2C-D), using a scale from 0 to 3 (score 0=negative, score 1+=weak; score 2+=moderate; score 3+=strong staining) (FIG. 3, panels a-d). The remaining cores (10%) were either lost or excluded from analysis due to poor technical quality or high stromal cell content.

Moderate/strong cytoplasmic SLC18A2 staining of secretory epithelial cells was observed in most (80%) BPH (FIG. 3, panel e) and adjacent non-malignant samples (FIG. 2C), whereas most (>90%) cancer samples showed no/weak cytoplasmic staining (FIG. 3, panels f-g) (p<0.001; χ² test). Thus, SLC18A2 is downregulated at the protein level in prostate cancer, confirming RNA expression profiling results. Cytoplasmic immunoreactivity was oriented towards the glandular lumen (FIG. 3, panel h), consistent with SLC18A2 localization in secretory vesicle membranes with its immunoreactive peptide extruding into the cytoplasm (35). Cytoplasmic SLC18A2 expression was higher in high-grade prostatic intraepithelial neoplasia (HG-PIN) lesions (FIG. 2C; FIG. 3, panel i) than in prostate cancer samples. However, five out of 12 HG-PIN samples showed no/weak cytoplasmic staining (FIG. 3, panels j-k), suggesting that SLC18A2 loss in some cases occur already in precancerous lesions. No differences in cytoplasmic SLC18A2 staining patterns were observed in localized versus metastatic prostate cancer, hormone-refractory prostate cancer or metastases (FIG. 2C), consistent with SLC18A2 loss being a relatively early event. Cytoplasmic SLC18A2 staining was also detected in prostate neuroendocrine cells (FIG. 3, panel l) known to secrete serotonine (15).

Nuclear SLC18A2 immunoreactivity was observed in some specimens, most predominantly in metastatic disease types (FIG. 2D; FIG. 3, panels m-n) (p<0.001; χ² test). Aberrant protein glycosylation, as demonstrated for other proteins in prostate cancer (36), has been associated with changes in intracellular SLC18A2 trafficking (37). The nuclear staining is therefore likely to represent mislocated SLC18A2 protein.

### Example 3

Methylation Status of the 5′ End of SLC18A2

SLC18A2 is hypermethylated in prostate cancer Genomic bisulfite sequencing of a CpG island at the 5′ end of SLC18A2 (FIG. 5A) was used to investigate, if SLC18A2 silencing in prostate cancer cells involves DNA hypermethylation.

For bisulfite sequencing, genomic DNA from prostate cell lines and carefully selected 20-μm sections of fresh-frozen Tissue-tek-embedded BPH, adenocarcinoma and adjacent non-malignant prostate tissue samples was isolated using the PUREGENE DNA Purification Kit (Gentra Systems) with proteinase K treatment (100 units, 30 min, 37 C), as previously described (29). Laser-microdissection was performed as described in (31). DNA was bisulfite converted using the MethylEasy DNA Bisulfite Modification Kit (Human Molecular Signaling, Sydney, Australia). The SLC18A2 promoter CpG island was PCR amplified with TEMPase Hot Start DNA Polymerase (Ampliqon) using primers 5′-TTTTAGGTTTGGGTTTTTAAGGTATT-3′ and 5′-AACTCTAAAAACCTCCCTACCTCCCTAC-3′. Gel purified (illustra GFX PCR DNA and Gel Band Purification Kit; GE Healthcare) amplicons were subcloned into the pCR4-TOPO vector (Invitrogen) and several clones sequenced using M13 forward and reverse primers. Clinicopathological data from patients undergoing methylation analysis is compiled in FIG. 4.

Bisulfite sequencing revealed dense (52-88%) hypermethylation of SLC18A2 in 5/5 prostate adenocarcinoma cell lines and in PSK-1 prostate small cell carcinoma cells, as well as low density (17%) methylation in BPH-1 benign prostatic hyperplasia cells. Essentially no methylation was seen in H69 small cell lung carcinoma (SCLC) cells (FIG. 5B), one of only a few cell lines known to express SLC18A2 (9). Real-time PCR analysis showed several fold lower SLC18A2 expression in 9/9 prostate cell lines compared to H69 (FIG. 5C), indicating that SLC18A2 silencing is mediated by CpG island hypermethylation in cultured prostate cancer cell lines.

In clinical samples, SLC18A2 was virtually unmethylated in 4/4 adjacent non-malignant prostate glands and in 3/5 BPH samples, whereas the remaining 2/5 BPHs had dense monoallelic hypermethylation (FIG. 6A). Dense SLC18A2 hypermethylation was detected in 15/17 (88%) prostate cancer samples, whilst 2/17 (12%) cancer samples were moderately methylated (PC-21 and PC-45) (FIG. 6B). Immunohistochemistry data available for ten of the cancer samples showed no/weak cytoplasmic SLC18A2 staining in nine cases, which also had SLC18A2 densely hypermethylated, while one tumor (PC-21) showed marked cytoplasmic staining and low-density methylation (FIG. 4, FIG. 6B). This strongly indicates that SLC18A2 is epigenetically silenced in prostate cancer by frequent CpG island hypermethylation. Intriguingly, two tumors (PC-23 and PC-510) displayed dense SLC18A2 hypermethylation and marked nuclear SLC18A2 staining. This inconsistency, however, could be due to tumor tissue heterogeneity. The other tumor with low-density methylation (PC-45) was not analyzed by IHC.

Scarcity of unmethylated clones in most tumors indicated that hypermethylation generally affected both alleles of SLC18A2, as confirmed for three heterozygous tumors without LOH (PC-01, PC-09, and PC-20; FIG. 6B). Biallelic hypermethylation was also seen in a crudely-dissected tumor with LOH(PC-41), suggesting that SLC18A2 hypermethylation preceded allelic loss in this case. Furthermore, laser-microdissection of eight tumors proved that SLC18A2 hypermethylation was present in prostate adenocarcinoma cells and not caused by contaminating inflammatory or stromal cells.

### Example 4

Effect of Treatment with DNA Methylation Modulators on SLC18A2 Expression

### Cell Culture and Epigenetic Drug Treatment

All cell lines were grown in RPMI 1640 with L-glutamin (Gibco, Invitrogen) supplemented with 10% fetal bovine serum, 100 U/ml penicillin and 100 μg/ml streptomycin. LNCaP, PC-3, DU145, and 22rv1 cells were obtained from ATCC, BPH-1 cells from DSMZ (Braunschweig, Germany), and PNT1A cells from ECACC. PSK-1 cells were kindly provided by Dr. Adrie van Boekhoven (University of Colorado), VCaP and DuCaP cells by Dr. Kenneth Pienta (University of Michigan), and H69 and H69A cells by Dr. Marie Stampe Ostenfeld (Danish Cancer Society, Copenhagen, Denmark). Cell lines were treated with 1 μM 5-aza-2′-deoxycytidine (Sigma) for 48 h and allowed 5 days recovery in complete medium. Four cell lines were given a combination of 1 μM 5-aza-2′-deoxycytidine (48 h treatment plus 5 days recovery) and 1 mM 4-phenylbutyric acid (Sigma) (continuously for 7 days). Mock treated cells were given buffer only. Growth media were changed daily and cells harvested on day 7. All experiments were performed in duplicate and repeated 2-3 times.

### Induction of SLC18A2 Expression by Drug Treatment

Treatment with DNA methylation inhibitor 5-aza-2′-deoxycytidine (5-aza) induced SLC18A2 expression in PSK-1 and H69 cells (FIG. 5C). This was accompanied by CpG island demethylation in PSK-1 cells from 69% to 11%, but most probably indirect in H69 cells (FIG. 7). Such indirect induction was not observed in the derived adherent line H69A, where SLC18A2 was unmethylated despite low expression (FIG. 5B-C). In SCLC cells, SLC18A2 expression therefore seems to be regulated by other mechanisms than DNA methylation of its promoter associated CpG island. Treatment of four prostate cell lines with 5-aza plus histone deacetylase inhibitor 4-phenylbutyric acid (PBA) induced SLC18A2 expression markedly in DU145 and PC3 adenocarcinoma cells, and less strongly in BPH-1 cells (FIG. 5C). SLC18A2 induction was associated with CpG island demethylation (52% to 9%) in PC3 cells, whereas methylation intensities remained unchanged in BPH-1 cells (FIG. 7). Low-density DNA methylation in BPH-1 cells therefore appeared to be permissive for moderate induction of SLC18A2, possibly through histone acetylation changes caused by PBA. The combined drug treatment scheme did not effectively demethylate (FIG. 7) nor induce expression of SLC18A2 in LNCaP cells (FIG. 5C).

### Example 5

### Predictive Value of SLC18A2 Immunoreactivity

### Univariate and Multivariate Survival Analyses

To investigate the predictive value of SLC18A2 immunoreactivity for patient outcome, we performed PSA-based recurrence-free survival (RFS) and overall survival (OS) analyses for 506 patients with primary prostate cancer undergoing radical prostatectomy (158 from Denmark and 348 from Switzerland represented on the TMAs) (See FIG. 8 for clinicopathological data). By univariate analysis, short RFS times were significantly associated with the established indicators high PSA, high Gleason score, high T stage, positive nodal, and positive surgical margin status (Table 1, FIG. 9), strongly indicating that this cohort is representative. Fisher's Exact test showed no clear associations between SLC18A2 immunoreactivity (positive vs. negative) in prostatectomy tumor specimens and any of these indicators (FIG. 10). However, when Gleason scores were instead compared to SLC18A2 intensity scores, absence of strong/moderate cytoplasmic staining was significantly associated with higher Gleason score (p=0.029; χ² test; FIG. 11).

Using log-rank statistics, negative cytoplasmic SLC18A2 immunoreactivity was significantly associated with PSA recurrence (FIG. 12A). Estimated mean RFS time for patients negative (score 0) for cytoplasmic SLC18A2 was 72 months (95% confidence interval: 60-84 months) compared to 115 months (95% confidence interval: 106-125 months) for positive (scores 1+ to 3+) cases (p<0.001). The difference was also significant between patients with negative and weak (score 1+) cytoplasmic staining (p<0.001). We note that negative cytoplasmic staining was related to shorter RFS times in patients with Gleason 7 tumors (p=0.009; FIG. 12B), a large histological subgroup for which outcome prediction is particularly difficult. This was also the case, when leaving out Gleason 7 cases (p<0.001; not shown). Nuclear SLC18A2 staining was not associated with RFS (p=0.416; FIG. 12B).

Likewise, no significant associations were found between OS and cytoplasmic (p=0.399) or nuclear (p=0.072) SLC18A2 staining (FIG. 5C). Short OS was significantly associated only with high Gleason score (p=0.001) and high tumor stage (p=0.023) (Table 1, FIG. 13).

TABLE 1
Univariate and multivariate survival analyses
Univariate analysis (Recurrence-free survival and overall survival)

| | | | Recurrence-free survival* | | | | Overall survival^{†} | | | |
|---|---|---|---|---|---|---|---|---|---|---|
| Variable | Characteristics | | n | events | Censored | Log-rank^{‡} | n | events | censored | Log-rank^{‡} |
| Age at diagnosis | <63 | years | 176 | 63 | 113 | 0.986 | 206 | 20 | 186 | 0.329 |
| (grouped) | ≧63 | years | 202 | 72 | 130 | | 248 | 34 | 214 | |
| Gleason score | 5-6 | | 112 | 16 | 96 | <0.001 | 112 | 5 | 107 | 0.001 |
| (grouped) | 7 | | 210 | 81 | 129 | | 248 | 25 | 223 | |
| | 8-10 | | 63 | 39 | 24 | | 101 | 25 | 76 | |
| Tumor stage | pT2a-c | | 257 | 62 | 195 | <0.001 | 283 | 24 | 259 | 0.023 |
| (grouped) | pT3a-b & pT4 | | 127 | 74 | 53 | | 177 | 29 | 148 | |
| Nodal status | pN0 | | 307 | 115 | 192 | <0.001 | 372 | 47 | 325 | 0.229 |
| | pN1 | | 16 | 12 | 4 | | 24 | 5 | 19 | |
| Surgical margin | negative | | 269 | 68 | 201 | <0.001 | 300 | 28 | 272 | 0.070 |
| status | positive | | 113 | 66 | 47 | | 154 | 25 | 129 | |
| Preoperative | <10 | ng/mL | 165 | 34 | 131 | <0.001 | 183 | 18 | 165 | 0.771 |
| PSA level | ≧10 | ng/mL | 219 | 103 | 116 | | 276 | 35 | 241 | |

Multivariate Cox regression analysis (Recurrence-free survival)

| | | Global | | | Stepwise backward selection | | |
|---|---|---|---|---|---|---|---|
| Variable | Characteristics | HR | 95% CI | P^{§} | HR | 95% CI | P^{§} |
| Age at diagnosis | <63 years | 0.983 | 0.674-1.434 | 0.929 | — | — | — |
| (grouped) | vs ≧63 years | | | | | | |
| Gleason score | 5-7 vs 8-10 | 1.626 | 1.059-2.496 | 0.026 | 1.643 | 1.077-2.505 | 0.021 |
| (grouped) | | | | | | | |
| Tumors stage | pT2a-c vs | 1.909 | 1.240-2.940 | 0.003 | 1.946 | 1.281-2.958 | 0.002 |
| (grouped) | pT3a-c & pT4 | | | | | | |
| Nodal status | pN0 vs pN1 | 1.254 | 0.627-2.510 | 0.522 | — | — | — |
| Surgical margin | negative vs | 1.915 | 1.271-2.884 | 0.002 | 1.933 | 1.287-2.902 | 0.001 |
| status | positive | | | | | | |
| Preoperative | <10 ng/mL | 2.027 | 1.270-3.235 | 0.003 | 2.009 | 1.259-3.206 | 0.003 |
| PSA level | vs ≧10 ng/mL | | | | | | |
| Cytoplasmic | negative vs | 0.504 | 0.342-0.745 | 0.001 | 0.485 | 0.333-0.709 | <0.001 |
| SLC18A2 IHC | score 1 + to 3+ | | | | | | |
| Nuclear | negative vs | | | | | | |
| SLC18A2 IHC | score 1 + to 3+ | 0.888 | 0.535-1.475 | 0.647 | — | — | — |

*Tumor recurrence was defined as PSA levels ≧0.1 ng/mL at least one month post-operatively. For Kaplan-Meier plots see FIG. 9
^{†}See FIG. 13 for Kaplan-Meier plots.
^{‡}Log-rank test, two-sided; bold face mark p-values <0.05.
^{§}P-values <0.05 are marked in bold.

Using a global multivariate Cox regression model, high Gleason score (p=0.026), high tumor stage (p=0.003), positive surgical margins (p=0.002), high preoperative PSA (p=0.003), and negative cytoplasmic SLC18A2 staining (p=0.001) were significantly associated with shorter RFS times (Table 1). After reverse selection, the same five variables remained in the model, strongly indicating that negative cytoplasmic SLC18A2 immunoreactivity is an independent adverse risk factor for prostate cancer recurrence. Our concomitant identification of four already established risk factors for recurrence supports the validity of this finding. The hazard ratio (HR) for biochemical recurrence in patients with positive cytoplasmic SLC18A2 staining was 0.485 (95% confidence interval 0.333-0.709).

When evaluated as a test for prediction of PSA recurrence within 5 years, cytoplasmic SLC18A2 IHC analysis (negative or positive) had a sensitivity of 0.48, specificity of 0.82, positive predictive value of 0.69, and negative predictive value of 0.67.

### Example 6

### The CpG Site Located at −969 Base Pairs of SLC18A2 is Hypermethylated in Prostate Cancer

The DNA methylation of the SLC18A2 promoter CpG island located at −969 base pairs of SLC18A2 was investigated in prostate cancer tissue samples using Infinium HumanMethylation27 BeadChip (Illumina). The probe “cg00498305”, having the sequence 5′-CGGCCCATTTCAGGGACACTGAGGCTCAAAGAGCCACGTGATTTATTCAA-3′, interrogates the CpG site at position −969 upstream of the ATG site (FIG. 14)

The CpG site located at −969 is hypermethylated in PC tissue samples (FIG. 15, T1-8, black bars) compared to adjacent non-malignant prostate tissue samples (FIG. 15, N1-10 black bars).

Mean methylation in non-malignant samples is 0.368 (Mean methylation value is determined as a beta-value=signal intensity from methylated probe/total signal intensity from unmethylated+methylated probe on Infinium 27K BeadChip)

Mean methylation in prostate cancer samples is 0.602 (beta-value)

Fold-increase of methylation in prostate cancer compared to non-malignant prostate cancer calculated without background adjustment is: 0.602/0.368=1.6

Fold-increase of methylation in prostate cancer compared to non-malignant prostate cancer calculated with assay background adjustment: (0.602−0.2)/(0.368−0.2)=2.6

Thus, fold-increase of methylation in cancer compared to non-malignant prostate ranges from 1.6 to 2.6 fold for the CpG site at pos.-969:

The DNA methylation of the SLC18A2 promoter CpG island located at −214 base pairs of SLC18A2 was investigated in prostate cancer tissue samples using Infinium HumanMethylation27 BeadChip (Illumina). The probe “cg00512279”, having the sequence 5′-AGCGTCCTGGGGGTTCTAGGTTGGGTCTCCAGATTGGGTCCCCGACGGCG-3′, interrogates a CpG site at position −214 upstream of the ATG site (FIG. 14). The CpG site located at −214 base pairs of SLC18A2 is unmethylated in all samples and hence not relevant as a biomarker (FIG. 15, grey bars).

The methylation of SLC18A2 at position −969 base pairs (determined by Illumina Infunium 27K BeadChip analysis; probe “cg00498305”) in clinical prostate cancer tissue samples was compared with SLC18A2 transcript levels (determined by Affymetrix Exon Array analysis). There is a clear inverse correlation (Pearson correlation coefficient=−0.66) between SLC18A2 transcript levels and methylation of SLC18A2 at position −969 in clinical prostate cancer tissue samples (see FIG. 16).

These results further support that hypermethylation of the SLC18A2 promoter region downregulates expression of SLC18A2.

### REFERENCES

1. Nelson W G, Yegnasubramanian S, Agoston A T, et al. Abnormal DNA methylation, epigenetics, and prostate cancer. Front Biosci 2007; 12: 4254-66.
2. Humphrey P A. Gleason grading and prognostic factors in carcinoma of the prostate. Mod Pathol 2004; 17: 292-306.
3. Yegnasubramanian S, Kowalski J, Gonzalgo M L, et al. Hypermethylation of CpG islands in primary and metastatic human prostate cancer. Cancer Res 2004; 64: 1975-86.
4. Dobosy J R, Roberts J L, Fu V X, Jarrard D F. The expanding role of epigenetics in the development, diagnosis and treatment of prostate cancer and benign prostatic hyperplasia. Urol 2007; 177: 822-31.
5. Jones P A, Baylin S B. The fundamental role of epigenetic events in cancer. Nat Rev Genet. 2002; 3: 415-28.
6. Weihe E, Eiden L E. Chemical neuroanatomy of the vesicular amine transporters. FASEB J 2000; 14: 2435-49.
7. Uccella S, Cerutti R, Vigetti D, et al. Histidine decarboxylase, DOPA decarboxylase, and vesicular monoamine transporter 2 expression in neuroendocrine tumors: immunohistochemical study and gene expression analysis. J Histochem Cytochem 2006; 54: 863-75.
8. Jakobsen A M, Andersson P, Saglik G, et al. Differential expression of vesicular monoamine transporter (VMAT) 1 and 2 in gastrointestinal endocrine tumours. J Pathol 2001; 195: 463-72.
9. Graff L, Castrop F, Bauer M, Hofler H, Gratzl M. Expression of vesicular monoamine transporters, synaptosomal-associated protein 25 and syntaxin1: a signature of human small cell lung carcinoma. Cancer Res 2001; 61: 2138-44.
10. Fon E A, Pothos E N, Sun B C, Killeen N, Sulzer D, Edwards R H. Vesicular transport regulates monoamine storage and release but is not essential for amphetamine action. Neuron 1997; 19: 1271-83.
11. Takahashi N, Miner L L, Sora I, et al. VMAT2 knockout mice: heterozygotes display reduced amphetamine-conditioned reward, enhanced amphetamine locomotion, and enhanced MPTP toxicity. Proc Natl Acad Sci USA 1997; 94: 9938-43.
12. Wang Y M, Gainetdinov R R, Fumagalli F, et al. Knockout of the vesicular monoamine transporter 2 gene results in neonatal death and supersensitivity to cocaine and amphetamine. Neuron 1997; 19: 1285-96.
13. Abdul M, Anezinis P E, Logothetis C J, Hoosein N M. Growth inhibition of human prostatic carcinoma cell lines by serotonin antagonists. Anticancer Res 1994; 14: 1215-20.
14. Dizeyi N, Bjartell A, Nilsson E, et al. Expression of serotonin receptors and role of serotonin in human prostate cancer tissue and cell lines. Prostate 2004; 59: 328-36.
15. Siddiqui E J, Shabbir M, Mikhailidis D P, Thompson C S, Mumtaz F H. The role of serotonin (5-hydroxytryptamine 1A and 1B) receptors in prostate cancer cell proliferation. J Urol 2006; 176: 1648-53.
16. Valencia S, Hernandez-Angeles A, Soria-Jasso L E, Arias-Montano J A. Histamine H(1) receptor activation inhibits the proliferation of human prostatic adenocarcinoma DU-145 cells. Prostate 2001; 48: 179-87.
17. Lang K, Drell T L, Lindecke A, et al. Induction of a metastatogenic tumor cell type by neurotransmitters and its pharmacological inhibition by established drugs. Int J Cancer 2004; 112: 231-8.
18. Palm D, Lang K, Niggemann B, et al. The norepinephrine-driven metastasis development of PC-3 human prostate cancer cells in BALB/c nude mice is inhibited by beta-blockers. Int J Cancer 2006; 118: 2744-9.
19. Sastry K S, Karpova Y, Prokopovich S, et al. Epinephrine protects cancer cells from apoptosis via activation of cAMP-dependent protein kinase and BAD phosphorylation. J Biol Chem 2007; 282: 14094-100.
20. Thorsen K, Sørensen K D, Brems-Eskildsen A S, et al. Alternative splicing in colon, bladder, and prostate cancer identified by exon-array analysis. Mol Cell Proteomics 2008; 7:1214-24.
21. Halvorsen O J, Oyan A M, Bo T H, et al. Gene expression profiles in prostate cancer: association with patient subgroups and tumour differentiation. Int J Oncol 2005; 26: 329-36.
22. Magee J A, Araki T, Patil S, et al. Expression profiling reveals hepsin overexpression in prostate cancer. Cancer Res 2001; 61: 5692-6.
23. Vanaja D K, Cheville J C, Iturria S J, Young C Y. Transcriptional silencing of zinc finger protein 185 identified by expression profiling is associated with prostate cancer progression. Cancer Res 2003; 63: 3877-82.
24. Varambally S, Yu J, Laxman B, et al. Integrative genomic and proteomic analysis of prostate cancer reveals signatures of metastatic progression. Cancer Cell 2005; 8: 393-406.
25. Yu Y P, Landsittel D, Jing L, et al. Gene expression alterations in prostate cancer predicting tumor aggression and preceding development of malignancy. J Clin Oncol 2004; 22: 2790-9.
26. Epstein J I, Allsbrook W C, Jr., Amin M B, Egevad L L. The 2005 International Society of Urological Pathology (ISUP) Consensus Conference on Gleason Grading of Prostatic Carcinoma. Am J Surg Pathol 2005; 29: 1228-42.
27. Kononen J, Bubendorf L, Kallioniemi A, et al. Tissue microarrays for high-throughput molecular profiling of tumor specimens. Nat Med 1998; 4: 844-7.
28. Heeboll S, Borre M, Ottosen P D, et al. SMARCC1 expression is upregulated in prostate cancer and positively correlated with tumour recurrence and dedifferentiation. Histol Histopathol 2008; 23: 1069-76.
29. Sørensen K D, Borre M, Ørntoft T F, Dyrskjøt L, Tørring N. Chromosomal deletion, promoter hypermethylation and downregulation of FYN in prostate cancer. Int J Cancer 2008; 122: 509-19.
30. Thiriot D S, Ruoho A E. Mutagenesis and derivatization of human vesicle monoamine transporter 2 (VMAT2) cysteines identifies transporter domains involved in tetrabenazine binding and substrate transport. J Biol Chem 2001; 276: 27304-15.
31. Tørring N, Borre M, Sørensen K D, Andersen C L, Wiuf C, Ørntoft T F. Genome-wide analysis of allelic imbalance in prostate cancer using the Affymetrix 50K SNP mapping array. Br J Cancer 2007; 96: 499-506.
32. Fernandez P L, Farre X, Nadal A, et al. Expression of cathepsins B and S in the progression of prostate carcinoma. Int J Cancer 2001; 95: 51-5.
33. Tang S, Bhatia B, Maldonado C J, et al. Evidence that arachidonate 15-lipoxygenase 2 is a negative cell cycle regulator in normal prostate epithelial cells. J Biol Chem 2002; 277: 16189-201.
34. Jassen A K, Brown J M, Panas H N, Miller G M, Xiao D, Madras B K. Variants of the primate vesicular monoamine transporter-2. Brain Res Mol Brain Res 2005; 139: 251-7.
35. Eiden L E, Schafer M K, Weihe E, Schutz B. The vesicular amine transporter family (SLC18): amine/proton antiporters required for vesicular accumulation and regulated exocytotic secretion of monoamines and acetylcholine. Pflugers Arch 2004; 447: 636-40.
36. Burke P A, Gregg J P, Bakhtiar B, et al. Characterization of MUC1 glycoprotein on prostate cancer for selection of targeting molecules. Int J Oncol 2006; 29: 49-55.
37. Cruz-Muros I, Afonso-Oramas D, Abreu P, Rodriguez M, Gonzalez M C, Gonzalez-Hernandez T. Deglycosylation and subcellular redistribution of VMAT2 in the mesostriatal system during normal aging. Neurobiol Aging 2007; 29:1702-11.
38. Nirenberg M J, Liu Y, Peter D, Edwards R H, Pickel V M. The vesicular monoamine transporter 2 is present in small synaptic vesicles and preferentially localizes to large dense core vesicles in rat solitary tract nuclei. Proc Natl Acad Sci USA 1995; 92: 8773-7.
39. Yao J, Hersh L B. The vesicular monoamine transporter 2 contains trafficking signals in both its N-glycosylation and C-terminal domains. J Neurochem 2007; 100: 1387-96.
40. Erickson J D, Schafer M K, Bonner T I, Eiden L E, Weihe E. Distinct pharmacological properties and distribution in neurons and endocrine cells of two isoforms of the human vesicular monoamine transporter. Proc Natl Acad Sci USA 1996; 93: 5166-71.
41. Nadelhaft I. Cholinergic axons in the rat prostate and neurons in the pelvic ganglion. Brain Res 2003; 989: 52-7.
42. Abdul M, Logothetis C J, Hoosein N M. Growth-inhibitory effects of serotonin uptake inhibitors on human prostate carcinoma cell lines. J Urol 1995; 154: 247-50.
43. Cox M E, Deeble P D, Lakhani S, Parsons S J. Acquisition of neuroendocrine characteristics by prostate tumor cells is reversible: implications for prostate cancer progression. Cancer Res 1999; 59: 3821-30.
44. Ramos-Jimenez J, Soria-Jasso L E, Lopez-Colombo A, Reyes-Esparza J A, Camacho J, Arias-Montano J A. Histamine augments beta2-adrenoceptor-induced cyclic AMP accumulation in human prostate cancer cells DU-145 independently of known histamine receptors. Biochem Pharmacol 2007; 73: 814-23.
45. Ayala G E, Dai H, Ittmann M, et al. Growth and survival mechanisms associated with perineural invasion in prostate cancer. Cancer Res 2004; 64: 6082-90.
Wojdacz T K, Dobrovic A, Hansen L L. Methylation-sensitive high-resolution melting. 2008., Nat. Protoc. 2008; 3(12):1903-8.
Tost, J. & Gut, I. G. DNA methylation analysis by pyrosequencing, Nature Protocols 2, -2265-2275 (2007)

1. A method for assisting in diagnosing and/or for diagnosing prostate cancer in an individual comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of presence or absence of prostate cancer.

2. The method according to claim 1, wherein an increased methylation status is indicative of the presence of prostate cancer.

3. The method according to claim 1, wherein a decreased transcriptional and/or translational expression level in the sample is indicative of the presence of prostate cancer.

4. A method for assisting in prognosing and/or for prognosing the disease progression of prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of the progression of prostate cancer.

5. The method according to claim 4, wherein an increased methylation status is indicative of the disease progression of prostate cancer.

6. The method according to claim 4, wherein a decreased transcriptional and/or translational expression level in the sample is indicative of the disease progression of prostate cancer.

7. A method for assisting in predicting and/or for predicting the outcome of prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of the outcome of prostate cancer.

8. The method according to claim 7, wherein an increased methylation status is indicative of the disease progression of prostate cancer.

9. The method according to claim 7, wherein a decreased transcriptional and/or translational expression level in the sample is indicative of the disease progression of prostate cancer.

10. A method for assisting in predicting and/or for predicting the recurrence risk of prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of the recurrence risk of prostate cancer.

11. The method according to claim 10, wherein an increased methylation status is indicative of the recurrence risk of prostate cancer.

12. The method according to claim 10, wherein a decreased transcriptional and/or translational expression level in the sample is indicative of the recurrence risk of prostate cancer.

13. (canceled)

14. A method for assisting in monitoring and/or for monitoring the effect of treatment on prostate cancer progression in an individual having contracted prostate cancer, comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of progression of said prostate cancer.

15. The method according to claim 14, wherein an increased methylation status is indicative of a progression of said prostate cancer.

16. The method according to claim 14, wherein a decreased transcriptional and/or translational expression level in the sample is indicative a progression of said prostate cancer.

17. A method for assisting in monitoring and/or for monitoring the progression of prostate cancer from a silent/indolent to an aggressive prostate cancer in an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of the progression of prostate cancer from a silent/indolent to an aggressive prostate cancer.

18. (canceled)

19. (canceled)

20. The method according to claim 17, wherein an increased methylation status is indicative of a progression of said prostate cancer.

21. The method according to claim 17, wherein a decreased transcriptional and/or translational expression level in the sample is indicative a progression of said prostate cancer.

22. A method for assisting in determining and/or determining the treatment regime of an individual having contracted prostate cancer comprising the steps of
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of the treatment regime to be offered to the individual having contracted prostate cancer.

23. The method according to claim 22, wherein an increased methylation status is indicative of the presence and/or the progression of said prostate cancer.

24. The method according to claim 22, wherein a decreased transcriptional and/or translational expression level in the sample is indicative of the presence and/or the progression of said prostate cancer.

25. The method according to claim 1, wherein the method further comprises a step of comparing the methylation status of said SLC18A2 gene determined in the sample to the methylation status of a control sample, and wherein the methylation status of said sample can be determined as increased or decreased.

26. The method according to claim 1, wherein the methylation status of said SLC18A2 gene in a sample being >1% is indicative of an increased methylation level, and wherein said sample is a tissue sample.

27. The method according to claim 1, wherein the methylation status of said SLC18A2 gene in a sample being >0% is indicative of an increased methylation level, and wherein said sample is a urine sample.

28. The method of claim 25, wherein the methylation status of said SLC18A2 gene in a sample is increased in comparison with said control sample.

29. The method of claim 28, wherein said control sample is a fully unmethylated DNA fragment.

30. The method according to claim 1, wherein the method further comprises a step of comparing the transcriptional and/or translational expression level of the SLC18A2 gene determined in the sample to the expression level of a control sample, and wherein the expression level in the sample can be determined as increased or decreased.

31. (canceled)

32. The method according to claim 1, wherein the transcriptional expression level of the SLC18A2 gene in a sample is decreased by 10% in comparison with the transcriptional level of a control sample.

33. (canceled)

34. The method according to claim 1, wherein the translational expression level of the SLC18A2 gene is decreased by 10% in comparison with the translational expression level of a control sample.

35. The method according to claim 1, wherein the sample is selected from tissue sample, blood, plasma, serum, semen, or urine.

36. The method according to claim 1, wherein the sample is a biopsy of the prostate gland or resected prostate tissue following radical prostatectomy.

37. The method according to claim 1, wherein the method further comprise a step of measuring the level of prostate specific antigen (PSA) in s individual.

38. (canceled)

39. A method for treatment of an individual comprising the steps of
i) selecting an individual having contracted prostate cancer, wherein the methylation status of the SLC18A2 gene is increased, and/or the transcriptional and/or translational expression level of the SLC18A2 gene is decreased, determined according to the method of claim 1, and
ii) treating the individual with methylation modulating agent in a therapeutically effective amount.

40. The method according to claim 39, wherein said methylation modulating agent is capable of
i) reducing the methylation status of a SLC18A2 gene (SEQ ID NO: 1), or nucleotide sequence having at least 90% sequence identity with SEQ ID NO:1, or part thereof, and/or
ii) increasing the transcriptional and/or translational expression level of the SLC18A2 gene.

41. The method according to claim 39, wherein said methylation modulating agent is selected from the group consisting of DNA methylation inhibitors and histone deacetylases.

42. A method for reducing tumorigenicity of a cell, said method comprising the steps of
i) providing
a) at least one SLC18A2 gene (SEQ ID NO:1), or nucleotide sequence having at least 90% sequence identity with SEQ ID NO:1, or part thereof,
b) at least one SLC18A2 gene transcript (SEQ ID NO: 2), or nucleotide sequence having at least 90% sequence identity with SEQ ID NO:2, or part thereof, and/or
c) at least one translational product of the SLC18A2 gene (SEQ ID NO: 3), or nucleotide sequence having at least 90% sequence identity with SEQ ID NO:3, or part thereof; and
ii) introducing at least one of a), b), and c) of step i) into the tumor cell.

43. A kit comprising at least one detection member for a SLC18A2 gene, transcriptional and/or translational product or part thereof for use in the methods of the present invention.

44. The kit according to claim 43, wherein said at least one detection member is selected from the group consisting of an antibody directed against an epitope of the SLC18A2 protein or part thereof, and oligonucleotides, primers and/or probes.

45. The kit according to claim 43, further comprising means for providing the level of methylation or transcription or translation of said SLC18A2 gene (SEQ ID NO:1) or nucleotide sequence having at least 90% sequence identity with SEQ ID NO:1 or part thereof and/or means for providing information as to whether the level is above or below a cut off value.

46. The method according to claim 1, wherein a translational expression level of a SLC18A2 gene is detected by detecting an epitope of the SLC18A2 translational product as shown in SEQ ID NO:3, or a variant or part thereof.

47. A method for determining the status of an individual having prostate cancer or suspected of having prostate cancer, the method comprising the steps of:
i) determining the methylation status of a SLC18A2 gene (SEQ ID NO:1), or nucleotide sequence having at least 90% sequence identity with SEQ ID NO:1, or part thereof in a sample from said individual, and/or
ii) determining the transcriptional and/or translational expression level of said SLC18A2 gene, or part thereof in said sample;
wherein the methylation status of i) and/or the transcriptional and/or the translational expression level of ii) is indicative of the status of said individual.
